SEQUENCE LISTING (111 sequences)

SEQ ID NO: 1 - DNA, 7389 bp, Human enterovirus B
ttaaaacagcctgtgggttgttcccacccacaggcccattgggcgctagcactctggtatcacggtacctttgtgcgcctgttttacatcccctccccaaattgtaatttagaagtttcacacaccgatcattagcaagcgtggcacaccagccatgttttgatcaagcacttctgttaccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaaatgggggctcaagtatcaacacaaaagactggagcacatgagaccgggctgaatgcaagcgggaattccatcatccattacacaaacataaactactataaagatgctgcatccaactcagccaacagacaggatttcactcaagacccaggtaaatttactgagccagtgaaggacattatgatcaaatcgatgccagcattgaactccccctctgccgaagagtgtggttatagtgacagggttcgatcaataaccctcgggaactccactatcacaacacaggagtgtgctaatgttgtggttgggtatggtgtgtggccagaatatttgaaggacaatgaagccacaggcgaggaccaaccaacacaacccgacgtggccacatgtcggttctacactttagagtccgtgcagtggatgaagaattcagcaggttggtggtggaagttaccagatgcactttcacaaatggggttgtttggacaaaatatgcagtaccactacttagggagaacaggctacaccatccacgtgcagtgcaacgcatctaagtttcatcagggctgtctactagtggtgtgtgtgccggaagcggaaatgggatgctcaaatctaaacaacaccccaaagtttgctgaactttccggaggagataatgctagaatgttcactgacaccgaagtggggacatccaacgataagaaagtacaaacagcagtgtggaatgcaggtatgggtgttggtgttggcaacctgactatattcccacaccagtggatcaacctgaggacaaacaatagtgctaccattgtaatgccttacatcaacagcgttcccatggacaacatgtatagacacaacaacttgacactgatgataatcccattcgtcccgctcaactatagtgagggatcatcaccttacgtaccaatcacagtcacaattgcgccaatgtgcgcagaatacaatggccttagactggctagtagccagggtttgcccgtcatgacgacaccgggtagcacgcaatttctgacttctgatgatttccaatcgccatcagcaatgccacaattcgacgtcacaccagagatgcagatcccgggtcgtgtcaacaatctgatggaaatagctgaggtggactctgtggtcccagtgaacaacactgacaacaacgtgaatggtcttaaagcctaccaaatccccgtgcaatccaatagcgacaatagaaggcaagtgttcgggtttcccttacaacctggggccaacaatgttctcaacaggactttgttaggtgagattttgaactactacacccactggagtggtagtataaagctcacgttcatgttttgtggatcggcgatggccactgggaagtttttgttagcatactcaccaccaggcgcaggggttccaaaaaacagaagagacgcaatgttgggtacacatgtcatctgggacgtgggtttgcaatcaagttgtgtactttgtgtaccctggatcagccagacacactataggtatgtggtggaggatgagtatacagcggcagggtatgttacatgctggtaccaaacaaatatcattgttccagcggacgttcaaagcacatgcgacatcttgtgcttcgtatcggcatgcaatgatttctctgtgcgtatgttaaaggataccccttttataagacaagacaatttttaccagggcccagtggaagaatcggtggaacgcgccatggtgcgcgtggctgatacggtcagctcaaagcctacgaattcagaatccatccccgccctcactgctgcggagactggtcatacctctcaagtcgtacccagtgacaccatgcaaactagacacgtgaagaattaccactcaaggtcggaatcttccattgaaaatttcttgtgccgatctgcttgtgtgtattacgccacctacaataataattctgagaagggctacgcagagtgggttatcaacaccaggcaggttgcgcagttgctgaggaggaaactagagttcacttacttaaggttcgatctggaattgacatttgtgataacaagtgcccaggagcccagtaccgctaccagtgtagacgcacctgtacaaacccaacagataatgtatgtgcctccaggcgggccggtacctaccaaggttacagactacgcctggcaaacatccacaaaccccagcgtcttttggacagaggggaatgccccaccgaggatgtctattcccttcatcagtattggaaatgcttatagctgcttttatgatggatggacacagttctctaggaatggggtttacggcattaataccctgaacaacatgggtacgctgtacatgcggcacgtaaatgaggcaggccaaggcccgatcaaaagtacagttagaatatattttaaacccaaacatgtgaaggcgtgggtgcccagaccgccgaggctatgtcaatatgagaagcaaaagaatgttaatttcaacccaacaggagtcaccacaacgcgctcaaacattaccacaaccggcgcttttggacaacagtcaggggctgtctacgtaggcaactacagagtggtgaatagacacctcgccacccgcgaggactggcagcggtgcgtgtgggaagattacaacagagatcttcttgtgagtacaaccacggcacatggatgcgatatcatagccaggtgtcagtgcacaaccggggtttatttctgtgcgtcaaggaataagcactatccagtttcctttgaggggccgggtcttgtggaggttcaagagagtgagtactatccaaagaggtaccaatcccacgtgcttttagcggcaggcttttccgaaccaggggattgtggtggaatcctaaggtgcgaacatggtgtcgtgggcattgtaactatgggaggggagggtgttgttggctttgccgacgtacgcgacctgctgtggttagaagatgatgcaatggagcagggggtcaaagattacgtagagcaacttggaaacgcctttgggtcaggattcactaaccaagtctgtgaacaggttaaccttttgaaagagtccctagtgggccaagactccatcttagaaaaatctctgaaagccctagtaaaaattgtgtcagccttggtgatagtcgtgaggaaccacgacgatttaataacagtgactgcaacacttgccctcattggctgcacctcctcaccatggagatggcttaagcaaaaggtgtcccagtactatggcattccaatggctgagcgccagaacagtggttggctcaagaagttcactgaaatgaccaacgcctgcaagggaatggagtggatagctattaaaatccagaaatttatagaatggctcaaagttaaaatcctaccagaagttaaagaaaaacatgagttcttaaacagacttaaacaacttccgttactggagagtcagattgccaccattgagcaaagtgcaccttctcagagtgatcaagagcaattgttttctaatgttcagtactttgcacattactgcaggaaatacgcacccctgtatgcggccgaagcaaaaagggtgttctcccttgagaagaaaatgagtaactacatacagttcaagtccaaatgccgtattgaacctgtatgcttgctcttgcacgggagtcctggtgcagggaagtcagttgccactaacctgattgggcgctcactcgcggagaagcttaatagctctgtgtactctctaccaccagacccagaccattttgacggttacaaacaacaggccgttgtgatcatggatgatttatgccagaacccagatggtaaggatgtttctttgttttgtcaaatggtctccagtgtggactttgtaccgccaatggcagcgctagaagagaaaggcattttgttcacctctccctttgtcctggcttcaaccaacgccggctctattaatgctccaacagtttcagacagtagggcactggctaggaggtttcactttgatatgaacatcgaggtcatttccatgtacagccagaatgggaagataaacatgcctatgtcggtgaagacgtgtgatgaagagtgttgcccagtcaactttaagaaatgctgccccttagtgtgtggaaaggctatccagtttatagatagaaggacacaagtcagatactcccttgacatgctggtaactgagatgttcagagaatataaccatagacatagtgttggggccacccttgaggcgctatttcagggcccgccaatatatagagagattaagatcagtattgcgccagagacgccaccaccacctgccattgcagatctactcaaatcagtagacagtgaggctgtgagggagtactgcaaagaaaaaggatggctggttccagagatcaactccaccctgcagattgaaaagcacgtcagtagggcctttatctgtttgcaagcattgactacttttgtctcagtagcaggaatcatctacataatatataaactcttcgcaggctttcaaggagcatacacgggaatgcccaatcagaagcccaaggtacccactctccgtcaagcaaaagtacaaggacctgtatttgagtttgccgtagccatgatgaagaggaattcgagcacagtgaaaactgaatacggtgagttcactatgctgggtatctatgataggtgggctgttttgccacgtcatgccaaacctgggccaaccatcttaatgaatgatcaagaaattggtgtgctagacgccaaggagttagtggacaaggacggtacaaacttggaactgacactacttaagctgaaccgaaatgaaaagttcagagatattaggggcttcttagccaaggaagaggtggaggttaatgaggctgtgctagcaattaacaccagcaagttccctaacatgtatatcccagtgggacaagttacagagtatggcttcctgaatctgggtggtacacccaccaagagaatgcttatgtacaacttccccacgagggcaggtcagtgtggcggagtgctcatgtccactggcaaagttttggggatccatgttggcgggaatggtcatcaaggcttctcagcggcacttcttaaacactacttcaatgatgaacaaggagaaatcgagttcattgaaagctcaaaggaagcagggttcccagtcatcaacacgcctagcaaaactaaattggagccaagtgtcttccaccaggtttttgaaggcaataaagagccagcagtcctcaggaatggcgatccacgcctcagagccaactttgaggaggccatattttccaaatacatcggaaatgttaacacacacgtggatgaatacatgctggaagctgttgaccattatgctggtcaattggcaaccttagacatcagcaccgagccaatgagattggaagatgctgtatatggtactgaaggccttgaggctcttgatttaacaacgagtgcgggctacccgtatgtcgctctgggcattaaaaagagggacatcctctcaaagaagaccagagacctgaccaagctgaaggagtgcatggacaagtatggcctaaacctaccaatggtaacctatgtgaaagatgaactcagatctgcagagaaggtggcgaaagggaaatctaggctaattgaggcatccagtttgaatgactccgtggcaatgagacaaacatttggcaacctatacaaaacttttcacctaaacccagggattgtaactggcagtgccgtcgggtgtgatccagatctcttttggagcaagataccagtgatgttggatggccatctcgtagcttttgattattctggatatgacgctagcttgagtcccgtatggtttgcctgtctaaagctactgcttgagaaacttggttactcgcacaaggagaccaattacattgactacctgtgcaactcccatcacctgtacagggataaacattactttgtgcggggtggcatgccatcaggatgctctggcacaagcatcttcaactcaatgataaataacatcataatcaggacactaatgttgaaggtgtacaaagggatcgacttggatcaattcagaatgattgcttatggtgatgacgtgatcgcatcatatccgtggcccatagatgcgtccctgcttgctgaagctggtagggattgtgggttgatcatgacaccagcagacaaaggggattgcttcaatgaagtcacctgggctaatgtcacatttctaaagaggtattttagagcagatgaacagtacccctttttagtgcaccccgtcatgcctatgaaagacatacatgaatcaatcagatggaccaaggacccaaagaacacccaagatcatgtgcgctcgttgtgcttattggcctggcacaacggggagcacgaatatgaggagttcatccgcaaaattagaagcgtcccagttggagcttgcttgactctacctgcgttttcaactctacgcaggaagtggttggactctttttaaattagagacaatttgaactaattttaattggcttaaccctaccgcactaaccgaactagataacggtgcagtgggggtaaattctccgcattcggtgcg

SEQ ID NO: 2 - DNA, 19 bp, Artificial Sequence
  Description of Artificial Sequence Synthetic primer
ttcctccggcccctgaatg

SEQ ID NO: 3 - DNA, 21 bp, Artificial Sequence
  Description of Artificial Sequence Synthetic primer
tccggcccctgaatgcggcta

SEQ ID NO: 4 - DNA, 17 bp, Artificial Sequence
  Description of Artificial Sequence Synthetic primer
tggctgcgttggcggcc

SEQ ID NO: 5 - DNA, 23 bp, Artificial Sequence
  Description of Artificial Sequence Synthetic primer
tctactttgggtgtccgtgtttc

SEQ ID NO: 6 - DNA, 22 bp, Artificial Sequence
  Description of Artificial Sequence Synthetic primer
tcagcctgtgggttgtacccac

SEQ ID NO: 7 - DNA, 20 bp, Artificial Sequence
  Description of Artificial Sequence Synthetic primer
ttctagcctgcgtggctgcc

SEQ ID NO: 8 - DNA, 20 bp, Artificial Sequence
  Description of Artificial Sequence Synthetic primer
tcctccggcgccgtgaatgc

SEQ ID NO: 9 - DNA, 19 bp, Artificial Sequence
  Description of Artificial Sequence Synthetic primer
tcccacccatggggcccac

SEQ ID NO: 10 - DNA, 18 bp, Artificial Sequence
  Description of Artificial Sequence Synthetic primer
tcctccggcccctgaatg

SEQ ID NO: 11 - DNA, 21 bp, Artificial Sequence
  Description of Artificial Sequence Synthetic primer
tggtacctttgtacgcctgtt

SEQ ID NO: 12 - DNA, 32 bp, Artificial Sequence
  Description of Artificial Sequence Synthetic primer
tatggtgatgatgtaattgcttcctaccccca

SEQ ID NO: 13 - DNA, 26 bp, Artificial Sequence
  Description of Artificial Sequence Synthetic primer
tactcaacattgtaccgccgttggct

SEQ ID NO: 14 - DNA, 21 bp, Artificial Sequence
  Description of Artificial Sequence Synthetic primer
taagggcggcatgccatctgg

SEQ ID NO: 15 - DNA, 22 bp, Artificial Sequence
  Description of Artificial Sequence Synthetic primer
tgcatgttggtgggaacggttc

SEQ ID NO: 16 - DNA, 27 bp, Artificial Sequence
  Description of Artificial Sequence Synthetic primer
tcaaatcactgagacaaatgatggagt

SEQ ID NO: 17 - DNA, 26 bp, Artificial Sequence
  Description of Artificial Sequence Synthetic primer
tgaaccccaccagaaatctggtcgtg

SEQ ID NO: 18 - DNA, 26 bp, Artificial Sequence
  Description of Artificial Sequence Synthetic primer
tgaacaactacatgcagttcaagagc

SEQ ID NO: 19 - DNA, 23 bp, Artificial Sequence
  Description of Artificial Sequence Synthetic primer
taaccctactgcgctaaccgaac

SEQ ID NO: 20 - DNA, 27 bp, Artificial Sequence
  Description of Artificial Sequence Synthetic primer
tgagcaattacatacagttcaagtcca

SEQ ID NO: 21 - DNA, 30 bp, Artificial Sequence
  Description of Artificial Sequence Synthetic primer
taggatgatcgcttatggtgatgatgtgat

SEQ ID NO: 22 - DNA, 21 bp, Artificial Sequence
  Description of Artificial Sequence Synthetic primer
tggcatctatgacaggtgggc

SEQ ID NO: 23 - DNA, 24 bp, Artificial Sequence
  Description of Artificial Sequence Synthetic primer
tagtaaccctacctcagccgaatt

SEQ ID NO: 24 - DNA, 27 bp, Artificial Sequence
  Description of Artificial Sequence Synthetic primer
tctcctagcccaatcaggaaaagacta

SEQ ID NO: 25 - DNA, 37 bp, Artificial Sequence
  Description of Artificial Sequence Synthetic primer
tgccaatgaaagaaatttatgaatcaattagatggac

SEQ ID NO: 26 - DNA, 34 bp, Artificial Sequence
  Description of Artificial Sequence Synthetic primer
taacattcctgaaaagattcttcagagcagatga

SEQ ID NO: 27 - DNA, 25 bp, Artificial Sequence
  Description of Artificial Sequence Synthetic primer
tgtgtcacttaatgtggcacaatgg

SEQ ID NO: 28 - DNA, 29 bp, Artificial Sequence
  Description of Artificial Sequence Synthetic primer
tattttgatggttatgatcagcagagtgt

SEQ ID NO: 29 - DNA, 21 bp, Artificial Sequence
  Description of Artificial Sequence Synthetic primer
tgattacacatggcagagtgc

SEQ ID NO: 30 - DNA, 24 bp, Artificial Sequence
  Description of Artificial Sequence Synthetic primer
tggatgtgatggttctccatggag

SEQ ID NO: 31 - DNA, 24 bp, Artificial Sequence
  Description of Artificial Sequence Synthetic primer
tgaaacacgggcaccgaaagtagt

SEQ ID NO: 32 - DNA, 27 bp, Artificial Sequence
  Description of Artificial Sequence Synthetic primer
tgaaacacggacacccaaagtagtcgg

SEQ ID NO: 33 - DNA, 21 bp, Artificial Sequence
  Description of Artificial Sequence Synthetic primer
tagccgcattcaggggccgga

SEQ ID NO: 34 - DNA, 22 bp, Artificial Sequence
  Description of Artificial Sequence Synthetic primer
tcattgtcaccataagcagcca

SEQ ID NO: 35 - DNA, 23 bp, Artificial Sequence
  Description of Artificial Sequence Synthetic primer
taaaacaggcgcacaagggtacc

SEQ ID NO: 36 - DNA, 23 bp, Artificial Sequence
  Description of Artificial Sequence Synthetic primer
tagcacacgcgggtcttcacacc

SEQ ID NO: 37 - DNA, 26 bp, Artificial Sequence
  Description of Artificial Sequence Synthetic primer
tgaaacacggagtcccgaaagtagtc

SEQ ID NO: 38 - DNA, 23 bp, Artificial Sequence
  Description of Artificial Sequence Synthetic primer
tgggaaaacaggcgtacaaaggt

SEQ ID NO: 39 - DNA, 23 bp, Artificial Sequence
  Description of Artificial Sequence Synthetic primer
tgaaacacggacacccaaagtag

SEQ ID NO: 40 - DNA, 22 bp, Artificial Sequence
  Description of Artificial Sequence Synthetic primer
tccggggaaacagaagtgcttg

SEQ ID NO: 41 - DNA, 29 bp, Artificial Sequence
  Description of Artificial Sequence Synthetic primer
tagtcttttcctgattgggctaggagact

SEQ ID NO: 42 - DNA, 29 bp, Artificial Sequence
  Description of Artificial Sequence Synthetic primer
taacccaatccaattcgactgaggtaggg

SEQ ID NO: 43 - DNA, 30 bp, Artificial Sequence
  Description of Artificial Sequence Synthetic primer
taggtggtctaaatctatgcccttgtaggt

SEQ ID NO: 44 - DNA, 26 bp, Artificial Sequence
  Description of Artificial Sequence Synthetic primer
tgagtgaagtatgatcgctttagggc

SEQ ID NO: 45 - DNA, 25 bp, Artificial Sequence
  Description of Artificial Sequence Synthetic primer
tgttggggtacttgctagtgttcac

SEQ ID NO: 46 - DNA, 32 bp, Artificial Sequence
  Description of Artificial Sequence Synthetic primer
tattctggttataacaaattcacccccaccag

SEQ ID NO: 47 - DNA, 25 bp, Artificial Sequence
  Description of Artificial Sequence Synthetic primer
tcagacatacaggttcaatacggtg

SEQ ID NO: 48 - DNA, 24 bp, Artificial Sequence
  Description of Artificial Sequence Synthetic primer
tcaccgaatgcggagaatttaccc

SEQ ID NO: 49 - DNA, 24 bp, Artificial Sequence
  Description of Artificial Sequence Synthetic primer
taagcatacaggttcaatacggca

SEQ ID NO: 50 - DNA, 23 bp, Artificial Sequence
  Description of Artificial Sequence Synthetic primer
tctcctttgtctgctggtgtcat

SEQ ID NO: 51 - DNA, 21 bp, Artificial Sequence
  Description of Artificial Sequence Synthetic primer
tccaggtttggcatggcgtgg

SEQ ID NO: 52 - DNA, 29 bp, Artificial Sequence
  Description of Artificial Sequence Synthetic primer
tccgaattaaaggaaaatttacccctaca

SEQ ID NO: 53 - DNA, 30 bp, Artificial Sequence
  Description of Artificial Sequence Synthetic primer
tctgctctgaagaatcttttcaggaatgtt

SEQ ID NO: 54 - DNA, 24 bp, Artificial Sequence
  Description of Artificial Sequence Synthetic primer
tccgttgtgccaagccaataggca

SEQ ID NO: 55 - DNA, 33 bp, Artificial Sequence
  Description of Artificial Sequence Synthetic primer
tgattcatgaatttctttcattggcattactgg

SEQ ID NO: 56 - DNA, 24 bp, Artificial Sequence
  Description of Artificial Sequence Synthetic primer
tgggatatacagtgcgcgaccagc

SEQ ID NO: 57 - DNA, 24 bp, Artificial Sequence
  Description of Artificial Sequence Synthetic primer
tggattttgcatgatgtcatccat

SEQ ID NO: 58 - DNA, 29 bp, Artificial Sequence
  Description of Artificial Sequence Synthetic primer
tcatcatgtgagtaaccatcatagaaaca

SEQ ID NO: 59 - DNA, 28 bp, Artificial Sequence
  Description of Artificial Sequence Synthetic primer
tgaaccagccatcatttgcttgcctttc

SEQ ID NO: 60 - DNA, 21 bp, Artificial Sequence
  Description of Artificial Sequence Synthetic primer
tggatgtccagaaggtacccc

SEQ ID NO: 61 - DNA, 23 bp, Artificial Sequence
  Description of Artificial Sequence Synthetic primer
tgccaaaagccacgtgtataaga

SEQ ID NO: 62 - DNA, 20 bp, Artificial Sequence
  Description of Artificial Sequence Synthetic primer
tgggttaccgtgtggcagct

SEQ ID NO: 63 - DNA, 22 bp, Artificial Sequence
  Description of Artificial Sequence Synthetic primer
tcaagcctggcaaagacaggat

SEQ ID NO: 64 - DNA, 25 bp, Artificial Sequence
  Description of Artificial Sequence Synthetic primer
tgagagtgtggagtacagatggagg

SEQ ID NO: 65 - DNA, 24 bp, Artificial Sequence
  Description of Artificial Sequence Synthetic primer
tggcgaagacggtgaagcagatgg

SEQ ID NO: 66 - DNA, 21 bp, Artificial Sequence
  Description of Artificial Sequence Synthetic primer
tgtgtgccctatttgcacagc

SEQ ID NO: 67 - DNA, 20 bp, Artificial Sequence
  Description of Artificial Sequence Synthetic primer
tactgcgatagtgccacccc

SEQ ID NO: 68 - DNA, 23 bp, Artificial Sequence
  Description of Artificial Sequence Synthetic primer
tggacgatgatgtcttctggcct

SEQ ID NO: 69 - DNA, 18 bp, Artificial Sequence
  Description of Artificial Sequence Synthetic primer
tgtgcgcgggtaccatgc

SEQ ID NO: 70 - DNA, 24 bp, Artificial Sequence
  Description of Artificial Sequence Synthetic primer
tatggctcacctggaaaggaaagg

SEQ ID NO: 71 - DNA, 23 bp, Artificial Sequence
  Description of Artificial Sequence Synthetic primer
tcactgcatttggggcagacagc

SEQ ID NO: 72 - DNA, 22 bp, Artificial Sequence
  Description of Artificial Sequence Synthetic primer
tgaaaaccacgaccacgtggtt

SEQ ID NO: 73 - DNA, 19 bp, Artificial Sequence
  Description of Artificial Sequence Synthetic primer
tggggtaccttctgggcat

SEQ ID NO: 74 - DNA, 20 bp, Artificial Sequence
  Description of Artificial Sequence Synthetic primer
tggggcaggtgagatgggca

SEQ ID NO: 75 - DNA, 24 bp, Artificial Sequence
  Description of Artificial Sequence Synthetic primer
tcatgacaggtcgatacagagggc

SEQ ID NO: 76 - DNA, 22 bp, Artificial Sequence
  Description of Artificial Sequence Synthetic primer
tgtccgcagtatgacgaccgtg

SEQ ID NO: 77 - DNA, 25 bp, Artificial Sequence
  Description of Artificial Sequence Synthetic primer
taaggtccctgctcttgctcatcca

SEQ ID NO: 78 - DNA, 24 bp, Artificial Sequence
  Description of Artificial Sequence Synthetic primer
taggccagaagacatcatcgtcca

SEQ ID NO: 79 - DNA, 19 bp, Artificial Sequence
  Description of Artificial Sequence Synthetic primer
tgggccggaaaatgctgac

SEQ ID NO: 80 - DNA, 27 bp, Artificial Sequence
  Description of Artificial Sequence Synthetic primer
tgactgggagttactcttgtcagatga

SEQ ID NO: 81 - DNA, 19 bp, Artificial Sequence
  Description of Artificial Sequence Synthetic primer
tagcaccccaccttgtggc

SEQ ID NO: 82 - DNA, 21 bp, Artificial Sequence
  Description of Artificial Sequence Synthetic primer
tcgcctatcaacagctgggta

SEQ ID NO: 83 - DNA, 25 bp, Artificial Sequence
  Description of Artificial Sequence Synthetic primer
tccaatcaacatccgggtcagttcc

SEQ ID NO: 84 - DNA, 32 bp, Artificial Sequence
  Description of Artificial Sequence Synthetic primer
tcaaccacagtttctacagaacagaatgttcc

SEQ ID NO: 85 - DNA, 34 bp, Artificial Sequence
  Description of Artificial Sequence Synthetic primer
tgtgatgacagttgaaattaggaaacaaaatatg

SEQ ID NO: 86 - DNA, 27 bp, Artificial Sequence
  Description of Artificial Sequence Synthetic primer
tgaaagtcagagaatgatgaaagtgga

SEQ ID NO: 87 - DNA, 28 bp, Artificial Sequence
  Description of Artificial Sequence Synthetic primer
tgttcaatgaatgtagtctccaaaacgc

SEQ ID NO: 88 - DNA, 27 bp, Artificial Sequence
  Description of Artificial Sequence Synthetic primer
tgctcagtggtttgcttttatgcatgg

SEQ ID NO: 89 - DNA, 33 bp, Artificial Sequence
  Description of Artificial Sequence Synthetic primer
tcagtcctgcttggagaaagagatgatagaata

SEQ ID NO: 90 - DNA, 27 bp, Artificial Sequence
  Description of Artificial Sequence Synthetic primer
tgtgtggacttttgagatggatgctgg

SEQ ID NO: 91 - DNA, 27 bp, Artificial Sequence
  Description of Artificial Sequence Synthetic primer
tggatgtgtgagatgggtcatgaatgc

SEQ ID NO: 92 - DNA, 30 bp, Artificial Sequence
  Description of Artificial Sequence Synthetic primer
tgcagtggctgagtttttccagtcttttcc

SEQ ID NO: 93 - DNA, 24 bp, Artificial Sequence
  Description of Artificial Sequence Synthetic primer
tgttgggagtggtcttgaccacat

SEQ ID NO: 94 - DNA, 21 bp, Artificial Sequence
  Description of Artificial Sequence Synthetic primer
tccagggatgtgtggtggggc

SEQ ID NO: 95 - DNA, 30 bp, Artificial Sequence
  Description of Artificial Sequence Synthetic primer
tgctaggtttacggatttggagctgcatgg

SEQ ID NO: 96 - DNA, 30 bp, Artificial Sequence
  Description of Artificial Sequence Synthetic primer
ttgaaataccattcttatgcaagatttggc

SEQ ID NO: 97 - DNA, 30 bp, Artificial Sequence
  Description of Artificial Sequence Synthetic primer
tatgttttgtttttggagaggagaccttgt

SEQ ID NO: 98 - DNA, 27 bp, Artificial Sequence
  Description of Artificial Sequence Synthetic primer
tggtgcttggactcctgaaacatccat

SEQ ID NO: 99 - DNA, 31 bp, Artificial Sequence
  Description of Artificial Sequence Synthetic primer
tggaaaagactggaaaaactcagctactgca

SEQ ID NO: 100 - DNA, 29 bp, Artificial Sequence
  Description of Artificial Sequence Synthetic primer
tgcgttttggagactacattcattgaaca

SEQ ID NO: 101 - DNA, 33 bp, Artificial Sequence
  Description of Artificial Sequence Synthetic primer
tgcaggaaaattaatcatgtttttatcaatgtg

SEQ ID NO: 102 - DNA, 28 bp, Artificial Sequence
  Description of Artificial Sequence Synthetic primer
tctttctccaaacaggactgaacaaaat

SEQ ID NO: 103 - DNA, 32 bp, Artificial Sequence
  Description of Artificial Sequence Synthetic primer
tggtcataaaacctcattctccaccaatcata

SEQ ID NO: 104 - DNA, 32 bp, Artificial Sequence
  Description of Artificial Sequence Synthetic primer
tcatccttcatttctgtccatttttcatcatt

SEQ ID NO: 105 - DNA, 36 bp, Artificial Sequence
  Description of Artificial Sequence Synthetic primer
tcctctattgaaataaaactccatcatttcataatc

SEQ ID NO: 106 - DNA, 21 bp, Artificial Sequence
  Description of Artificial Sequence Synthetic primer
tgcagctccaacagcaaccca

SEQ ID NO: 107 - DNA, 29 bp, Artificial Sequence
  Description of Artificial Sequence Synthetic primer
tccacagaagtaaaataagaagcaccagt

SEQ ID NO: 108 - DNA, 33 bp, Artificial Sequence
  Description of Artificial Sequence Synthetic primer
tccagcaacatgaatgcccaaaatggcattctg

SEQ ID NO: 109 - DNA, 33 bp, Artificial Sequence
  Description of Artificial Sequence Synthetic primer
taaagacattttggctcttgcatcttcataatt

SEQ ID NO: 110 - DNA, 29 bp, Artificial Sequence
  Description of Artificial Sequence Synthetic primer
tgctggaatggtgttggatttatctgaac

SEQ ID NO: 111 - DNA, 30 bp, Artificial Sequence
  Description of Artificial Sequence Synthetic primer
tgaatgatatttggtgggaaagacttgaaa

(19) United States
(12) Patent Application Publication
(10) Pub. No.: US 2011/0097704 A1
(43) Pub. Date: Apr. 28, 2011

(54) COMPOSITIONS FOR USE IN IDENTIFICATION OF PICORNAVIRUSES

(75) Inventors: Rangarajan Sampath, San Diego, CA (US); Lawrence B. Blyn, Mission Viejo, CA (US)

(73) Assignee: IBIS Biosciences, Inc., Carlbad, CA (US)

(21) Appl. No.: 12/864,977

(22) PCT Filed: Jan. 27, 2009
(86) PCT No.: PCT/US09/32156

§ 371 Date: Nov. 22, 2010

Related U.S. Application Data
(60) Provisional application No. 61/024,425, filed on Jan. 29, 2008.
(60) Provisional application No. 61/057,625, filed on May 30, 2008.

(51) Int. Cl. C12Q 1/70; C12Q 1/68; C12M 1/34

(52) U.S. Cl. 435/5; 435/6; 435/287.1; 435/287.2

(57) ABSTRACT

The present invention provides oligonucleotide primers, compositions, and kits containing the same for rapid identification of viruses which are members of the Picornaviridae family by amplification of a segment of viral nucleic acid followed by molecular mass analysis.

### PRIORITY

This application claims priority to provisional patent application Ser. No. 61/024,425, filed Jan. 29, 2008, and 61/057,625, filed May 30, 2008, each of which is herein incorporated by reference in its entirety.

### STATEMENT OF GOVERNMENT SUPPORT

This invention was made with United States government support under Army SBIR USAMRAA contract No. W81XWH-06-C-0050 awarded by the United States Army, and under NIH/NIAID contract No. HHSN2662004-00100C/N01-AIK-40100 awarded by the National Institutes of Health. The United States government has certain rights in the invention.

### FIELD OF THE INVENTION

The present invention relates generally to the field of genetic identification and quantification of human Picornavirus and provides methods, compositions and kits useful for this purpose when combined, for example, with molecular mass or base composition analysis.

### SEQUENCE LISTING

The present application is being filed along with a Sequence Listing in electronic format. The Sequence Listing is provided as a file entitled DIBIS0099USL2SEQ.txt, created Mar. 13, 2008, which is 26.8 Kb in size. The information in the electronic format of the sequence listing is incorporated herein by reference in its entirety.

### BACKGROUND OF THE INVENTION

Picornaviruses represent a very large virus family of small ribonucleic acid (RNA)-containing viruses responsible for many serious human and animal diseases (Rueckert, R. R. Virology, 2nd ed. (Fields, B. N. et al., eds.) Raven Press, Ltd., New York, p. 508-548 (1982)). Examples of Picornaviruses include rhinoviruses, enteroviruses (e.g. poliovirus, coxsackievirus, echovirus), cardioviruses (e.g. encephalomyocarditis virus, meningovirus), and hepatoviruses (e.g. hepatitis A virus), among others. These viruses are associated with a wide range of human diseases including summer flu, diarrhea, meningitis, hepatitis, pneumonia, myocarditis, pericarditis, and diabetes (Melnick, J. L. Virology, 2nd ed. (Fields, B. N. et al., eds.) Raven Press, Ltd., New York p 549-605).

Enteroviruses (genus Enterovirus, family Picornaviridae) constitute a broad range of pathogens etiologically responsible for a wide range of diseases in both humans and in other animals. Enteroviruses are small RNA viruses that contain positive, single stranded RNA as the genome. Five groups are found within the enteroviruses: coxsackievirus A, coxsackievirus B, echovirus, poliovirus, and the numbered enteroviruses.

Rhinovirus is also a genus of the Picornaviridae family of viruses. Rhinoviruses are small RNA viruses that contain positive, single-stranded RNA genomes, which are typically between 7.2 and 8.5 kb in length. To further illustrate, human rhinoviruses (HRVs) are one of the major causes of upper respiratory tract infections collectively known as the common cold. In addition, human rhinoviruses include a large number of serotypes (i.e. at least 100 serotypes), which tends to make detection and identification of the virus challenging.

Cardiovirus is another genus within the Picornaviridae family. The cardiovirus genus presently includes two species, namely, Encephalomyocarditis virus and Theilovirus. Encephalomyocarditis virus is represented by a single serotype of the same name while the Theiloviruses are comprised of Theiler's murine encephalomyelitis virus (TMEV), Vilyuisk human encephalomyelitis virus (VHEV) and a Theiler's-like virus isolated from rats (TLV).

To further illustrate, Hepatovirus is another genus within the Picornaviridae family. The genus Hepatovirus consists of two species, Hepatitis A virus and (the as yet unnamed) “Avian encephalomyelitis-like viruses”. Hepatitis A is an acute infectious disease of the liver caused by Hepatovirus hepatitis A virus. Sufferers, especially children, may exhibit no symptoms, making detection of the disease difficult.

Thus, there is a need in the art for assays and other aspects related to the rapid detection and characterization of members of the Picornaviridae family.

### SUMMARY OF THE INVENTION

Provided herein are, inter alia, compositions, kits, and methods of identifying members of the Picornaviridae family. In some embodiments, the genus of the members is identified. In some embodiments the species of the members is identified. In some embodiments, the sub-species of the members is identified. In some embodiments, the strain of the members is identified. In some embodiments, the genotype of the members is identified. Also provided are oligonucleotide primers, compositions and kits containing oligonucleotide primers that upon amplification, produce amplicons whose molecular masses provide the means to identify, for example, Enteroviruses, Cardioviruses, Hepatoviruses and Rhinoviruses at the sub-species level. In certain embodiments, related systems of use in the detection and identification of members of the Picornaviridae family are also provided.

In some embodiments, the invention provides primers, and compositions comprising pairs of primers; kits containing the same; and methods for their use in the identification of members of the Picornaviridae family, such as Enteroviruses, Rhinoviruses, Cardioviruses or Hepatoviruses. The primers are typically configured to produce viral bioagent-identifying nucleic acid amplicons i.e. amplification products. The amplicons are typically generated from regions of nucleic acid encoding genes essential to virus replication. Compositions comprising pairs of primers and the kits containing the same are generally configured to provide species and sub-species characterization of, for example, Enteroviruses, Cardioviruses, Hepatoviruses and Rhinoviruses.

In another aspect, the invention provides a composition comprising at least one purified oligonucleotide primer pair that comprises forward and reverse primers, wherein the primer pair comprises nucleic acid sequences that are substantially complementary to nucleic acid sequences of two or more different bioagents belonging to the Picornaviridae family, wherein the primer pair is configured to produce amplicons comprising different base compositions that correspond to (i.e., match, identify, or otherwise correlate with) said two or more different bioagents. In some embodiments, the primer pair is configured to hybridize with conserved regions of two or more different bioagents and flank variable regions of the two or more different bioagents. In further embodiments, the forward and reverse primers are about 15 to 35 nucleobases in length, and the forward primer comprises at least 70%, at least 80%, at least 90%, at least 95%, or at least 100% sequence identity with a sequence of SEQ ID NOS: 2-30, 60-71 and 84-97, and the reverse primer comprises at least 70% sequence identity with a sequence of SEQ ID NOS: 31-59, 72-83, and 98-111. In still further embodiments, the primer pair is one or more of: SEQ ID NOS: 2:31, 3:32, 4:33, 5:34, 6:35, 7:36, 8:37, 9:38, 10:39, 11:40, 12:41, 13:42, 4:43, 15:44, 16:45, 17:46, 18:47, 19:48, 20:49, 21:50, 22:51, 23:52, 24:53, 25:54, 26:55, 27:56, 28:57, 29:58, 30:59, 60:72, 61:73, 62:74, 63:75, 64:76, 65:77, 66:78, 67:79, 68:80, 69:81, 70:82, 71:83, 84:98, 85:99, 86:100, 87:101, 88:102, 89:103, 90:104, 91:105, 92:106, 93:107, 94:108, 95:109, 96:110, and 97:111. In some embodiments, the forward and reverse primers are about 15 to 35 nucleobases in length, and the forward primer comprises at least 70%, at least 80%, at least 90%, at least 95%, or at least 100% sequence identity with the sequence of SEQ ID NO: 2, and the reverse primer comprises at least 70%, at least 80%, at least 90%, at least 95%, or at least 100% sequence identity with the sequence of SEQ ID NO: 31; the forward primer comprises at least 70%, at least 80%, at least 90%, at least 95%, or at least 100% sequence identity with the sequence of SEQ ID NO: 3, and the reverse primer comprises at least 70%, at least 80%, at least 90%, at least 95%, or at least 100% sequence identity with the sequence of SEQ ID NO: 32; the forward primer comprises at least 70%, at least 80%, at least 90%, at least 95%, or at least 100% sequence identity with the sequence of SEQ ID NO: 10, and the reverse primer comprises at least 70%, at least 80%, at least 90%, at least 95%, or at least 100% sequence identity with the sequence of SEQ ID NO: 39; the forward primer comprises at least 70%, at least 80%, at least 90%, at least 95%, or at least 100% sequence identity with the sequence of SEQ ID NO: 4, and the reverse primer comprises at least 70%, at least 80%, at least 90%, at least 95%, or at least 100% sequence identity with the sequence of SEQ ID NO: 33; the forward primer comprises at least 70%, at least 80%, at least 90%, at least 95%, or at least 100% sequence identity with the sequence of SEQ ID NO: 5, and the reverse primer comprises at least 70%, at least 80%, at least 90%, at least 95%, or at least 100% sequence identity with the sequence of SEQ ID NO: 34; the forward primer comprises at least 70%, at least 80%, at least 90%, at least 95%, or at least 100% sequence identity with the sequence of SEQ ID NO: 7, and the reverse primer comprises at least 70%, at least 80%, at least 90%, at least 95%, or at least 100% sequence identity with the sequence of SEQ ID NO: 36; the forward primer comprises at least 70%, at least 80%, at least 90%, at least 95%, or at least 100% sequence identity with the sequence of SEQ ID NO: 61, and the reverse primer comprises at least 70%, at least 80%, at least 90%, at least 95%, or at least 100% sequence identity with the sequence of SEQ ID NO: 73; and/or, the forward primer comprises at least 70%, at least 80%, at least 90%, at least 95%, or at least 100% sequence identity with the sequence of SEQ ID NO: 64, and the reverse primer comprises at least 70%, at least 80%, at least 90%, at least 95%, or at least 100% sequence identity with the sequence of SEQ ID NO: 76.

In some embodiments, the different base compositions identify two or more different bioagents at the genus, species, or sub-species levels. In other embodiments, the two or more amplicons are 45 to 200 nucleobases in length. In still other embodiments, the different bioagents are selected from the group consisting of: Enterovirus A species, Enterovirus B species, Enterovirus C species, Enterovirus D species, Poliovirus species, Rhinovirus genus, Rhinovirus A species, Rhinovirus B species, Coxsackievirus genus, Coxsackievirus A species, Coxsackievirus B species, Coxsackievirus C species, Porcine enterovirus species, Bovine enterovirus species, Hepatovirus genus, Cardiovirus genus, or combinations thereof. In further embodiments, the primer pair is configured to hybridize with one or more nucleic acid sequences selected from the group consisting of, e.g., WTPV1, WTHRV14, WTCVB3, PCV305, PV5′HRV14, GENOME5UTR, GENOMEUTR, POLIO5UTR, POLIO3D, POLIO3C, COXA3D, COXA2C, COXB3D, COXB2C, COXB3C, COXC3D, HRVA3D, HRVA2C, HRVBVP1, HRVB2B, HAV, CARDIOVIRUS5UTR, CARDIOVIRUS1C, CARDIOVIRUS3D, CARDIOVIRUS3AB, THEILOVIRUS5UTR, THEILOVIRUS5UTR-1A, THEILOVIRUS2A-2B, THEILOVIRUS2C, and THEILOVIRUS3D nucleic acids. As noted further below, certain primer pair designs produce levels of identification sought within the Picornaviridae family, whereas other designs failed.

In some embodiments, a non-templated T residue on the 5′-end of said forward and/or reverse primer is removed. In still other embodiments, the forward and/or reverse primer further comprises a non-templated T residue on the 5′-end. In additional embodiments, the forward and/or reverse primer comprises at least one molecular mass modifying tag. In some embodiments, the forward and/or reverse primer comprises at least one modified nucleobase. In further embodiments, the modified nucleobase is 5-propynyluracil or 5-propynylcytosine. In other embodiments, the modified nucleobase is a mass modified nucleobase. In still other embodiments, the mass modified nucleobase is 5-Iodo-C. In additional embodiments, the modified nucleobase is a universal nucleobase. In some embodiments, the universal nucleobase is inosine. In certain embodiments, kits comprise the compositions described herein.

In another aspect, the invention provides a kit comprising at least one purified oligonucleotide primer pair that comprises forward and reverse primers that are about 20 to 35 nucleobases in length, and wherein the forward primer comprises at least 70%, at least 80%, at least 90%, at least 95%, or at least 100% sequence identity with a sequence selected from the group consisting of SEQ ID NOS: 2-30, 60-71, and 84-97, and the reverse primer comprises at least 70% sequence identity with a sequence selected from the group consisting of SEQ ID NOS: 31-59, 72-83 and 98-111.

In another aspect, the invention provides a method of determining a presence of a picornavirus in at least one sample. The method includes (a) amplifying one or more segments of at least one nucleic acid from said sample using at least one purified oligonucleotide primer pair that comprises forward and reverse primers that are about 20 to 35 nucleobases in length, and wherein said forward primer comprises at least 70%, at least 80%, at least 90%, at least 95%, or at least 100% sequence identity with a sequence selected from the group consisting of SEQ ID NOs: 2-30, 60-71, and 84-97, and said reverse primer comprises at least 70% sequence identity with a sequence selected from the group consisting of SEQ ID NOs: 31-59, 72-83, and 98-111 to produce at least one amplification product. In addition, the method also includes (b) detecting said amplification product, thereby determining said presence of said picornavirus in said sample. In some embodiments, (a) comprises amplifying said one or more segments of said at least one nucleic acid from at least two samples obtained from different geographical locations to produce at least two amplification products, and (b) comprises detecting said amplification products, thereby tracking an epidemic spread of said picornavirus. Optionally, (b) comprises determining an amount of said picornavirus in said sample (e.g., determining a viral load or the like). Typically, (b) comprises detecting a molecular mass of said amplification product. In some embodiments, (b) comprises determining a base composition of said amplification product in which said base composition identifies the number of A residues, C residues, T residues, G residues, U residues, analogs thereof and/or mass tag residues thereof in said amplification product, whereby said base composition indicates the presence of picornavirus in said sample or identifies said picornavirus in said sample. In certain embodiments, the method includes comparing said base composition of said amplification product to calculated or measured base compositions of amplification products of one or more known picornaviruses present in a database with the proviso that sequencing of said amplification product is not used to indicate the presence of or to identify said picornavirus in which a match between said determined base composition and said calculated or measured base composition in said database indicates the presence of or identifies said picornavirus.

In another aspect, the invention provides a method of identifying one or more picornavirus bioagents in a sample. The method includes (a) amplifying two or more segments of a nucleic acid from said one or more picornavirus bioagents in said sample with two or more oligonucleotide primer pairs to obtain two or more amplification products; (b) determining two or more molecular masses and/or base compositions of said two or more amplification products; and (c) comparing said two or more molecular masses and/or said base compositions of said two or more amplification products with known molecular masses and/or known base compositions of amplification products of known picornavirus bioagents produced with said two or more primer pairs to identify said one or more picornavirus bioagents in said sample. In some embodiments, the method includes identifying said one or more picornavirus bioagents in said sample using three, four, five, six, seven, eight or more primer pairs. Optionally, said two or more segments of said nucleic acid are amplified from a single gene, or said two or more segments of said nucleic acid are amplified from different genes. In some embodiments, said one or more picornavirus bioagents in said sample cannot be identified using a single primer pair of said two or more primer pairs. Typically, the method includes obtaining said two or more molecular masses of said two or more amplification products via mass spectrometry. In certain embodiments, said one or more picornavirus bioagents in said sample cannot be identified using a single primer pair of said two or more primer pairs.

In some embodiments, said picornavirus bioagents are selected from the group consisting of: an Enterovirus genus, a Rhinovirus genus, a Hepatovirus genus, a Cardiovirus genus, an Aphthovirus genus, a Parechovirus genus, an Erbovirus genus, a Kobuvirus genus, a Teschovirus genus, a species thereof, a sub-species thereof, and combinations thereof Optionally, said two or more primer pairs comprise two or more purified oligonucleotide primer pairs that each comprise forward and reverse primers that are about 20 to 35 nucleobases in length, and wherein said forward primers comprise at least 70%, at least 80%, at least 90%, at least 95%, or at least 100% sequence identity with a sequence selected from the group consisting of SEQ ID NOS: 2-30, 60-71, and 84-97, and said reverse primers comprise at least 70% sequence identity with a sequence selected from the group consisting of SEQ ID NOS: 31-59, 72-83, and 98-111 to obtain an amplification product. In some embodiments, said primer pairs are selected from the group of primer pair sequences consisting of: SEQ ID NOS: 2:31, 3:32, 4:33, 5:34, 6:35, 7:36, 8:37, 9:38, 10:39, 11:40, 12:41, 13:42, 4:43, 15:44, 16:45, 17:46, 18:47, 19:48, 20:49, 21:50, 22:51, 23:52, 24:53, 25:54, 26:55, 27:56, 28:57, 29:58, 30:59, 60:72, 61:73, 62:74, 63:75, 64:76, 65:77, 66:78, 67:79, 68:80, 69:81, 70:82, 71:83, 84:98, 85:99, 86:100, 87:101, 88:102, 89:103, 90:104, 91:105, 92:106, 93:107, 94:108, 95:109, 96:110, and 97:111.

Typically, said determining said two or more molecular masses and/or base compositions is conducted without sequencing said two or more amplification products. In some embodiments, said one or more picornavirus bioagents in a sample are identified by comparing three or more molecular masses and/or base compositions of three or more amplification products with a database of known molecular masses and/or known base compositions of amplification products of known picornavirus bioagents produced with said three or more primer pairs. In certain embodiments, the method includes calculating said two or more base compositions from said two or more molecular masses of said two or more amplification products.

In some embodiments, members of said primer pairs hybridize to conserved regions of said nucleic acid that flank a variable region. Typically, said variable region varies between at least two of said picornavirus bioagents. In some embodiments, said variable region uniquely varies between at least five of said picornavirus bioagents.

In certain embodiments, said two or more amplification products obtained in (a) comprise major classification and subgroup identifying amplification products. In some embodiments, the method includes comparing said molecular masses and/or said base compositions of said two or more amplification products to calculated or measured molecular masses or base compositions of amplification products of known picornavirus bioagents in a database comprising genus specific amplification products, species specific amplification products, strain specific amplification products or nucleotide polymorphism specific amplification products produced with said two or more oligonucleotide primer pairs in which one or more matches between said two or more amplification products and one or more entries in said database identifies said one or more picornavirus bioagents, classifies a major classification of said one or more picornavirus bioagents, and/or differentiates between subgroups of known and unknown picornavirus bioagents in said sample. In some of these embodiments, said major classification of said one or more picornavirus bioagents comprises a genus or species classification of said one or more picornavirus bioagents. In some of these embodiments, said subgroups of known and unknown picornavirus bioagents comprise family, strain and nucleotide variations of said one or more picornavirus bioagents.

In another aspect, the invention provides a system that includes (a) a mass spectrometer configured to detect one or more molecular masses of amplicons produced using at least one purified oligonucleotide primer pair that comprises forward and reverse primers in which said primer pair comprises nucleic acid sequences that are substantially complementary to nucleic acid sequences of two or more different picornavirus bioagents. The system also includes (b) a controller operably connected to said mass spectrometer, said controller configured to correlate said molecular masses of said amplicons with one or more picornavirus bioagent identities (e.g., at genus, species, and/or sub-species levels). In some embodiments, said forward and reverse primers are about 15 to 35 nucleobases in length, and wherein the forward primer comprises at least 70%, at least 80%, at least 90%, at least 95%, or at least 100% sequence identity with a sequence selected from the group consisting of SEQ ID NOS: 2-30, 60-71 and 84-97, and the reverse primer comprises at least 70% sequence identity with a sequence selected from the group consisting of SEQ ID NOS: 31-59, 72-83, and 98-111. In certain embodiments, said primer pair is selected from the group of primer pair sequences consisting of: SEQ ID NOS: 2:31, 3:32, 4:33, 5:34, 6:35, 7:36, 8:37, 9:38, 10:39, 11:40, 12:41, 13:42, 4:43, 15:44, 16:45, 17:46, 18:47, 19:48, 20:49, 21:50, 22:51, 23:52, 24:53, 25:54, 26:55, 27:56, 28:57, 29:58, 30:59, 60:72, 61:73, 62:74, 63:75, 64:76, 65:77, 66:78, 67:79, 68:80, 69:81, 70:82, 71:83, 84:98, 85:99, 86:100, 87:101, 88:102, 89:103, 90:104, 91:105, 92:106, 93:107, 94:108, 95:109, 96:110, and 97:111. Typically, said controller is configured to determine (e.g., calculate, etc.) base compositions of said amplicons from said molecular masses of said amplicons, which base compositions correspond to (i.e., elucidate or otherwise correlate with) said one or more picornavirus bioagent identities. In some embodiments, said controller comprises or is operably connected to a database of known molecular masses and/or known base compositions of amplicons of known picornavirus bioagents produced with the primer pair.

In certain aspects, methods for identification of Picornaviruses, e.g., Enteroviruses, Rhinoviruses, Cardioviruses and Hepatoviruses are provided. Nucleic acid from the members of the Picornaviridae family is amplified using the primers described herein to obtain an amplicon. The molecular mass of the amplicon is measured using mass spectrometry. In some embodiments, a base composition of the amplicon is calculated from the molecular mass. As used herein, the term “base composition” refers to the number of each residue comprising an amplicon, without consideration for the linear arrangement of these residues in the strand(s) of the amplicon, wherein the base composition identifies the number of A residues, C residues, T residues, G residues, U residues, analogs thereof and/or mass tag residues thereof in said amplification product. The molecular mass or base composition is typically compared with a plurality of molecular masses or base compositions in a database of known Picornavirus identifying amplicons, wherein a match between the molecular mass or base composition and a member of the plurality of molecular masses or base compositions identifies the Picornavirus.

In some embodiments, methods of detecting the presence or absence of a Picornavirus in a sample are provided. Nucleic acid from the sample is amplified using the composition described above to obtain an amplicon. The molecular mass of this amplicon is determined by mass spectrometry. A base composition of the amplicon is determined from the molecular mass without sequencing the amplicon. The molecular mass or base composition of the amplicon is compared with known molecular masses or base compositions in a database of one or more known Picornavirus identifying amplicons, wherein a match between the molecular mass or base composition of the amplicon and the molecular mass or base composition of one or more known Picornavirus identifying amplicons indicates the presence of the Picornavirus in the sample.

In certain embodiments, methods for determination of the quantity of an unknown Picornavirus in a sample are provided. The sample is contacted with the composition described herein and a known quantity of a calibration polynucleotide. Nucleic acid from the unknown Picornavirus in the sample is concurrently amplified with the composition described above and nucleic acid from the calibration polynucleotide in the sample is concurrently amplified with the composition described above to obtain a first amplicon comprising a Picornavirus identifying amplicon and a second amplicon comprising a calibration amplicon. The molecular mass and abundance for the Picornavirus identifying amplicon and the calibration amplicon is determined by mass spectrometry. The Picornavirus identifying amplicon is distinguished from the calibration amplicon based on molecular mass, wherein comparison of Picornavirus identifying amplicon abundance and calibration amplicon abundance indicates the quantity of Picornavirus in the sample. The base composition of the Picornavirus identifying amplicon is determined.

In some embodiments, a method of identifying one or more Picornavirus bioagents in a sample is provided, comprising the steps of (a) amplifying two or more segments of a nucleic acid from said one or more of Picornavirus bioagents in the sample with two or more primer pairs to obtain two or more amplification products, wherein each of the primer pairs hybridizes to conserved regions of the nucleic acid that flank a variable region; (b) determining two or more molecular masses of the two or more amplification products; and (c) comparing the two or more molecular masses with a database containing known molecular masses of known Picornavirus bioagents produced with the two or more primer pairs to identify one or more Picornavirus bioagents in the sample. In some embodiments, the two or more primer pairs comprise two or more purified oligonucleotide primer pairs wherein the forward and reverse members of the two or more primer pairs are 20 to 35 nucleobases in length, and wherein the forward members comprises at least 70%, at least 80%, at least 90%, at least 95%, or at least 100% sequence identity with a sequence selected from the group consisting of SEQ ID NOS: 2-30, 60-71, and 84-97, and the reverse members comprises at least 70% sequence identity with a sequence selected from the group consisting of SEQ ID NOS: 31-59, 72-83, and 98-111 to obtain an amplification product. In other embodiments, the determining of two or more molecular masses of the two or more amplification products is conducted without sequencing. In further embodiments, the variable region varies between at least two or said Picornavirus bioagents. In still further embodiments, the variable region uniquely varies between at least five of said Picornavirus bioagents. In certain embodiments, the molecular masses of the two or more amplification products are obtained via mass spectrometry. In some embodiments, the one or more Picornavirus bioagents in the sample cannot be identified using a single primer pair of the two or more primer pairs. In additional embodiments, the one or more Picornavirus bioagents in a sample are identified by comparing three or more molecular masses to a database of bioagents produced with three or more primer pairs. In other embodiments, the two or more segments of a nucleic acid are amplified from a single gene. In still other embodiments, the two or more segments of a nucleic acid are amplified from different genes.

In some embodiments, a method of identifying one or more Picornavirus bioagents in a sample is provided, comprising (a) providing two or more oligonucleotide primer pairs wherein a forward member of the pair of primers hybridizes to a first conserved sequence of nucleic acid from the one or more picornavirus bioagents and a reverse member of the pair of primers hybridizes to a second conserved sequence of nucleic acid from the one or more picornavirus bioagents wherein the first and second conserved sequences flank a variable nucleic acid sequence that varies among different picornavirus bioagents; (b) providing nucleic acid from said sample; (c) amplifying two or more segments of the nucleic acid from the one or more picornavirus bioagents in the sample with the two or more oligoncleotide primer pairs to obtain two or more major classification and subgroup identifying amplification products; (d) determining molecular masses by mass spectrometry or base compositions by mass spectrometry of the two or more amplification products; and (e) comparing the molecular masses or the base compositions of the two or more amplification products to calculated or measured molecular masses or base compositions of amplification products of known Picornavirus bioagents in a database comprising genus specific amplification products, species specific amplification products, strain specific amplification products or nucleotide polymorphism specific amplification products produced with the two or more oligonucleotide primer pairs, wherein a match between the two or more amplification products and one or more entries in the database identifies the one or more picornavirus bioagents, and wherein a first match classifies a major classification of the one or more picornavirus bioagents, and a second match differentiates between subgroups of known and unknown picornavirus bioagents in the sample. In some embodiments, the major classification of the one or more Picornavirus bioagents comprises genus or species classification of the one or more Picornavirus bioagents. In other embodiments, the subgroups of known and unknown Picornavirus bioagents comprise family, strain and nucleotide variations of the one or more Picornavirus bioagents. In still other embodiments, the family of the one or more Picornavirus bioagents comprises the Picornaviridae family. In further embodiments, at least one of the two or more amplification products comprise nucleic acid sequences of the 5′ UTR of human Picornavirus. In still further embodiments, the amplification product comprises nucleic acid sequences of one or more of WTPV1, WTHRV14, WTCVB3, PCV305, PV5′HRV14, GENOME5UTR, GENOMEUTR, POLIO5UTR, POLIO3D, POLIO3C, COXA3D, COXA2C, COXB3D, COXB2C, COXB3C, COXC3D, HRVA3D, HRVA2C, HRVBVP1, HRVB2B, HAV, CARDIOVIRUS5UTR, CARDIOVIRUS1C, CARDIOVIRUS3D, CARDIOVIRUS3AB, THEILOVIRUS5UTR, THEILOVIRUS5UTR-1A, THEILOVIRUS2A-2B, THEILOVIRUS2C, and THEILOVIRUS3D. In some embodiments, the forward primer member comprises at least 70%, at least 80%, at least 90%, at least 95%, or at least 100% sequence identity with a sequence selected from the group consisting of SEQ ID NOs: 2-30, 60-71, and 84-97, and the reverse primer member comprises at least 70% sequence identity with a sequence selected from the group consisting of SEQ ID NOs: 31-59, 72-83, and 98-111. In additional embodiments, either or both of the members of the pair of primers comprises at least one modified nucleobase. In further embodiments, the modified nucleobase is a mass modified nucleobase or is a universal nucleobase. In still further embodiments, the universal nucleobase is inosine. In other embodiments, the mass modified nucleobase is 5-Iodo-C. In some embodiments, a non-templated T residue is added to the 5′-end on either or both of the primer pair members. In other embodiments, either or both of the forward and said reverse primer pair members further comprises a non-templated T residue on the 5′-end. In certain embodiments, the determining of the base compositions of the two or more amplification products is conducted without sequencing. In some embodiments, the variable sequence uniquely varies between at least five of said Picornavirus bioagents. In other embodiments, the base compositions of the two or more amplification products are calculated from molecular masses of the two or more amplification products. In still other embodiments, the one or more Picornavirus bioagents in the sample cannot be identified using a single primer pair of the two or more primer pairs. In further embodiments, the one or more Picornavirus bioagents in a sample are identified by comparing three or more base compositions to a database of Picornavirus bioagents produced with three or more primer pairs. In other embodiments, the two or more segments of the nucleic acid are amplified from a single gene. In still other embodiments, the two or more segments of the nucleic acid are amplified from different genes.

In some embodiments, a composition comprising a combination of at least three purified oligonucleotide primer pairs is provided, wherein the primer pairs hybridize to two or more genes selected from the group of WTPV1, WTHRV14, WTCVB3, PCV305, PV5′HRV14, GENOME5UTR, GENOMEUTR, POLIO5UTR, POLIO3D, POLIO3C, COXA3D, COXA2C, COXB3D, COXB2C, COXB3C, COXC3D, HRVA3D, HRVA2C, HRVBVP1, HRVB2B, HAV, CARDIOVIRUS5UTR, CARDIOVIRUS1C, CARDIOVIRUS3D, CARDIOVIRUS3AB, THEILOVIRUS5UTR, THEILOVIRUS5UTR-1A, THEILOVIRUS2A-2B, THEILOVIRUS2C, and THEILOVIRUS3D genes, wherein the primer pairs hybridize with conserved regions of the genes and flank variable regions of the genes to generate two or more amplicons from the two or more genes, wherein the two or more amplicons are configured to generate two or more molecular mass measurements using mass spectrometry, and wherein the two or more amplicons are configured to generate two or more base compositions from the molecular mass measurements that correspond to two or more unknown Picornavirus bioagents. In some embodiments, the primer pairs individually bind to one or more genes from the group of WTPV1, WTHRV14, WTCVB3, PCV305, PV5′HRV14, GENOME5UTR, GENOMEUTR, POLIO5UTR, POLIO3D, POLIO3C, COXA3D, COXA2C, COXB3D, COXB2C, COXB3C, COXC3D, HRVA3D, HRVA2C, HRVBVP1, HRVB2B, HAV, CARDIOVIRUS5UTR, CARDIOVIRUS1C, CARDIOVIRUS3D, CARDIOVIRUS3AB, THEILOVIRUS5UTR, THEILOVIRUS5UTR-1A, THEILOVIRUS2A-2B, THEILOVIRUS2C, and THEILOVIRUS3D genes.

In some embodiments, a method of tracking the epidemic spread of Picornavirus is provided, comprising (a) providing a one or more samples containing the Picornavirus from a plurality of locations; (b) providing Picornavirus RNA from the one or more samples; (c) providing DNA obtained from the RNA; (d) amplifying the DNA with a purified oligonucleotide primer pair wherein the forward and reverse members of said primer pair are 20 to 35 nucleobases in length, and wherein the forward primer comprises at least 70%, at least 80%, at least 90%, at least 95%, or at least 100% sequence identity with a sequence selected from the group consisting of SEQ ID NOS: 2-30, 60-71 and 84-97, and the reverse primer comprises at least 70% sequence identity with a sequence selected from the group consisting of SEQ ID NOS: 31-59, 72-83, and 98-111 to produce an amplification product; and (e)

identifying the Picornavirus in a subset of the one or more samples, wherein the amplification product identifies the Picornavirus and wherein the corresponding locations of the members of the subset indicate the epidemic spread of the Picornavirus to the corresponding locations. In some embodiments the method further comprises contacting the DNA with at least one primer pair comprising a forward member and a reverse member comprising oligonucleotide primers which hybridize to flanking sequences of the DNA, wherein the flanking sequences flank a variable DNA sequence corresponding to a variable RNA sequence of said picornavirus. In other embodiments, the method further comprises determining the base composition of the amplification product by mass spectrometry, wherein the base composition identifies the number of A residues, C residues, T residues, G residues, U residues, analogs thereof and mass tag residues thereof in the amplification product. I n further embodiments, the method further comprises comparing the base composition of the amplification product to calculated or measured base compositions of amplification products of one or more known Picornaviruses present in a database with the proviso that sequencing of the amplification product is not used to identify the Picornavirus, wherein a match between the determined base composition and the calculated or measured base composition in the database identifies the Picornavirus in the two or more samples. In certain embodiments the mass spectrometry comprises ESI-TOF mass spectrometry. In other embodiments, the one or more samples comprise at least one additional picornavirus selected from the group of Enterovirus A species, Enterovirus B species, Enterovirus C species, Enterovirus D species, Poliovirus genus, Rhinovirus genus, Rhinovirus A species, Rhinovirus B species, Coxsackievirus genus, Coxsackievirus A species, Coxsackievirus B species, Coxsackievirus C species, Porcine enterovirus species, Bovine enterovirus species, Hepatovirus species, Cardiovirus species, or combinations thereof.

In some embodiments, a method for simultaneous determination of the identity and quantity of a Picornavirus in a sample is provided, comprising (a) contacting the sample with a pair of oligonucleotide primers and a known quantity of a calibration polynucleotide comprising a calibration polynucleotide sequence; (b) simultaneously amplifying the DNA from at least one Picornavirus with the pair of oligonucleotide primers and amplifying nucleic acid from the calibration polynucleotide in the sample with the pair of oligonucleotide primers to obtain at least one Picornavirus identifying amplification product and at least one calibration polynucleotide amplification product; (c) subjecting the sample to molecular mass analysis using a mass spectrometer wherein the result of the molecular mass analysis comprises molecular mass and abundance data for the Picornavirus identifying amplification product and the calibration polynucleotide amplification product; and (d) distinguishing the Picornavirus identifying amplification product from the calibration polynucleotide amplification product by molecular mass analysis wherein the molecular mass of said Picornavirus identifying amplification product identifies at least one Picornavirus in the sample, and comparison of the abundance of the Picornavirus identifying amplification product and the calibration polynucleotide amplification product indicates the quantity of Picornavirus in the sample. In some embodiments, the pair of oligonucleotide primers hybridize with a DNA sequence corresponding to a RNA sequence of at least three Picornavirus family members and flank variable regions that vary between at least three Picornavirus family members. In other embodiments, the calibration polynucleotide sequence comprises the sequence of a standard sequence of a Picornavirus identifying amplification product further comprising the deletion of 2-8 consecutive nucleotide residues of the standard sequence in the calibration polynucleotide sequence. In still other embodiments, the calibration polynucleotide sequence comprises the sequence of a standard sequence of a Picornavirus identifying amplification product further comprising the insertion of 2-8 consecutive nucleotide residues in the standard sequence in the calibration polynucleotide sequence. In additional embodiments, the calibration polynucleotide sequence comprises at least 80%, at least 90%, or at least 95% sequence identity with a standard sequence of a picornavirus identifying amplification product. In certain embodiments, the calibration polynucleotide resides on a plasmid. In other embodiments, the molecular mass analysis comprises ESI-TOF molecular mass analysis.

In some embodiments, a multiplex polymerase chain reaction method for identifying a Picornavirus is provided comprising (a) providing a sample suspected of comprising one or more Picornavirus family members; (b) providing Picornavirus RNA from the sample; (c) providing DNA obtained from the RNA wherein the RNA comprises sequences encoding genes selected from the group of WTPV1, WTHRV14, WTCVB3, PCV305, PV5′HRV14, GENOME5UTR, GENOMEUTR, POLIO5UTR, POLIO3D, POLIO3C, COXA3D, COXA2C, COXB3D, COXB2C, COXB3C, COXC3D, HRVA3D, HRVA2C, HRVBVP1, HRVB2B, HAV, CARDIOVIRUS5UTR, CARDIOVIRUS1C, CARDIOVIRUS3D, CARDIOVIRUS3AB, THEILOVIRUS5UTR, THEILOVIRUS5UTR-1A, THEILOVIRUS2A-2B, THEILOVIRUS2C, and THEILOVIRUS3D genes; (d) amplifying the DNA to produce at least one amplification product using two or more oligonucleotide primer pairs; (e) determining the base composition of the at least one amplification product by mass spectrometry, wherein the base composition identifies the number of A residues, C residues, T residues, G residues, U residues, analogs thereof and mass tag residues thereof in the amplification product; and (f) comparing the base composition of the amplification product to calculated or measured base compositions of amplification products of one or more known Picornaviruses in a database with the proviso that sequencing of the amplification product is not used to identify the Picornavirus, wherein a match between the determined base composition and the calculated or measured base composition in the database identifies the genus, species or strain of the one or more picornavirus family members in the sample. In some embodiments, at least one forward member of the two or more primer pairs comprises at least 70%, at least 80%, at least 90%, at least 95%, or at least 100% sequence identity with a sequence selected from the group consisting of SEQ ID NOs: 2-30, 60-71, and 84-97, and at least one reverse member of the two or more primer pairs comprises at least 70% sequence identity with a sequence selected from the group consisting of SEQ ID NOs: 31-59, 72-83, and 98-111.

In certain embodiments, the amplifying is carried out in a single reaction vessel. In other embodiments, the amplifying is carried out in one or more primer pair specific reaction vessels. In still other embodiments, the one or more Picornavirus family members are identified in the sample, the identified family members comprising one or more of Enterovirus A species, Enterovirus B species, Enterovirus C species, Enterovirus D species, Poliovirus genus, Rhinovirus genus, Rhinovirus A species, Rhinovirus B species, Coxsackievirus genus, Picornavirus A species, Picornavirus B species, Picornavirus C species, Picornavirus species, Picornavirus species, Picornavirus species, Picornavirus species, or combinations thereof. In some embodiments, the mass spectrometry comprises ESI-TOF mass spectrometry.

### BRIEF DESCRIPTION OF THE DRAWINGS

The foregoing summary and detailed description is better understood when read in conjunction with the accompanying drawings which are included by way of example and not by way of limitation.

FIG. 1. Shows a process diagram illustrating one embodiment of the primer pair selection process.

FIG. 2. Shows a process diagram illustrating one embodiment of the primer pair validation process. Here select primers are shown meeting test criteria. Criteria include but are not limited to, the ability to amplify targeted viruses, the ability to exclude non-target species, the ability to not produce unexpected amplicons, the ability to not dimerize, the ability to have analytical limits of detection of ≦100 genomic copies/reaction, and the ability to differentiate amongst different target organisms.

FIG. 3A. Shows an example of mass spectra of amplification products of Enterovirus obtained by amplification of nucleic acid of Enterovirus calibrant with primer pair number 3759.

FIG. 3B. Shows an example of mass spectra of amplification products of Enterovirus obtained by amplification of nucleic acid of Enterovirus calibrant with primer pair number 3758.

FIG. 3C. Shows an example of mass spectra of amplification products of Enterovirus obtained by amplification of nucleic acid of Enterovirus calibrant with primer pair number 3760.

FIG. 3D. Shows an example of mass spectra of amplification products of Enterovirus obtained by amplification of nucleic acid of Enterovirus calibrant with primer pair number 3761.

FIG. 3E. Shows an example of mass spectra of amplification products of Rhinovirus obtained by amplification of nucleic acid of Rhinovirus calibrant with primer pair number 3763.

FIG. 3F. Shows an example of mass spectra of amplification products of Rhinovirus obtained by amplification of nucleic acid of Rhinovirus calibrant with primer pair number 3764.

FIG. 3G. Shows an example of mass spectra of amplification products of Rhinovirus obtained by amplification of nucleic acid of Rhinovirus calibrant with primer pair number 3764.

FIG. 4. Shows a process diagram illustrating an embodiment of the calibration method.

FIG. 5. Shows a representation of a PV5′HRV14 construct. The construct consists of HRV14 sequences with a substitution of the 5′NCR region with sequences from WTPV1. A PCV305 construct is similar except that the primary region of the genomic sequence is derived from WTPV1 while the 5′NCR region is derived from WTCVB3.

FIG. 6. Shows the theoretical distribution of base compositions of Enterovirus and Rhinovirus species members based on sequence data for primer pair 3758.

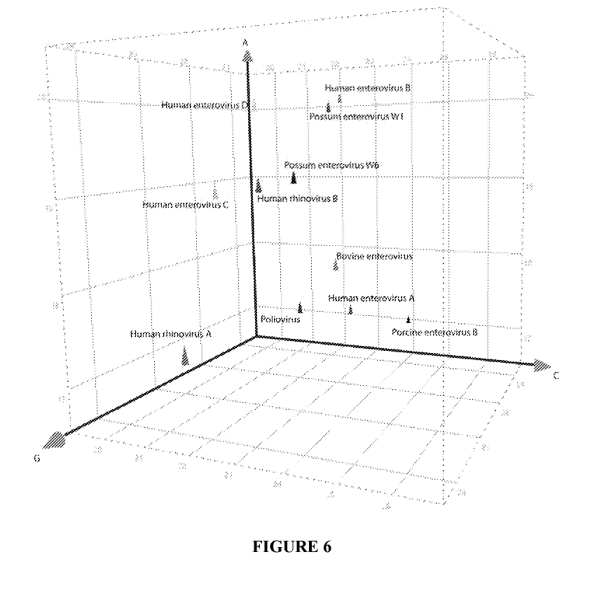

FIG. 7. Shows Hepatovirus primer testing results with variant calibrant titrations at 2× dilutions:5000 copies to zero copies.

FIG. 8. Shows Hepatovirus testing results with detection of four different ATCC HAV stocks. More specifically, the top panel shows 2× limiting dilutions of the RNA calibrant standard tested against one of the broad primers (PP3043) from Tables 9 and 10. Based on the detection of the calibrant, this primer was sensitive down to 10 copies of input RNA per well. While the calibrant was detected at 5 copies as well, there was a strong primer dimer, indicating weaker binding to the target. The bottom panel shows detection of four different ATCC HAV stocks (VR: 1541, 2089, 2092 and 2266) using two of the primer pairs, PP3035 and 3043.

FIG. 9. Block diagram showing a representative system.

### DETAILED DESCRIPTION OF EMBODIMENTS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the present invention, the following terminology and grammatical variants will be used in accordance with the definitions set forth below.

As used herein, the term “about” means encompassing plus or minus 10%. For example, about 200 nucleotides refers to a range encompassing between 180 and 220 nucleotides.

As used herein, the term “amplicon” or “bioagent identifying amplicon” refers to a nucleic acid generated using the primer pairs described herein. The amplicon is typically double stranded DNA; however, it may be RNA and/or DNA:RNA. In some embodiments, the amplicon comprises DNA complementary to Picornavirus RNA. In some embodiments, the amplicon comprises DNA complementary to Enterovirus RNA. In some embodiments, the amplicon comprises DNA complementary to Rhinovirus RNA. In some embodiments, the amplicon comprises DNA complementary to Hepatovirus RNA. In some embodiments, the amplicon comprises DNA complementary to Cardiovirus RNA. In some embodiments, the amplicon comprises the sequences of the conserved regions/primer pairs and the intervening variable region. As discussed herein, primer pairs are configured to generate amplicons from two or more bioagents. As such, the base composition of any given amplicon may include the primer pair, the complement of the primer pair, the conserved regions and the variable region from the bioagent that was amplified to generate the amplicon. One skilled in the art understands that the incorporation of the designed primer pair sequences into an amplicon may replace the native viral sequences at the primer binding site, and complement thereof. After amplification of the target region using the primers the resultant amplicons having the primer sequences are used to generate the molecular mass data. Such is accounted for when identifying one or more bioagents using any particular primer pair. The amplicon further comprises a length that is compatible with mass spectrometry analysis. Bioagent identifying amplicons generate base compositions that are preferably unique to the identity of a bioagent.

Amplicons typically comprise from about 45 to about 200 consecutive nucleobases (i.e., from about 45 to about 200 linked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One ordinarily skilled in the art will further appreciate that the above range is not an absolute limit to the length of an amplicon, but instead represents a preferred length range. Amplicons lengths falling outside of this range are also included herein so long as the amplicon is amenable to calculation of a base composition signature as herein described.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “base composition” refers to the number of each residue comprised in an amplicon or other nucleic acid, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill et al.), an acyclic nucleoside analog containing 5-nitroindazole (Van Aerschot et al., Nucleosides and Nucleotides, 1995, 14, 1053-1056), the purine analog 1-(2-deoxy-.beta.-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C. In some embodiments, the non-natural nucleosides used herein include 5-propynyluracil, 5-propynylcytosine and inosine. Herein the base composition for an unmodified DNA amplicon is notated as A_{w}GₓC_{y}T_{z}, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. A match between the calculated base composition and a single database entry reveals the identity of the bioagent.

As used herein, a “base composition probability cloud” is a representation of the diversity in base composition resulting from a variation in sequence that occurs among different isolates of a given species, family or genus. Base composition calculations for a plurality of amplicons are mapped on a pseudo four-dimensional plot. Related members in a family, genus or species typically cluster within this plot, forming a base composition probability cloud.

As used herein, the term “base composition signature” refers to the base composition generated by any one particular amplicon.

As used herein, a “bioagent” means any microorganism or infectious substance, or any naturally occurring, bioengineered or synthesized component of any such microorganism or infectious substance or any nucleic acid derived from any such microorganism or infectious substance. Those of ordinary skill in the art will understand fully what is meant by the term bioagent given the instant disclosure. Still, a non-exhaustive list of bioagents includes: cells, cell lines, human clinical samples, mammalian blood samples, cell cultures, bacterial cells, viruses, viroids, fungi, protists, parasites, rickettsiae, protozoa, animals, mammals or humans. Samples may be alive, non-replicating or dead or in a vegetative state (for example, vegetative bacteria or spores). Preferably, the bioagent is a virus or a nucleic acid derived therefrom. More preferably, the bioagent is a member of the Picornaviridae family (i.e., a picornavirus bioagent). More preferably still the bioagent is a rhinovirus or a enterovirus of the subgenera polioviruses, coxsackieviruses (groups A, B and C), echoviruses, cardiovirus, hepatovirus, or the like.

As used herein, a “bioagent division” is defined as group of bioagents above the species level and includes but is not limited to, orders, families, genus, classes, clades, genera or other such groupings of bioagents above the species level.

As used herein, “broad range survey primers” are intelligent primers designed to identify an unknown bioagent as a member of a particular biological division (e.g., an order, family, class, clade, or genus). However, in some cases the broad range survey primers are also able to identify unknown bioagents at the species or sub-species level. As used herein, “division-wide primers” are intelligent primers designed to identify a bioagent at the species level and “drill-down” primers are intelligent primers designed to identify a bioagent at the sub-species level. As used herein, the “sub-species” level of identification includes, but is not limited to, strains, subtypes, variants, and isolates. Preferably, and without limitation, the family is Picornaviridae the genus includes members of Enterovirus genus including Poliovirus, Coxsackievirus, and Echovirus; human Rhinovirus genus; human Hepatovirus genus including Hepatitis A, Cardiovirus genus, and others. Drill-down primers are not always required for identification at the sub-species level because broad range survey intelligent primers may, in some cases provide sufficient identification resolution to accomplishing this identification objective.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “conserved region” in the context of nucleic acids refers to a nucleobase sequence (e.g., a subsequence of a nucleic acid, etc.) that is the same or similar in two or more different regions or segments of a given nucleic acid molecule (e.g., an intramolecular conserved region), or that is the same or similar in two or more different nucleic acid molecules (e.g., an intermolecular conserved region). To illustrate, a conserved region may be present in two or more different taxonomic ranks (e.g., two or more different genera, two or more different species, two or more different subspecies, and the like) or in two or more different nucleic acid molecules from the same organism. To further illustrate, in certain embodiments, nucleic acids comprising at least one conserved region typically have between about 70%-100%, between about 80-100%, between about 90-100%, between about 95-100%, or between about 99-100% sequence identity in that conserved region.

The term “correlates” refers to establishing a relationship between two or more things. In certain embodiments, for example, detected molecular masses of one or more amplicons indicate the presence or identity of a given bioagent in a sample. In some embodiments, base compositions are calculated or otherwise determined from the detected molecular masses of amplicons, which base compositions indicate the presence or identity of a given bioagent in a sample.

As used herein, in some embodiments the term “database” is used to refer to a collection of base composition molecular mass data. In other embodiments the term “database” is used to refer to a collection of base composition data. The base composition data in the database is indexed to bioagents and to primer pairs. The base composition data reported in the database comprises the number of each nucleoside in an amplicon that would be generated for each bioagent using each primer. The database can be populated by empirical data. In this aspect of populating the database, a bioagent is selected and a primer pair is used to generate an amplicon. The amplicon's molecular mass is determined using a mass spectrometer and the base composition calculated therefrom without sequencing i.e., without determining the linear sequence of nucleobases comprising the amplicon. Note that base composition entries in the database may be derived from sequencing data (i.e., in the art), but the base composition of the amplicon to be identified is determined without sequencing the amplicon. An entry in the database is made to associate correlate the base composition with the bioagent and the primer pair used. The database may also be populated using other databases comprising bioagent information. For example, using the GenBank database it is possible to perform electronic PCR using an electronic representation of a primer pair. This in silico method may provide the base composition for any or all selected bioagent(s) stored in the GenBank database. The information may then be used to populate the base composition database as described above. A base composition database can be in silico, a written table, a reference book, a spreadsheet or any form generally amenable to databases. Preferably, it is in silico on computer readable media.

The term “detect”, “detecting” or “detection” refers to an act of determining the existence or presence of one or more targets (e.g., viral nucleic acids, amplicons, etc.) in a sample.

As used herein, the term “etiology” refers to the causes or origins, of diseases or abnormal physiological conditions.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to nucleic acid sequences that are not found naturally associated with the gene sequences in the chromosome or are associated with portions of the chromosome not found in nature (e.g., genes expressed in loci where the gene is not normally expressed).

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. In context of the present invention, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers of the present invention, sequence identity is properly determined when the alignment is designated as Plus/Plus. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “housekeeping gene” or “core viral gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, “intelligent primers” or “primers” or “primer pairs” are oligonucleotides that are designed to bind to conserved sequence regions of two or more bioagent nucleic acid to generate bioagent identifying amplicons. In some embodiments, the bound primers flank an intervening variable region between the conserved binding sequences. Upon amplification, the primer pairs yield amplicons i.e., amplification products that provide base composition variability between the two or more bioagents. The variability of the base compositions allows for the identification of one or more individual bioagents from, e.g., two or more bioagents based on the base composition distinctions. The primer pairs are also configured to generate amplicons amenable to molecular mass analysis. Primer pair nomenclature, as used herein, includes naming a reference sequence. For example, the forward primer for primer pair number 3758 is named GENOME5UTR_NC001472-1-7389_{—}445_{—}463_F. The reference sequence that this primer is referring to is GenBank Accession No: NC_{—}001472 (first entered Aug. 1, 2000) (SEQ ID NO: 1). This primer is the forward primer of the pair (as denoted by “_F”) and it hybridizes with residues 445-463 of the reference sequence (445_{—}463), of the referenced Human Enterovirus B. The primer pairs are selected and configured in some embodiments, however, to hybridize with two or more bioagents. So, the nomenclature used is merely to provide a reference sequence, and not to indicate that the primers hybridize with and generate a bioagent identifying amplicon only from the reference sequence. Further, the sequences of the primer members of the primer pairs are not necessarily fully complementary to the conserved region of the reference bioagent. Rather, the sequences are designed to be “best fit” amongst a plurality of bioagents at these conserved binding sequences. Therefore, the primer members of the primer pairs have substantial complementarity with the conserved regions of the bioagents, including the reference bioagent.

As used herein, the term “molecular mass” refers to the mass of a compound as determined using mass spectrometry, specifically ESI-MS. Herein, the compound is preferably a nucleic acid, more preferably a double stranded nucleic acid, still more preferably a double stranded DNA nucleic acid and is most preferably an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5 (carboxyhydroxyl-methyl) uracil, 5-fluorouracil, 5 bromouracil, 5-carboxymethylaminomethyl 2 thiouracil, 5 carboxymethyl-aminomethyluracil, dihydrouracil, inosine, N6 isopentenyladenine, 1 methyladenine, 1-methylpseudo-uracil, 1 methylguanine, 1 methylinosine, 2,2-dimethyl-guanine, 2 methyladenine, 2 methylguanine, 3-methyl-cytosine, 5 methylcytosine, N6 methyladenine, 7 methylguanine, 5 methylaminomethyluracil, 5-methoxy-amino-methyl 2 thiouracil, beta D mannosylqueosine, 5′ methoxycarbonylmethyluracil, 5 methoxyuracil, 2 methylthio N6 isopentenyladenine, uracil 5 oxyacetic acid methylester, uracil 5 oxyacetic acid, oxybutoxosine, pseudouracil, queosine, 2 thiocytosine, 5-methyl-2 thiouracil, 2-thiouracil, 4 thiouracil, 5-methyluracil, N-uracil 5 oxyacetic acid methylester, uracil 5 oxyacetic acid, pseudouracil, queosine, 2-thiocytosine, and 2,6 diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No.

4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

The term “probe nucleic acid” or “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using a probe under sufficiently stringent hybridization conditions permits the selective detection of a specific target sequence. The term “hybridizing region” refers to that region of a nucleic acid that is exactly or substantially complementary to, and therefore capable of hybridizing to, the target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support. A probe is generally included in a nucleic acid that comprises one or more labels (e.g., donor moieties, acceptor moieties, and/or quencher moieties), such as a 5′-nuclease probe, a hybridization probe, a fluorescent resonance energy transfer (FRET) probe, a hairpin probe, or a molecular beacon, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially or substantially complementary to one another); stable hybridization complexes may contain mismatched bases or unmatched bases. Modification of the stringent conditions may be necessary to permit a stable hybridization complex with one or more base pair mismatches or unmatched bases. Sambrook et al., Molecular Cloning: A Laboratory Manual, 3rd Ed., Cold Spring Harbor Laboratory Press, Cold Spring Harbor, N.Y. (2001), which is incorporated by reference, provides guidance for suitable modification. Stability of the target/probe hybridization complex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids.

In some embodiments of the invention, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein a “sample” refers to anything capable of being analyzed by the methods provided herein. In some embodiments, the sample comprises or is suspected one or more nucleic acids capable of analysis by the methods. Preferably, the samples comprise nucleic acids (e.g., RNA, cDNAs, etc.) from one or more members of the Picornaviridae family. Samples can include, for example, evidence from a crime scene, blood, blood stains, semen, semen stains, bone, teeth, hair saliva, urine, feces, fingernails, muscle tissue, cigarettes, stamps, envelopes, dandruff, fingerprints, personal items, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As is used herein, the term “single primer pair identification” means that one or more bioagents can be identified using a single primer pair. A base composition signature for an amplicon may singly identify one or more bioagents.

As used herein, a “sub-species characteristic” is a genetic characteristic that provides the means to distinguish two members of the same bioagent species. For example, one viral strain may be distinguished from another viral strain of the same species by possessing a genetic change (e.g., for example, a nucleotide deletion, addition or substitution) in one of the viral genes, such as the RNA-dependent RNA polymerase.

As used herein, in some embodiments the term “substantial complementarity” means that a primer member of a primer pair comprises between about 70%-100%, or between about 80-100%, or between about 90-100%, or between about 95-100%, or between about 99-100% complementarity with the conserved binding sequence of a nucleic acid from a given bioagent. Similarly, the primer pairs provided herein may comprise between about 70%-100%, or between about 80-100%, or between about 90-100%, or between about 95-100% identity, or between about 99-100% sequence identity with the primer pairs disclosed in Tables 2, 7, 8, and 9. These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. In some embodiments, any oligonucleotide primer pair may have one or both primers with less then 70% sequence homology with a corresponding member of any of the primer pairs of Tables 2, 7, 8, and 9 if the primer pair has the capability of producing an amplification product corresponding to the desired picornavirus identifying amplicon.

A “system” in the context of analytical instrumentation refers a group of objects and/or devices that form a network for performing a desired objective.

As used herein, “triangulation identification” means the use of more than one primer pair to generate a corresponding amplicon for identification of a bioagent. The more than one primer pair can be used in individual wells or vessels or in a multiplex PCR assay. Alternatively, PCR reactions may be carried out in single wells or vessels comprising a different primer pair in each well or vessel. Following amplification the amplicons are pooled into a single well or container which is then subjected to molecular mass analysis. The combination of pooled amplicons can be chosen such that the expected ranges of molecular masses of individual amplicons are not overlapping and thus will not complicate identification of signals. Triangulation is a process of elimination, wherein a first primer pair identifies that an unknown bioagent may be one of a group of bioagents. Subsequent primer pairs are used in triangulation identification to further refine the identity of the bioagent amongst the subset of possibilities generated with the earlier primer pair. Triangulation identification is complete when the identity of the bioagent is determined. The triangulation identification process may also be used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected compositions from the B. anthracis genome would suggest a genetic engineering event.

As used herein, the term “unknown bioagent” can mean, for example: (i) a bioagent whose existence is not known (for example, the SARS coronavirus was unknown prior to April 2003) and/or (ii) a bioagent whose existence is known (such as the well known bacterial species Staphylococcus aureus for example) but which is not known to be in a sample to be analyzed. For example, if the method for identification of coronaviruses disclosed in commonly owned U.S. patent Ser. No. 10/829,826 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. patent Ser. No. 10/829,826 was to be employed subsequent to April 2003 to identify the SARS coronavirus in a clinical sample, the second meaning (ii) of “unknown” bioagent would apply because the SARS coronavirus became known to science subsequent to April 2003 because it was not known what bioagent was present in the sample.

As used herein, the term “variable region” is used to describe a region that falls between any one primer pair described herein. The region possesses distinct base compositions between at least two bioagents, such that at least one bioagent can be identified at the family, genus, species or sub-species level. The degree of variability between the at least two bioagents need only be sufficient to allow for identification using mass spectrometry analysis, as described herein.

As used herein, “viral nucleic acid” includes, but is not limited to, DNA, RNA, or DNA that has been obtained from viral RNA, such as, for example, by performing a reverse transcription reaction. Viral RNA can either be single-stranded (of positive or negative polarity) or double-stranded.

As used herein, a “wobble base” is a variation in a codon found at the third nucleotide position of a DNA triplet. Variations in conserved regions of sequence are often found at the third nucleotide position due to redundancy in the amino acid code.

Provided herein are methods, compositions, kits, and related systems for the detection and identification of bioagents using bioagent identifying amplicons. In overview, primers may be selected to hybridize to conserved sequence regions of nucleic acids derived from a bioagent and which bracket variable sequence regions to yield a bioagent identifying amplicon which can be amplified and which is amenable to molecular mass determination. The molecular mass is typically converted to a base composition, which indicates the number of each nucleotide in the amplicon. The molecular mass or corresponding base composition signature of the amplicon is then typically queried against a database of molecular masses or base composition signatures indexed to bioagents and to the primer pair used to generate the amplicon. A match of the measured base composition to a database entry base composition associates the sample bioagent to an indexed bioagent in the database. Thus, the identity of the unknown bioagent is determined in certain embodiments. Prior knowledge of the unknown bioagent is not necessary. In some instances, the measured base composition associates with more than one database entry base composition. Thus, a second/subsequent primer pair is generally used to generate an amplicon, and its measured base composition is similarly compared to the database to determine its identity in triangulation identification. Furthermore, the methods and other aspects of the invention can be applied to rapid parallel multiplex analyses, the results of which can be employed in a triangulation identification strategy. The present invention provides rapid throughput and does not require nucleic acid sequencing of the amplified target sequence for bioagent detection and identification.

Since genetic data provide the underlying basis for identification of bioagents, it is generally necessary to select segments or regions of nucleic acids which provide sufficient variability to distinguish individual bioagents and whose molecular mass is amenable to molecular mass determination.

Unlike bacterial genomes, which exhibit conservation of numerous genes (i.e. housekeeping genes) across all organisms, viruses typically do not share a single gene that is essential and conserved among all virus families. Therefore, viral identification is generally achieved within smaller groups of related viruses, such as members of a particular virus family or genus. For example, RNA-dependent RNA polymerase (RdRp) is present in all single-stranded RNA viruses and can be used for broad priming as well as resolution within the virus family.

In some embodiments, at least one viral nucleic acid segment is amplified in the process of identifying the bioagent. Thus, the nucleic acid segments that can be amplified by the primers disclosed herein and that provide sufficient variability to distinguish individual bioagents and whose molecular masses are amenable to molecular mass determination are herein described as bioagent identifying amplicons. In certain embodiments, picornavirus bioagents are identified via amplicons generated with the primers described herein using methods of detection other than molecular mass-based detection, such as real-time PCR (e.g., using 5′-nuclease probes, hairpin probes, hybridization probes, nucleic acid binding dyes, or the like) or other approaches known to persons of skill in the art.

In some embodiments, it is the combination of the portions of the bioagent nucleic acid segment to which the primers hybridize (hybridization sites) and the variable region between the primer hybridization sites that comprises the bioagent identifying amplicon.

In certain embodiments, bioagent identifying amplicons amenable to molecular mass determination which are produced by the primers described herein are either of a length, size or mass compatible with the particular mode of molecular mass determination or compatible with a means of providing a predictable fragmentation pattern in order to obtain predictable fragments of a length compatible with the particular mode of molecular mass determination. Such means of providing a predictable fragmentation pattern of an amplicon include, but are not limited to, cleavage with restriction enzymes or cleavage primers, sonication or other means of fragmentation. Thus, in some embodiments, bioagent identifying amplicons are larger than 200 nucleobases and are amenable to molecular mass determination following restriction digestion. Methods of using restriction enzymes and cleavage primers are well known to those with ordinary skill in the art.

In some embodiments, amplicons corresponding to bioagent identifying amplicons are obtained using the polymerase chain reaction (PCR) which is a routine method to those with ordinary skill in the molecular biology arts. Other amplification methods may be used such as ligase chain reaction (LCR), low-stringency single primer PCR, and multiple strand displacement amplification (MDA). These methods are also known to those with ordinary skill. (Michael, S F., Biotechniques (1994), 16:411-412 and Dean et al., Proc. Natl. Acad. Sci. U.S.A. (2002), 99, 5261-5266).

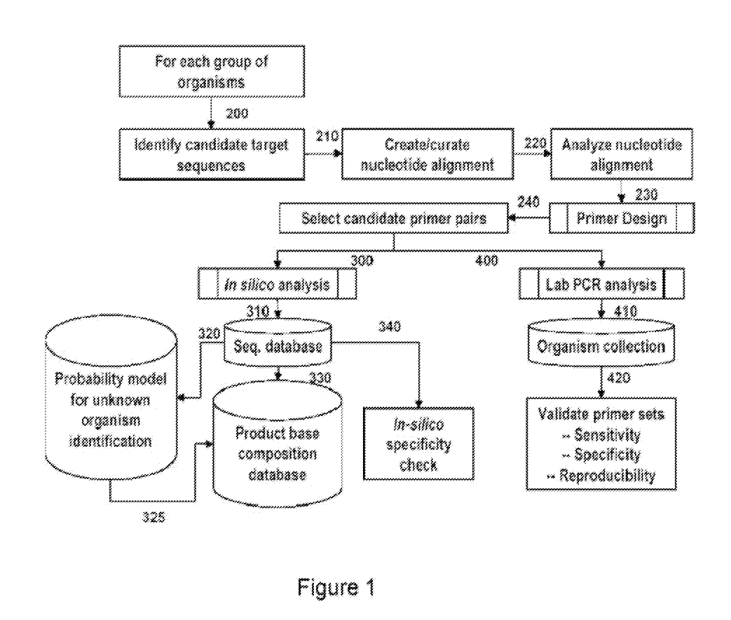

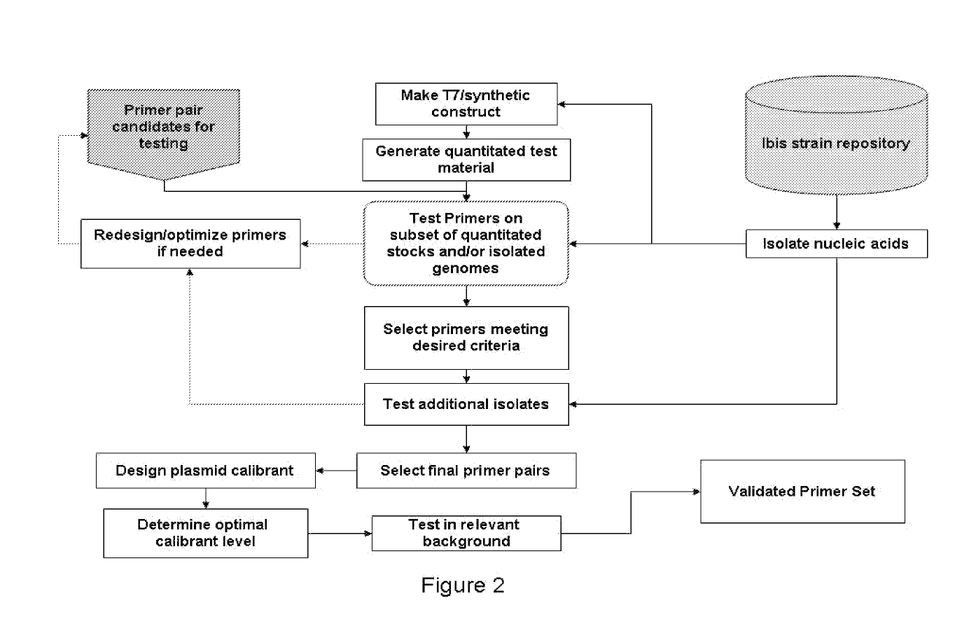

One embodiment of a process flow diagram used for primer selection and validation process is depicted in FIGS. 1 and 2. For each group of organisms, candidate target sequences are identified (200) from which nucleotide alignments are created (210) and analyzed (220). Primers are then configured by selecting priming regions (230) to facilitate the selection of candidate primer pairs (240). The primer pair sequence is typically a “best fit” amongst the aligned sequences, such that the primer pair sequence may or may not be fully complementary to the hybridization region on any one of the bioagents in the alignment. Thus, best fit primer pair sequences are those with sufficient complementarity with two or more bioagents to hybridize with the two or more bioagents and generate an amplicon. The primer pairs are then subjected to in silico analysis by electronic PCR (ePCR) (300) wherein bioagent identifying amplicons are obtained from sequence databases such as GenBank or other sequence collections (310) and tested for specificity in silico (320). Bioagent identifying amplicons obtained from ePCR of GenBank sequences (310) may also be analyzed by a probability model which predicts the capability of a given amplicon to identify unknown bioagents. Preferably, the base compositions of amplicons with favorable probability scores are then stored in a base composition database (325). Alternatively, base compositions of the bioagent identifying amplicons obtained from the primers and GenBank sequences are directly entered into the base composition database (330). Candidate primer pairs (240) are validated by in vitro amplification by a method such as PCR analysis (400) of nucleic acid from a collection of organisms (410). Amplicons thus obtained are analyzed to confirm the sensitivity, specificity and reproducibility of the primers used to obtain the amplicons (420).

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The primers typically are employed as compositions for use in methods for identification of viral bioagents as follows: a primer pair composition is contacted with nucleic acid (such as, for example, DNA from a DNA virus, or DNA reverse transcribed from the RNA of an RNA virus) of an unknown viral bioagent. The nucleic acid is then amplified by a nucleic acid amplification technique, such as PCR for example, to obtain an amplicon that represents a bioagent identifying amplicon. The molecular mass of the strands of the double-stranded amplicon is determined by a molecular mass measurement technique such as mass spectrometry, for example. Preferably the two strands of the double-stranded amplicon are separated during the ionization process; however, they may be separated prior to mass spectrometry measurement. In some embodiments, the mass spectrometer is electrospray Fourier transform ion cyclotron resonance mass spectrometry (ESI-FTICR-MS) or electrospray time of flight mass spectrometry (ESI-TOF-MS). A list of possible base compositions may be generated for the molecular mass value obtained for each strand and the choice of the base composition from the list is facilitated by matching the base composition of one strand with a complementary base composition of the other strand. The measured molecular mass or base composition calculated therefrom is then compared with a database of molecular masses or base compositions indexed to primer pairs and to known viral bioagents. A match between the measured molecular mass or base composition of the amplicon and the database molecular mass or base composition for that indexed primer pair will correlate the measured molecular mass or base composition with an indexed viral bioagent, thus identifying the unknown bioagent. In some embodiments, the primer pair used is at least one of the primer pairs of Table 2, 7, 8, and/or 9. In some embodiments, the method is repeated using a different primer pair to resolve possible ambiguities in the identification process or to improve the confidence level for the identification assignment (triangulation identification).

In some embodiments, a bioagent identifying amplicon may be produced using only a single primer (either the forward or reverse primer of any given primer pair), provided an appropriate amplification method is chosen, such as, for example, low stringency single primer PCR (LSSP-PCR).

In some embodiments, the oligonucleotide primers are broad range survey primers which hybridize to conserved regions of nucleic acid encoding, e.g., the PB1 gene or the NUC gene, a gene that is common to all known enteroviruses, though the sequences vary. The broad range primer may identify the unknown bioagent, depending on which bioagent is in the sample. In other cases, the molecular mass or base composition of an amplicon does not provide sufficient resolution to identify the unknown bioagent as any one viral bioagent at or below the species level. These cases generally benefit from further analysis of one or more amplicons generated from at least one additional broad range survey primer pair or from at least one additional division-wide primer pair, or from at least one additional drill-down primer pair. Identification of sub-species characteristics may be needed for determining proper clinical treatment of viral infections, or in rapidly responding to an outbreak of a new viral strain to prevent massive epidemic or pandemic.

In some embodiments, the primers used for amplification hybridize to and amplify genomic DNA, DNA of bacterial plasmids, DNA of DNA viruses or DNA reverse transcribed from RNA of an RNA virus. Among other things, identification of non-viral nucleic acids or combinations of viral and non-viral nucleic acids is useful for detecting bioengineered bioagents.

In some embodiments, the primers used for amplification hybridize directly to viral RNA and act as reverse transcription primers for obtaining DNA from direct amplification of viral RNA. Methods of amplifying RNA to produce cDNA using reverse transcriptase are well known to those with ordinary skill in the art and can be routinely established without undue experimentation.

One with ordinary skill in the art of design of amplification primers will recognize that a given primer need not hybridize with 100% complementarity in order to effectively prime the synthesis of a complementary nucleic acid strand in an amplification reaction. Primer pair sequences may be a “best fit” amongst the aligned bioagent sequences, thus not be fully complementary to the hybridization region on any one of the bioagents in the alignment. Moreover, a primer may hybridize over one or more segments such that intervening or adjacent segments are not involved in the hybridization event (e.g., for example, a loop structure or a hairpin structure). The primers may comprise at least 70%, at least 75%, at least 80%, at least 85%, at least 90%, at least 95% or at least 99% sequence identity with any of the primers listed in Tables 2, 7, 8, and 9. Thus, in some embodiments, an extent of variation of 70% to 100%, or any range falling within, of the sequence identity is possible relative to the specific primer sequences disclosed herein. To illustrate, determination of sequence identity is described in the following example: a primer 20 nucleobases in length which is identical to another 20 nucleobase primer having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Percent identity need not be a whole number, for example when a 28 consecutive nucleobase primer is completely identical to a 31 consecutive nucleobase primer (28/31=0.9032 or 90.3% identical).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of viral nucleic acid, is between about 70% and about 80%. In other embodiments, homology, sequence identity or complementarity, is between about 80% and about 90%. In yet other embodiments, homology, sequence identity or complementarity, is at least 90%, at least 92%, at least 94%, at least 95%, at least 96%, at least 97%, at least 98%, at least 99% or is 100%.

In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range falling within) sequence identity with the primer sequences specifically disclosed herein.

One with ordinary skill is able to calculate percent sequence identity or percent sequence homology and is able to determine, without undue experimentation, the effects of variation of primer sequence identity on the function of the primer in its role in priming synthesis of a complementary strand of nucleic acid for production of an amplicon of a corresponding bioagent identifying amplicon.

In some embodiments, the oligonucleotide primers are 13 to 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of, e.g., Taq DNA polymerase (Magnuson et al., Biotechniques, 1996, 21, 700-709), an occurrence which may lead to ambiguous results arising from molecular mass analysis.

Primers may contain one or more universal bases. Because any variation (due to codon wobble in the third position) in the conserved regions among species is likely to occur in the third position of a DNA (or RNA) triplet, oligonucleotide primers can be designed such that the nucleotide corresponding to this position is a base which can bind to more than one nucleotide, referred to herein as a “universal nucleobase.” For example, under this “wobble” pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003), the degenerate nucleotides dP or dK (Hill et al.), an acyclic nucleoside analog containing 5-nitroindazole (Van Aerschot et al., Nucleosides and Nucleotides, 1995, 14, 1053-1056) or the purine analog 1-(2-deoxy-.beta.-D-ribofuranosyl)-imidazole-4-carboxamide (Sala et al., Nucl. Acids Res., 1996, 24, 3302-3306).

In some embodiments, to compensate for weaker binding by the wobble base, the oligonucleotide primers are configured such that the first and second positions of each triplet are occupied by nucleotide analogs which bind with greater affinity than the unmodified nucleotide. Examples of these analogs include, but are not limited to, 2,6-diaminopurine which binds to thymine, 5-propynyluracil which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is commonly owned and incorporated herein by reference in its entirety. Propynylated primers are described in U.S. Pre-Grant Publication No. 2003-0170682; also commonly owned and incorporated herein by reference in its entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, to enable broad priming of rapidly evolving RNA viruses, primer hybridization is enhanced using primers and probes containing 5-propynyl deoxy-cytidine and deoxy-thymidine nucleotides. These modified primers offer increased affinity and base pairing selectivity.

In some embodiments, non-template primer tags are used to increase the melting temperature (Tₘ) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a possible source of ambiguity in determination of base composition of amplicons. Addition of mass-modifying tags to certain nucleobases of a given primer will result in simplification of de novo determination of base composition of a given bioagent identifying amplicon from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹³N and ¹³C.

In some embodiments, the molecular mass of a given bioagent identifying amplicon is determined by mass spectrometry. Mass spectrometry is intrinsically a parallel detection scheme without the need for radioactive or fluorescent labels, since every amplicon is identified by its molecular mass. The current state of the art in mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplicons using one of a variety of ionization techniques to convert the sample to the gas phase. These ionization methods include, but are not limited to, electrospray ionization (ESI), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, assignment of previously unobserved base compositions (also known as “true unknown base compositions”) to a given phylogeny can be accomplished via the use of pattern classifier model algorithms. Base compositions, like sequences, may vary slightly from strain to strain within species, for example. In some embodiments, the pattern classifier model is the mutational probability model. In other embodiments, the pattern classifier is the polytope model. A polytope model is the mutational probability model that incorporates both the restrictions among strains and position dependence of a given nucleobase within a triplet. In certain embodiments, a polytope pattern classifier is used to classify a test or unknown organism according to its amplicon base composition.

In some embodiments, it is possible to manage this diversity by building “base composition probability clouds” around the composition constraints for each species. A “pseudo four-dimensional plot” may be used to visualize the concept of base composition probability clouds. Optimal primer design typically involves an optimal choice of bioagent identifying amplicons and maximizes the separation between the base composition signatures of individual bioagents. Areas where clouds overlap generally indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of an unknown bioagent whose assigned base composition was not previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

Provided herein is bioagent classifying information at a level sufficient to identify a given bioagent. Furthermore, the process of determining a previously unknown base composition for a given bioagent (for example, in a case where sequence information is unavailable) has utility by providing additional bioagent indexing information with which to populate base composition databases. The process of future bioagent identification is thus improved as additional base composition signature indexes become available in base composition databases.

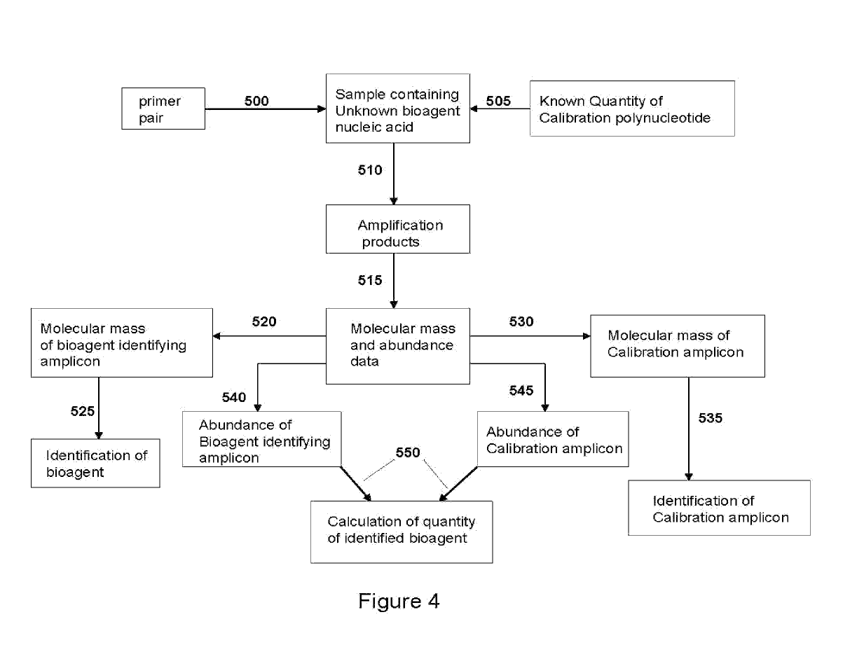

In some embodiments, the identity and quantity of an unknown bioagent may be determined using the process illustrated in FIG. 4. Primers (500) and a known quantity of a calibration polynucleotide (505) are added to a sample containing nucleic acid of an unknown bioagent. The total nucleic acid in the sample is then subjected to an amplification reaction (510) to obtain amplicons. The molecular masses of amplicons are determined (515) from which are obtained molecular mass and abundance data. The molecular mass of the bioagent identifying amplicon (520) provides for its identification (525) and the molecular mass of the calibration amplicon obtained from the calibration polynucleotide (530) provides for its quantification (535). The abundance data of the bioagent identifying amplicon is recorded (540) and the abundance data for the calibration data is recorded (545), both of which are used in a calculation (550) which determines the quantity of unknown bioagent in the sample.

In certain embodiments, a sample comprising an unknown bioagent is contacted with a primer pair which amplifies the nucleic acid from the bioagent, and a known quantity of a polynucleotide that comprises a calibration sequence. The rate of amplification is reasonably assumed to be similar for the nucleic acid of the bioagent and for the calibration sequence. The amplification reaction then produces two amplicons: a bioagent identifying amplicon and a calibration amplicon. The bioagent identifying amplicon and the calibration amplicon are distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent by base composition analysis. The abundance data enables calculation of the quantity of the bioagent, based on the knowledge of the quantity of calibration polynucleotide contacted with the sample.

In some embodiments, construction of a standard curve in which the amount of calibration or calibrant polynucleotide spiked into the sample is varied provides additional resolution and improved confidence for the determination of the quantity of bioagent in the sample. The use of standard curves for analytical determination of molecular quantities is well known to one with ordinary skill and can be performed without undue experimentation. Alternatively, the calibration polynucleotide can be amplified in its own PCR reaction vessel or vessels under the same conditions as the bioagent. A standard curve may be prepared there from, and the relative abundance of the bioagent determined by methods such as linear regression. In some embodiments, multiplex amplification is performed where multiple bioagent identifying amplicons are amplified with multiple primer pairs which also amplify the corresponding standard calibration sequences. In this or other embodiments, the standard calibration sequences are optionally included within a single construct (preferably a vector) which functions as the calibration polynucleotide. Competitive PCR, quantitative PCR, quantitative competitive PCR, multiplex and calibration polynucleotides are all methods and materials well known to those ordinarily skilled in the art and can be performed without undue experimentation.

In some embodiments, the calibrant polynucleotide is used as an internal positive control to confirm that amplification conditions and subsequent analysis steps are successful in producing a measurable amplicon. Even in the absence of copies of the genome of a bioagent, the calibration polynucleotide should give rise to a calibration amplicon. Failure to produce a measurable calibration amplicon indicates a failure of amplification or subsequent analysis step such as amplicon purification or molecular mass determination. Reaching a conclusion that such failures have occurred is, in itself, a useful event. In some embodiments, the calibration sequence is comprised of DNA. In some embodiments, the calibration sequence is comprised of RNA.

In some embodiments, a calibration sequence is inserted into a vector which then functions as the calibration polynucleotide. In some embodiments, more than one calibration sequence is inserted into the vector that functions as the calibration polynucleotide. Such a calibration polynucleotide is herein termed a “combination calibration polynucleotide.” The process of inserting polynucleotides into vectors is routine to those skilled in the art, and may be accomplished without undue experimentation. Thus, it should be recognized that the calibration method should not be limited to the embodiments described herein. The calibration method can be applied for determination of the quantity of any bioagent identifying amplicon when an appropriate standard calibrant polynucleotide sequence is designed and used.

The process of choosing an appropriate vector for insertion of a calibrant is also a routine operation that can be accomplished by one with ordinary skill without undue experimentation.

In certain embodiments, primer pairs are configured to produce bioagent identifying amplicons within more conserved regions of Picornaviruses while others produce bioagent identifying amplicons within regions that are may evolve more quickly. Primer pairs that characterize amplicons in a conserved region with low probability that the region will evolve past the point of primer recognition are useful, e.g., as a broad range survey-type primer. Primer pairs that characterize an amplicon corresponding to an evolving genomic region are useful, e.g., for distinguishing emerging strain variants.

The primer pairs described herein provide reagents, e.g., for identifying diseases caused by emerging viruses. Base composition analysis eliminates the need for prior knowledge of bioagent sequence to generate hybridization probes. Thus, in another embodiment, there is provided a method for determining the etiology of a virus infection when the process of identification of viruses is carried out in a clinical setting, and even when the virus is a new species. This is possible because the methods may not be confounded by naturally occurring evolutionary variations (a major concern when using probe based or sequencing dependent methods for characterizing viruses that evolve rapidly). Measurement of molecular mass and determination of base composition is accomplished in an unbiased manner without sequence prejudice, and without the need for specificity as is required with probes.

Another embodiment provides a means of tracking the spread of any species or strain of virus when a plurality of samples obtained from different geographical locations are analyzed by methods described above in an epidemiological setting. For example, a plurality of samples from a plurality of different locations may be analyzed with primers which produce bioagent identifying amplicons, a subset of which contains a specific virus. The corresponding locations of the members of the virus-containing subset indicate the spread of the specific virus to the corresponding locations.

Also provided are kits for carrying out the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to fifty primer pairs, from one to twenty primer pairs, from one to ten primer pairs, from one to eight primer pairs or from two to five primer pairs. In some embodiments, the kit may comprise one or more primer pairs recited in Tables 2, 7, 8, and 9.

In some embodiments, the kit may comprise one or more broad range survey primer(s), division wide primer(s), or drill-down primer(s), or any combination thereof A kit may be configured so as to comprise select primer pairs for identification of a particular bioagent. For example, a broad range survey primer kit may be used initially to identify an unknown bioagent as a member of the family Picornaviridae. Another example of a division-wide kit may be used to distinguish human Enterovirus type A from human Enterovirus type B, or from human Enterovirus type C. Another example of a division-wide kit may be used to distinguish human Rhinovirus type A from human Rhinovirus type B. A drill-down kit may be used, for example, to distinguish different serotypes of enteroviruses, rhinoviruses, heptoviruses, cardioviruses or genetically engineered enteroviruses, rhinoviruses, heptoviruses, cardioviruses. In some embodiments, kits may be combined to comprise a combination of broad range survey primers and division-wide primers so as to be able to identify the Picornaviruses. To further illustrate, in certain embodiments, kits include broad Enterovirus/Rhinovirus primer pairs (e.g., primer pairs having primer pair sequences, such as SEQ ID NOS: 2:31, 3:32, 10:39, etc.), Human enterovirus (A-D), Polio Porcine EV primer pairs (e.g., primer pairs having primer pair sequences, such as SEQ ID NOS: 4:33, 5:34, etc.), Rhinovirus primer pairs (e.g., primer pairs having primer pair sequences, such as SEQ ID NOS: 7:36, etc.), broad Cardiovirus primer pairs (e.g., primer pairs having primer pair sequences, such as SEQ ID NOS: 61:73, etc.), and EMCV:3D primer pairs (e.g., primer pairs having primer pair sequences, such as SEQ ID NOS: 64:76, etc.). In some embodiments, the kit may contain standardized calibration polynucleotides for use as internal amplification calibrants.

In some embodiments, the kit may also comprise a sufficient quantity of reverse transcriptase (if an RNA virus, such as members of the Picornaviridae family, is to be identified for example), a DNA polymerase, suitable nucleoside triphosphates (including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. In some embodiments, the kit further comprises instructions for analysis, interpretation and dissemination of data acquired by the kit. In other embodiments, instructions for the operation, analysis, interpretation and dissemination of the data of the kit are provided on computer readable media. A kit may also comprise amplification reaction containers such as microcentrifuge tubes, microtiter plates, and the like. A kit may also comprise reagents or other materials for isolating bioagent nucleic acid or bioagent identifying amplicons from amplification, including, for example, detergents, solvents, or ion exchange resins which may be linked to magnetic beads. A kit may also comprise a table of measured or calculated molecular masses and/or base compositions of bioagents using the primer pairs of the kit.

The invention also provides systems that can be used to perform various assays relating to picornavirus detection or identification. In certain embodiments, systems include mass spectrometers configured to detect molecular masses of amplicons produced using purified oligonucleotide primer pairs described herein. Other detectors that are optionally adapted for use in the systems of the invention are described further below. In some embodiments, systems also include controllers operably connected to mass spectrometers and/or other system components. In some of these embodiments, controllers are configured to correlate the molecular masses of the amplicons with picornavirus bioagents to effect detection or identification (e.g., at genus, species, and/or sub-species levels). In some embodiments, controllers are configured to determine base compositions of the amplicons from the molecular masses of the amplicons. As described herein, the base compositions generally correspond to the picornavirus bioagent identities. In certain embodiments, controllers include or are operably connected to databases of known molecular masses and/or known base compositions of amplicons of known picornavirus bioagents produced with the primer pairs described herein. Controllers are described further below.

In some embodiments, systems include one or more of the primer pairs described herein (e.g., in Tables 2, 7, 8, and 9). In certain embodiments, the oligonucleotides are arrayed on solid supports, whereas in others, they are provided in one or more containers, e.g., for assays performed in solution. In certain embodiments, the systems also include at least one detector or detection component (e.g., a spectrometer) that is configured to detect detectable signals produced in the container or on the support. In addition, the systems also optionally include at least one thermal modulator (e.g., a thermal cycling device) operably connected to the containers or solid supports to modulate temperature in the containers or on the solid supports, and/or at least one fluid transfer component (e.g., an automated pipettor) that transfers fluid to and/or from the containers or solid supports, e.g., for performing one or more assays (e.g., nucleic acid amplification, real-time amplicon detection, etc.) in the containers or on the solid supports.

Detectors are typically structured to detect detectable signals produced, e.g., in or proximal to another component of the given assay system (e.g., in a container and/or on a solid support). Suitable signal detectors that are optionally utilized, or adapted for use, herein detect, e.g., fluorescence, phosphorescence, radioactivity, absorbance, refractive index, luminescence, or mass. Detectors optionally monitor one or a plurality of signals from upstream and/or downstream of the performance of, e.g., a given assay step. For example, detectors optionally monitor a plurality of optical signals, which correspond in position to “real-time” results. Example detectors or sensors include photomultiplier tubes, CCD arrays, optical sensors, temperature sensors, pressure sensors, pH sensors, conductivity sensors, or scanning detectors. Detectors are also described in, e.g., Skoog et al., Principles of Instrumental Analysis, 5^{th} Ed., Harcourt Brace College Publishers (1998), Currell, Analytical Instrumentation: Performance Characteristics and Quality, John Wiley & Sons, Inc. (2000), Sharma et al., Introduction to Fluorescence Spectroscopy, John Wiley & Sons, Inc. (1999), Valeur, Molecular Fluorescence: Principles and Applications, John Wiley & Sons, Inc. (2002), and Gore, Spectrophotometry and Spectrofluorimetry: A Practical Approach, 2.sup.nd Ed., Oxford University Press (2000), which are each incorporated by reference.

As mentioned above, the systems of the invention also typically include controllers that are operably connected to one or more components (e.g., detectors, databases, thermal modulators, fluid transfer components, robotic material handling devices, and the like) of the given system to control operation of the components. More specifically, controllers are generally included either as separate or integral system components that are utilized, e.g., to receive data from detectors (e.g., molecular masses, etc.), to effect and/or regulate temperature in the containers, to effect and/or regulate fluid flow to or from selected containers. Controllers and/or other system components are optionally coupled to an appropriately programmed processor, computer, digital device, information appliance, or other logic device (e.g., including an analog to digital or digital to analog converter as needed), which functions to instruct the operation of these instruments in accordance with preprogrammed or user input instructions, receive data and information from these instruments, and interpret, manipulate and report this information to the user. Suitable controllers are generally known in the art and are available from various commercial sources.

Any controller or computer optionally includes a monitor, which is often a cathode ray tube (“CRT”) display, a flat panel display (e.g., active matrix liquid crystal display or liquid crystal display), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. These components are illustrated further below.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming.

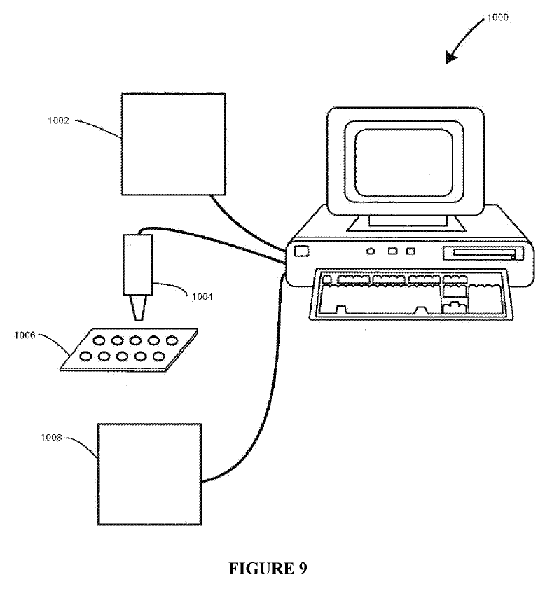

FIG. 9 is a schematic showing a representative system that includes a logic device in which various aspects of the present invention may be embodied. As will be understood by practitioners in the art from the teachings provided herein, aspects of the invention are optionally implemented in hardware and/or software. In some embodiments, different aspects of the invention are implemented in either client-side logic or server-side logic. As will be understood in the art, the invention or components thereof may be embodied in a media program component (e.g., a fixed media component) containing logic instructions and/or data that, when loaded into an appropriately configured computing device, cause that device to perform as desired. As will also be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

More specifically, FIG. 9 schematically illustrates computer 1000 to which mass spectrometer 1002 (e.g., an ESI-TOF mass spectrometer, etc.), fluid transfer component 1004 (e.g., an automated mass spectrometer sample injection needle or the like), and database 1008 are operably connected. Optionally, one or more of these components are operably connected to computer 1000 via a server (not shown in FIG. 9). During operation, fluid transfer component 1004 typically transfers reaction mixtures or components thereof (e.g., aliquots comprising amplicons) from multi-well container 1006 to mass spectrometer 1002. Mass spectrometer 1002 then detects molecular masses of the amplicons. Computer 1000 then typically receives this molecular mass data, calculates base compositions from this data, and compares it with entries in database 1008 to effect identification of picornavirus bioagents in a given sample. It will be apparent to one of skill in the art that one or more components of the system schematically depicted in FIG. 9 are optionally fabricated integral with one another (e.g., in the same housing).

While the present invention has been described with specificity in accordance with certain of its embodiments, the following examples serve only to illustrate the invention and are not intended to limit the same. In order that the invention disclosed herein may be more efficiently understood, examples are provided below. It should be understood that these examples are for illustrative purposes only and are not to be construed as limiting the invention in any manner.

### EXAMPLE 1

### Slection and Validation of Primers that Define Bioagent Identifying Amplicons for Picornaviruses

For design of primers that define Picornaviridae family member identifying amplicons, a series of Enterovirus and Rhinovirus genome segment sequences were obtained, aligned and scanned for regions where pairs of PCR primers would amplify products of about 45 to about 150 nucleotides in length and distinguish species and/or individual strains from each other by their molecular masses or base compositions. A typical process shown in FIG. 1 is employed for this type of analysis.

A database of expected base compositions for each primer region was generated using an in silico PCR search algorithm (i.e., ePCR). An existing RNA structure search algorithm (Macke et al., Nucl. Acids Res., 2001, 29, 4724-4735, which is incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This also provides information on primer specificity of the selected primer pairs.

In addition to the broad range Enterovirus/Rhinovirus primers, several other primers specific to 5′ UTR of the broad Enterovirus genome, 5′ UTR human Enterovirus, 5′ UTR of the poliovirus, 5′ UTR of the Porcine Enterovirus, 5′ UTR of Rhinovirus, UTR of bovine Enterovirus, 3D gene of poliovirus, 3C gene of poliovirus, 2C gene of Coxsackie virus A, 3D gene of Coxsackie virus A, 3D gene of Coxsackie virus B, 2C gene of Coxsackie virus B, 3C gene of Coxsackie virus B, 3D gene of Coxsackie virus C, 3D gene of Human Rhinovirus A, 2C gene of Human Rhinovirus A, VP1 gene of Human Rhinovirus B, and 2B gene of Human Rhinovirus B were designed to detect and identify naturally occurring members of the Enterovirus and Rhinovirus genus as well as several artificial chimera constructs consisting of regions from two different viruses. Table 1 shows the primer pair identifiers and the Enterovirus and Rhinovirus gene segments/regions that were used for primer design and the specificity of the target viral species.

TABLE 1
Primer Pair Name Identifiers for Selected Viruses
And Numbers of Primer Pairs Targeting 5′ UTR and Genes for
Amplification of Enterovirus Species

| | Primer Pair Name | | Number of |
|---|---|---|---|
| Virus | Virus Identifier | Gene/Region | Primer Pairs |
| Broad Enterovirus/ | GENOME5UTR | 5′ UTR | 2 |
| Rhinovirus | | | |
| Human Enterovirus | GENOME5UTR | 5′ UTR | 3 |
| (A-D), Polio. | | | |
| Porcine EV | | | |
| Rhinovirus | GENOME5UTR | 5′ UTR | 1 |
| Bovine Enterovirus | GENOMEUTR | 5′ UTR | 2 |
| Poliovirus | POLIO5UTR | 5′ UTR | 2 |
| Poliovirus | POLIO3D | 3D | 3 |
| Poliovirus | POLIO3C | 3C | 2 |
| Coxsackie Virus A | COXA3D | 3D | 1 |
| Coxsackie Virus A | COXA2C | 2C | 1 |
| Coxsackie Virus B | COXB3D | 3D | 2 |
| Coxsackie Virus B | COXB2C | 2C | 1 |
| Coxsackie Virus B | COXB3C | 3C | 1 |
| Coxsackie Virus C | COXC3D | 3D | 4 |
| Human Rhinovirus A | HRVA3D | 3D | 1 |
| Human Rhinovirus A | HRVA2C | 2C | 1 |
| Human Rhinovirus B | HRVBVP1 | VP1 | 1 |
| Human Rhinovirus B | HRVB2B | 2B | 1 |
| | | | Total: 29 |

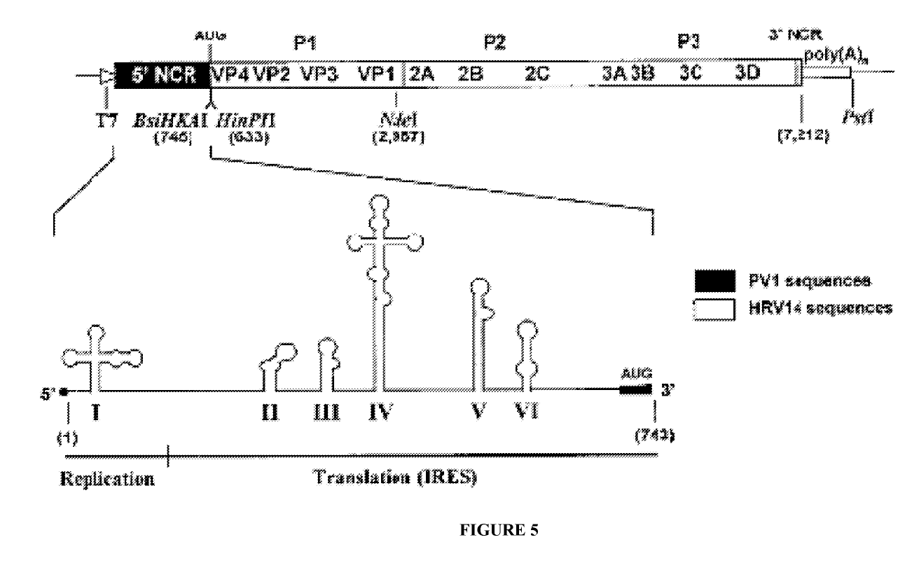

A total of 29 primer pairs were designed, of which four were targeted broadly to all known Enterovirus/Rhinovirus species (primer names containing “GENOME5UTR”). The remaining were species type-specific as shown in Table 2, which is a collection of primers (sorted by forward primer name) designed to identify enterovirus and rhinovirus serotypes using the methods described herein. Both a calibrant construct and a positive control construct were also designed and ordered from Blue Heron Biotechnology, Bothell, Wash. These are artificial constructs containing sequences that are amplified by the primer pairs of interest, but cannot be confused with viral sequences by base composition. The primer pair number is an in-house database index number. Primer sites were identified on Enterovirus genes or related nucleic acid sequences including 5′UTR, Polio3D, Polio3C, COXA2C, COXA3D, COXB3D, COXB2C, COXB3C, and COXC3D. Primer sites were identified on Rhinovirus genes including HRVA3D, HRVA2C, HRVBVP1, and HRVB2B. The following viral samples were used as well in configuring the primer pairs to detect and identify naturally occurring members of the Enterovirus and Rhinovirus genuses as well as artificially constructed chimera consisting of regions from two different viruses: WTPV1, a wild type poliovirus derived from a full length poliovirus laboratory construct, WTHRV14, a wild type human rhinovirus type 14, WTCVB3, a wild type coxsackievirus type B3, PCV305, a poliovirus/coxsackievirus chimera that was derived from an artificial construct consisting of a coxsackievirus 5′ end insertion in a poliovirus genome, PV5′HRV14, a poliovirus/human rhinovirus 14 chimera that was derived from an artificial construct consisting of a poliovirus 5′ end insertion in a human rhinovirus 14 genome (see, FIG. 5). It should be noted that various primer designs failed to produce the desired levels of resolution within the Picornaviridae family. For example, primer pair 3763, which was designed to target Rhinoviruses, failed to do so in all tests.

The forward or reverse primer name shown in Table 2 indicates the gene region of the viral genome to which the primer hybridizes relative to a reference sequence. The forward primer name GENMOE5UTR_NC001472-1-7389_{—}445_{—}463_F indicates that the forward primer (“_F”) hybridizes to residues 445-463 (“445_{—}463”) of the 5′ UTR region of the genome (“5UTR”) of a reference virus. In this example the reference virus is human Enterovirus B genome sequence (“GENOME”) containing bases 1-7389 (“-1-7389_”) referenced in GenBank as accession number NC_{—}001472 (“_NC001472_“) (SEQ ID NO: 1). The reference virus nomenclature in the primer name is selected to provide a reference, and does not necessarily mean that the primer pair has been designed with 100% complementarity to that target site on the reference virus. A description of the primer design is provided herein.

TABLE 2
Primer Pairs for Identification of Enteroviruses and Rhinoviruses

| Primer | Primer | Primer | Forward | | Forward | Forward | Reverse | | Reverse | Reverse |
|---|---|---|---|---|---|---|---|---|---|---|
| pair | pair | pair | primer | Forward | primer | SEQ ID | primer | Reverse | primer | SEQ ID |
| number | code | name | code | primer name | sequence | NO: | code | primer name | sequence | NO: |
| 3758 | VIR3758 | GENOM | VIR8676F | GENOME5UTR_{—} | TTCCTCC | 2 | VIR8677R | GENOME5UTR_{—} | TGAAACA | 31 |
| | | E5UTR_{—} | | NC001472-1- | GGCCCCT | | | NC001472-1- | CGGGCAC | |
| | | NC0014 | | 7389_445_463_F | GAATG | | | 7389_543_56 | CGAAAGT | |
| | | 72-1- | | | | | | 6_R | AGT | |
| | | 7389_4 | | | | | | | | |
| | | 45_566 | | | | | | | | |
| 3759 | VIR3759 | GENOM | VIR8678F | GENOME5UTR_{—} | TCCGGCC | 3 | VIR8679R | GENOME5UTR_{—} | TGAAACA | 32 |
| | | E5UTR_{—} | | NC001472-1- | CCTGAAT | | | NC001472-1- | CGGACAC | |
| | | NC0014 | | 7389_449_469_F | GCGGCTA | | | 7389_540_56 | CCAAAGT | |
| | | 72-1- | | | | | | 6_R | AGTCGG | |
| | | 7389_4 | | | | | | | | |
| | | 49_566 | | | | | | | | |
| 3760 | VIR3760 | GENOM | VIR8680F | GENOME5UTR_{—} | TGGCTGC | 4 | VIR8681R | GENOME5UTR_{—} | TAGCCGC | 33 |
| | | E5UTR_{—} | | NC001472-1- | GTTGGCG | | | NC001472-1- | ATTCAGG | |
| | | NC0014 | | 7389_358_374_F | GCC | | | 7389_449_46 | GGCCGGA | |
| | | 72-1- | | | | | | 9_R | | |
| | | 7389_3 | | | | | | | | |
| | | 58_469 | | | | | | | | |
| 3761 | VIR3761 | GENOM | VIR8682F | GENOME5UTR_{—} | TCTACTT | 5 | VIR8683R | GENOME5UTR_{—} | TCATTGT | 34 |
| | | E5UTR_{—} | | NC001472-1- | TGGGTGT | | | NC001472-1- | CACCATA | |
| | | NC0014 | | 7389_543_565_F | CCGTGTT | | | 7389_581_60 | AGCAGCC | |
| | | 72-1- | | | TC | | | 2_R | A | |
| | | 7389_5 | | | | | | | | |
| | | 43_602 | | | | | | | | |
| 3762 | VIR3762 | GENOM | VIR8684F | GENOME5UTR_{—} | TCAGCCT | 6 | VIR8685R | TAAAACA | GGCGCAC | 35 |
| | | E5UTR_{—} | | NC001472-1- | GTGGGTT | | | GENOME5UTR_{—} | AAGGGTA | |
| | | NC0014 | | 7389_6_27_F | GTACCCA | | | NC001472-1- | CC | |
| | | 72-1- | | | C | | | 7389_64_86_R | | |
| | | 738_9_{—} | | | | | | | | |
| | | 686 | | | | | | | | |
| 3763 | VIR3763 | GENOM | VIR8686F | GENOME5UTR_{—} | TTCTAGC | 7 | VIR8687R | GENOME5UTR_{—} | TAGCACA | 36 |
| | | E5UTR_ | | NC001490-1- | CTGCGTG | | | NC001490-1- | CGCGGGT | |
| | | NC0014 | | 7212_365_384_F | GCTGCC | | | 7212_421_44 | CTTCACA | |
| | | 90-1- | | | | | | 3_R | CC | |
| | | 7212_3 | | | | | | | | |
| | | 65_443 | | | | | | | | |
| 3764 | VIR3764 | GENOM | VIR8688F | GENOMEUTR_N | TCCTCCG | 8 | VIR8689R | GENOMEUTR_{—} | TGAAACA | 37 |
| | | EUTR_N | | C001859-1- | CGCCGTG | | | NC001859-1- | CGGAGTC | |
| | | C00185 | | 7414_524_543_F | GAATGC | | | 7414_620_64 | CCGAAAG | |
| | | 9-1- | | | | | | 5_R | TAGTC | |
| | | 7414_5 | | | | | | | | |
| | | 24_645 | | | | | | | | |
| 3765 | VIR3765 | GENOM | VIR8690F | GENOMEUTR_N | TCCCACC | 9 | VIR8691R | GENOMEUTR_{—} | TGGGAAA | 38 |
| | | EUTR_N | | C001859-1- | ATGGGGC | | | NC001859-1- | ACAGGCG | |
| | | C00185 | | 7414_22_40_F | C | | | 7414_68_90_R | TACAAAG | |
| | | 9-1- | CCAC | | | | | | GT | |
| | | 7414_2 | | | | | | | | |
| | | 2_90 | | | | | | | | |
| 3835 | VIR3835 | POLIOS | VIR8814F | POLIO5UTR_NC | TCCTCCG | 10 | VIR8815R | POLIO5UTR_NC | TGAAACA | 39 |
| | | UTR_N | | 002058-1- | GCCCCTG | | | 002058-1- | CGGACAC | |
| | | C00205 | | 7440_443_460_F | AATG | | | 7440_541_56 | CCAAAGT | |
| | | 8-1- | | | | | | 3_R | AG | |
| | | 7440_4 | | | | | | | | |
| | | 43_563 | | | | | | | | |
| 3836 | VIR3836 | POLIO5 | VIR8816F | POLIO5UTR_NC | TGGTACC | 11 | VIR8817R | POLIO5UTR_NC | TCCGGGG | 40 |
| | | UTR_N | | 002058-1- | TTTGTAC | | | 002058-1- | AAACAGA | |
| | | C00205 | | 7440_65_85_F | GCCTGTT | | | 7440_162_18 | AGTGCTT | |
| | | 8-1- | | | | | | 3_R | G | |
| | | 7440_6 | | | | | | | | |
| | | 5_183 | | | | | | | | |
| 3837 | VIR3837 | POLIO3 | VIR8818F | POLIO3D_NC00 | TATGGTG | 12 | VIR8819R | POLIO3D_NC00 | TAGTCTT | 41 |
| | | D_NC00 | | 2058-1- | ATGATGT | | | 2058-1- | TTCCTGA | |
| | | 2058-1- | | 7440_6962_699 | AATTGCT | | | 7440_7007_70 | TTGGGCT | |
| | | 7440_6 | | 3_F | TCCTACC | | | 35_R | AGGAGAC | |
| | | 962_70 | | | CCCA | | | | T | |
| | | 35 | | | | | | | | |
| 3838 | VIR3838 | POLIO3 | VIR8820F | POLIO3D_NC00 | TACTCAA | 13 | VIR8821R | POLIO3D_NC00 | TAACCCA | 42 |
| | | D_NC00 | | 2058-1- | CATTGTA | | | 2058-1- | ATCCAAT | |
| | | 2058-1- | | 7440_7334_735 | CCGCCGT | | | 7440_7376_74 | TCGACTG | |
| | | 7440_7 | | 9_F | TGGCT | | | 04_R | AGGTAGG | |
| | | 334_74 | | | | | | | G | |
| | | 04 | | | | | | | | |
| 3839 | VIR3839 | POLIO3 | VIR8822F | PO LIO3D_NC00 | TAAGGGC | 14 | VIR8823R | POLIO3D_NC00 | TAGGTGG | 43 |
| | | D_NC00 | | 2058-1- | GGCATGC | | | 2058-1- | TCTAAAT | |
| | | 2058-1- | | 7440_6832_685 | CATCTGG | | | 7440_6920_69 | CTATGCC | |
| | | 7440_6 | | 2_F | | | | 49_R | CTTGTAG | |
| | | 832_69 | | | | | | | GT | |
| | | 49 | | | | | | | | |
| 3840 | VIR3840 | POLIO3 | VIR8824F | POLIO3C_NC00 | TGCATGT | 15 | VIR8825R | POLI03C_NC00 | TGAGTGA | 44 |
| | | C__C00 | | 2058-1- | TGGTGGG | | | 2058-1- | AGTATGA | |
| | | 2058-1- | | 7440_5916_593 | AACGGTT | | | 7440_5954_59 | TCGCTTT | |
| | | 7440_5 | | 7_F | C | | | 79_R | AGGGC | |
| | | 916_59 | | | | | | | | |
| | | 79 | | | | | | | | |
| 3841 | VIR3841 | POLIO3 | VIR8826F | POLIO3C_NC00 | TCAAATC | 16 | VIR8827R | POLI03C_NC00 | TGTTGGG | 45 |
| | | C_NC00 | | 2058-1- | ACTGAGA | | | 2058-1- | GTACTTG | |
| | | 2058-1- | | 7440_5713_573 | CAAATGA | | | 7440_5747_57 | CTAGTGT | |
| | | 7440_5 | | 9_F | TGGAGT | | | 71_R | TCAC | |
| | | 713_57 | | | | | | | | |
| | | 71 | | | | | | | | |
| 3842 | VIR3842 | COXA3 | VIR8828F | COXA3D_U0587 | TGAACCC | 17 | VIR8829R | COXA3D_U058 | TATTCTG | 46 |
| | | D_U058 | | 6-1- | CACCAGA | | | 76-1- | GTTATAA | |
| | | 76-1- | | 7413_7347_737 | AATCTGG | | | 7413_7380_74 | CAAATTC | |
| | | 7413_7 | | 2_F | TCGTG | | | 11_R | ACCCCCA | |
| | | 347_74 | | | | | | | CCAG | |
| | | 11 | | | | | | | | |
| 3843 | VIR3843 | COXA2C_{—} | VIR8830F | COXA2C_U0587 | TGAACAA | 18 | VIR8831R | COXA2C_U0587 | TCAGACA | 47 |
| | | U0587 | | 6-1- | CTACATG | | | 6-1- | TACAGGT | |
| | | 6-1- | | 7413_4406_443 | CAGTTCA | | | 7413_4435_44 | TCAATAC | |
| | | 7413_4 | | 1_F | AGAGC | | | 59_R | GGTG | |
| | | 406_44 | | | | | | | | |
| | | 59 | | | | | | | | |
| 3844 | VIR3844 | COXB3 | VIR8832F | COXB3D_M165 | TAACCCT | 19 | VIR8833R | COXB3D_M165 | TCACCGA | 48 |
| | | D_M16 | | 60-1- | ACTGCGC | | | 60-1- | ATGCGGA | |
| | | 560-1- | | 7389_7324_734 | TAACCGA | | | 7389_7365_73 | GAATTTA | |
| | | 7389_7 | | 6_F | AC | | | 88_R | CCC | |
| | | 324_73 | | | | | | | | |
| | | 88 | | | | | | | | |
| 3845 | VIR3845 | COXB2C_{—} | VIR8834F | COXB2C_M165 | TGAGCAA | 20 | VIR8835R | COXB2C_M165 | TAAGCAT | 49 |
| | | M165 | | 60-1- | TTACATA | | | 60-1- | ACAGGTT | |
| | | 60-1- | | 7389_4355_438 | ACGTTCA | | | 7389_4384_44 | CAATACG | |
| | | 7389_4 | | 1_F | AGTCCA | | | 07_R | GCA | |
| | | 355_44 | | | | | | | | |
| | | 07 | | | | | | | | |
| 3846 | VIR3846 | COXB3 | VIR8836F | COXB3D_M165 | TAGGATG | 21 | VIR8837R | COXB3D_M165 | TCTCCTT | 50 |
| | | D_M16 | | 60-1- | ATCGCTT | | | 60-1- | GTCTGCT | |
| | | 560-1- | | 7389_6867_689 | ATGGTGA | | | 7389_6964_69 | TGGTGTC | |
| | | 7389_6 | | 6_F | GTATGTG | | | 86_R | AT | |
| | | 867_69 | | | A | T | | | | |
| | | 86 | | | | | | | | |
| 3847 | VIR3847 | COXB3C_{—} | VIR8838F | COXB3C_M165 | TGGCATC | 22 | VIR8839R | COXB3C_M165 | TCCAGGT | 51 |
| | | M165 | | 60-1- | ATTGACA | | | 60-1- | TTGGCAT | |
| | | 60-1- | | 7389_5436_545 | GGTGGGC | | | 7389_5464_54 | GGCGTGG | |
| | | 7389_5 | | 6_F | | | | 84_R | | |
| | | 436_54 | | | | | | | | |
| | | 84 | | | | | | | | |
| 3848 | VIR3848 | COXC3 | VIR8840F | COXC3D_D0053 | TAGTAAC | 23 | VIR8841R | COXC3D_D0053 | TCCGAAT | 52 |
| | | D_D005 | | 8-1- | CCTACCT | | | 8-1- | TAAAGGA | |
| | | 38-1- | | 7401_7330_735 | CAGCCGA | | | 7401_7372_74 | AAATTTA | |
| | | 7401_7 | | 3_F | ATT | | | 00_R | CCCCTAC | |
| | | 330_74 | | | | | | | A | |
| | | 00 | | | | | | | | |
| 3849 | VIR3849 | COXC3 | VIR8842F | COXC3D_D0053 | TCTCCTA | 24 | VIR8843R | COXC3D_D0053 | TCTGCTC | 53 |
| | | D_D005 | | 8-1- | GCCCAAT | | | 8-1- | TGAAGAA | |
| | | 38-1- | | 7401_6969_699 | CAGGAAA | | | 7401_7059_70 | TCTTTTC | |
| | | 7401_6 | | 5_F | AGACTA | | | 88_R | AGGAATG | |
| | | 969_70 | | | | | | | TT | |
| | | 88 | | | | | | | | |
| 3850 | VIR3850 | COXC3 | VIR8844F | COXC3D_D0053 | TGCCAAT | 25 | VIR8845R | COXC3D_D0053 | TCCGTTG | 54 |
| | | D_D005 | | 8-1- | GAAAGAA | | | 8-1- | TGCCAAG | |
| | | 38-1- | | 7401_7121_715 | ATTTATG | | | 7401_7198_72 | CCAATAG | |
| | | 7401_7 | | 7_F | AATCAAT | | | 21_R | GCA | |
| | | 121_72 | | | TAGATGG | | | | | |
| | | 21 | | | AC | | | | | |
| 3851 | VIR3851 | COXC3 | VIR8846F | COXC3D_D0053 | TAACATT | 26 | VIR8847R | COXC3D_D0053 | TGATTCA | 55 |
| | | D_D005 | | 8-1- | CCTGAAA | | | 8-1- | TGAATTT | |
| | | 38-1- | | 7401_7058_709 | AGATTCT | | | 7401_7114_71 | CTTTCAT | |
| | | 7401_7 | | 1_F | TCAGAGC | | | 46_R | TGGCATT | |
| | | 058_71 | | | AGATGA | | | | ACTGG | |
| | | 46 | | | | | | | | |
| 3852 | VIR3852 | HRVA3 | VIR8848F | HRVA3D_NC_0 | TGTGTCA | 27 | VIR8849R | HRVA3D_NC_0 | TGGGATA | 56 |
| | | D_NC_0 | | 01617-1- | CTTAATG | | | 01617-1- | TACAGTG | |
| | | 01617- | | 7152_6977_700 | TGGCACA | | | 7152_7048_70 | CGCGACC | |
| | | 1- | | 1_F | GATG | | | 71_R | AGC | |
| | | 7152_6 | | | | | | | | |
| | | 977_70 | | | | | | | | |
| | | 71 | | | | | | | | |
| 3853 | VIR3853 | HRVA2 | VIR8850F | HRVA2C_NC001 | TATTTTG | 28 | VIR8851R | HRVA2C_NC00 | TGGATTT | 57 |
| | | C_NC00 | | 617-1- | ATGGTTA | | | 1617-1- | TGCATGA | |
| | | 1617-1- | | 7152_4393_442 | TGATCAG | | | 7152_4429_44 | TGTCATC | |
| | | 7152_4 | | 1_F | CAGAGTG | | | 52_R | CAT | |
| | | 393_44 | | | T | | | | | |
| | | 52 | | | | | | | | |
| 3854 | VIR3854 | HRVBV | VIR8852F | HRVBVP1_NC00 | TGATTAC | 29 | VIR8853R | HRVBVP1_NC0 | TCATCAT | 58 |
| | | P1_NC0 | | 1490-1- | ACATGGC | | | 01490-1- | GTGAGTA | |
| | | 01490- | | 7212_2821_284 | AGAGTGC | | | 7212_2924_29 | ACCATCA | |
| | | 1- | | 1_F | | | | 52_R | TAGAAAC | |
| | | 7212_2 | | | | | | | A | |
| | | 821_29 | | | | | | | | |
| | | 52 | | | | | | | | |
| 3855 | VIR3855 | HRVB2B | VIR8854F | HRVB2B_NC001 | TGGATGT | 30 | VIR8855R | HRVB2B_NC00 | TGAACCA | 59 |
| | | NC001 | | 490-1- | GATGGTT | | | 1490-1- | GCCATCA | |
| | | 490-1- | | 7212_3847_387 | CTCCATG | | | 7212_3917_39 | TTTGCTT | |
| | | 7212_3 | | 0_F | GAG | | | 44_R | GCCTTTC | |
| | | 847_39 | | | | | | | | |
| | | 44 | | | | | | | | |

These primers were tested against a panel of Enterovirus and Rhinovirus viral calibrants. The test panel included Enterovirus and Rhinovirus calibrants and positive control constructs (IPC) as shown in Table 3.

TABLE 3
Strains of Enterovirus/Rhinovirus Species used for
Testing Primer of Table 2 and their base compositions

| | | | | | | | | OPPOSITE_{—} |
|---|---|---|---|---|---|---|---|---|
| Primer | | | | | MONO_{—} | | OPPOSITE_{—} | STRAND_{—} |
| pair | RECORD_{—} | | | | EXACT_{—} | BASE_{—} | STRAND_{—} | BASE_{—} |
| Number | ORIGIN | GI | ORGANISM | STRAIN | MASS | COMP | MASS | COMP |
| 3758 | CALIBRA | Isis\|C | CALIBRAN | ENTER | 35843 | A24 | 36300. | A26 |
| | NTS | AL000 | T_ENTERO | OVCAL | | G28 | 9935 | G39 |
| | | 134 | VCAL | | | C39 | | C28 |
| | | | | | | T26 | | T24 |
| 3759 | CALIBRA | Isis\|C | CALIBRAN | ENTER | 34712 | A24 | 34960. | A25 |
| | NTS | AL000 | T_ENTERO | OVCAL | | G29 | 7722 | G35 |
| | | 134 | VCAL | | | C35 | | C29 |
| | | | | | | T25 | | T24 |
| 3760 | CALIBRA | Isis\|C | CALIBRAN | ENTER | 33115 | A21 | 32853. | A23 |
| | NTS | AL000 | T_ENTERO | OVCAL | | G35 | 4295 | G28 |
| | | 134 | VCAL | | | C28 | | C35 |
| | | | | | | T23 | | T21 |
| 3761 | CALIBRA | Isis\|C | CALIBRAN | ENTER | 16902 | A9 | 16938. | A22 |
| | NTS | AL000 | T_ENTERO | OVCAL | | G13 | 9064 | G11 |
| | | 134 | VCAL | | | C11 | | C13 T9 |
| | | | | | | T22 | | |
| 3762 | CALIBRA | Isis\|C | CALIBRAN | ENTER | 23229 | Al2 | 23589. | A21 |
| | NTS | AL000 | T_ENTERO | OVCAL | | G18 | 9541 | G25 |
| | | 134 | VCAL | | | C25 | | C18 |
| | | | | | | T21 | | T12 |
| 3763 | CALIBRA | Isis\|C | CALIBRAN | RHINO | 22749 | A14 | 22833. | A19 |
| | NTS | AL000 | T_RHINOV | VICAL | | G20 | 8136 | G21 |
| | | 135 | ICAL | | | C21 | | C20 |
| | | | | | | T19 | | T14 |
| 3764 | CALIBRA | Isis\|C | CALIBRAN | RHINO | 35948 | A24 | 36196. | A25 |
| | NTS | AL000 | T_RHINOV | VICAL | | G31 | 97 | G37 |
| | | 135 | ICAL | | | C37 | | C31 |
| | | | | | | T25 | | T24 |
| 3765 | CALIBRA | Isis\|C | CALIBRAN | RHINO | 19514 | A8 | 19893. | A19 |
| | NTS | AL000 | T_RHINOV | VICAL | | G15 | 358 | G22 |
| | | 135 | ICAL | | | C22 | | C15 T8 |
| | | | | | | T19 | | |
| 3758 | IPC | Isis\|IP | IPC_ENTE | ENTER | 34279 | A23 | 34776. | A25 |
| | | C0000 | ROVIPC | OVIPC | | G26 | 7446 | G38 |
| | | 60 | | | | C38 | | C26 |
| | | | | | | | T25 | T23 |
| 3759 | IPC | Isis\|IP | IPC_ENTE | ENTER | 33147 | A23 | 33436. | A24 |
| | | C0000 | ROVIPC | OVIPC | | G27 | 5233 | G34 |
| | | 60 | | | | C34 | | C27 |
| | | | | | | T24 | | T23 |
| 3760 | IPC | Isis\|IP | IPC_ENTE | ENTER | 31558 | A18 | 31323. | A23 |
| | | C0000 | ROVIPC | OVIPC | | G34 | 1925 | G27 |
| | | 60 | | | | C27 | | C34 |
| | | | | | | T23 | | T18 |
| 3761 | IPC | Isis\|IP | IPC_ENTE | ENTER | 15403 | A8 | 15350. | A20 G9 |
| | | C0000 | ROVIPC | OVIPC | | G13 | 6401 | C13 T8 |
| | | 60 | | | | C9 T20 | | |
| 3762 | IPC | Isis\|IP | IPC_ENTE | ENTER | 21665 | A11 | 22065. | A20 |
| | | C0000 | ROVIPC | OVIPC | | G16 | 7052 | G24 |
| | | 60 | | | | C24 | | C16 |
| | | | | | | T20 | | T11 |
| 3763 | IPC | Isis\|IP | IPC_RHIN | RHINO | 21199 | A13 | 21293. | A19 |
| | | C0000 | OVIIPC | VII PC | | G18 | 5698 | G19 |
| | | 61 | | | | C19 | | C18 |
| | | | | | | T19 | | T13 |
| 3764 | IPC | Isis\|IP | IPC_RHIN | RHINO | 34424 | A23 | 34632. | A24 |
| | | C0000 | OVIIPC | VII PC | | G30 | 715 | G35 |
| | | 61 | | | | C35 | | C30 |
| | | | | | | T24 | | T23 |
| 3765 | IPC | Isis\|IP | IPC_RHIN | RHINO | 17959 | A8 | 18360. | A17 |
| | | C0000 | OVIIPC | VII PC | | G13 | 0976 | G21 |
| | | 61 | | | | C21 | | C13 T8 |
| | | | | | | T17 | | |

Picornavirus target genes, specifically Enterovirus and Rhinovirus target genes are shown along with their observed base composition signatures for a diverse panel of viral isolates.

### Validation of Primers Designed for Identification of Enterovirus

A dilution to extinction (DTE) analysis was performed to demonstrate the sensitivity of the primer pairs for detection. The results are shown in Table 4. An “X” indicates that the target was detected with the indicated primer pair and the indicated copy number.

TABLE 4
Validation of Enterovirus Primer Pairs, DTE of Enterovirus
primer pairs against Calibrant construct RNA

| | | Calibrant Copies/rxn | | | | | | |
|---|---|---|---|---|---|---|---|---|
| Calibrant* | PP | 0 | 1 | 14 | 144 | 1439 | 14390 | 143897 |
| ENTEROVCAL | VIR3758 | | | | | x | X | x |
| | VIR3759 | | | x | x | x | X | x |
| | VIR3760 | | | | x | x | X | x |
| | VIR3761 | | | x | x | x | X | x |
| RHINOVICAL | VIR3763 | | | x | x | x | X | x |
| | VIR3764 | | | x | x | x | X | x |
| | VIR3765 | | | | x | x | X | x |

As shown in Table 4, primer pairs were able to detect the synthetic DNA calibrant for Enterovirus and Rhinovirus at a concentration of at least 1 calibrant copies per reaction, while several could detect more dilute concentrations.

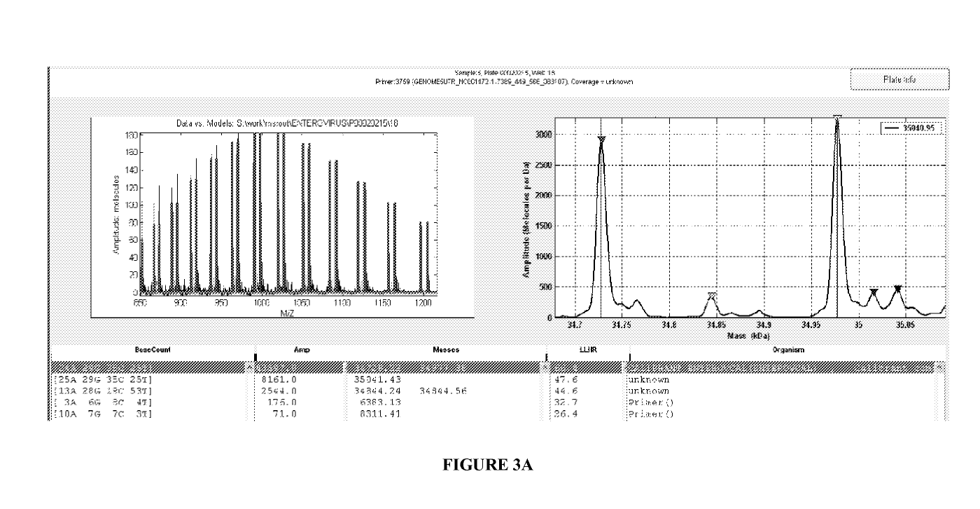

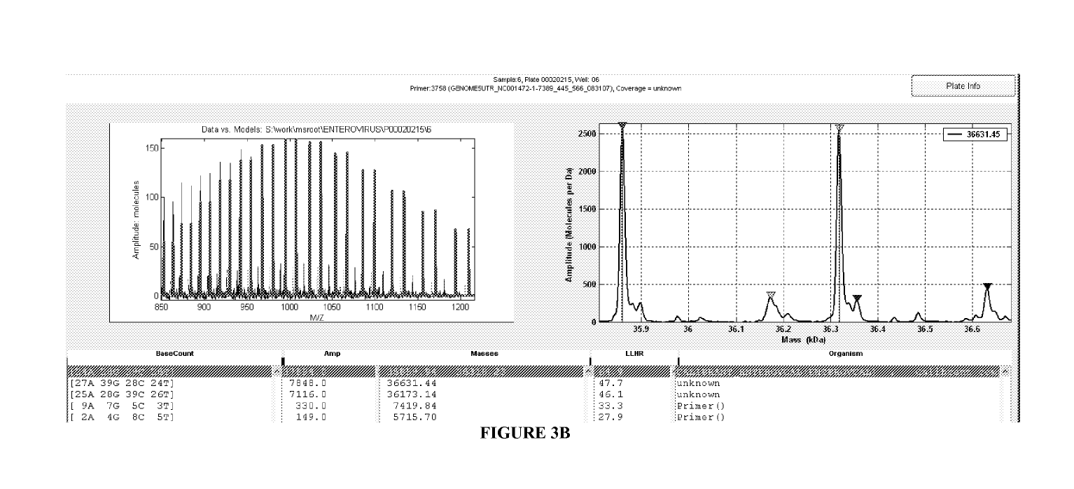

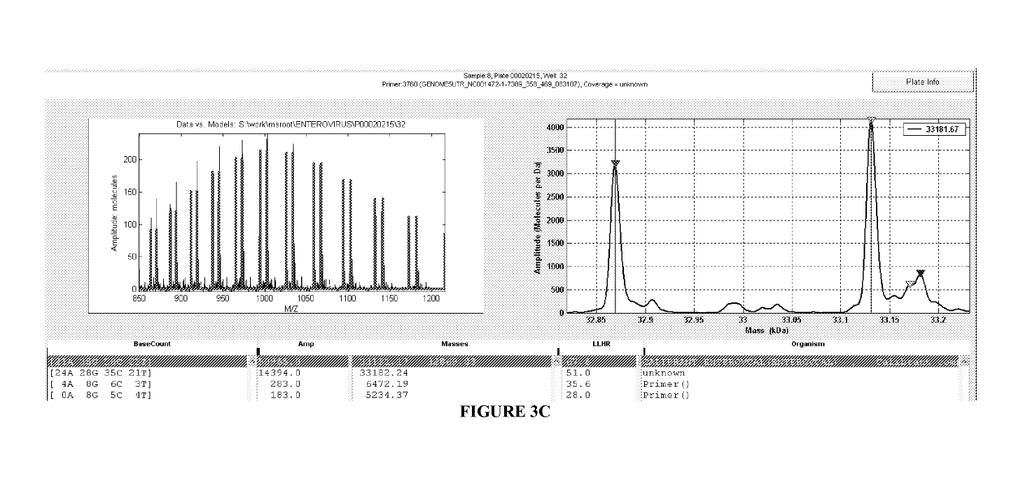

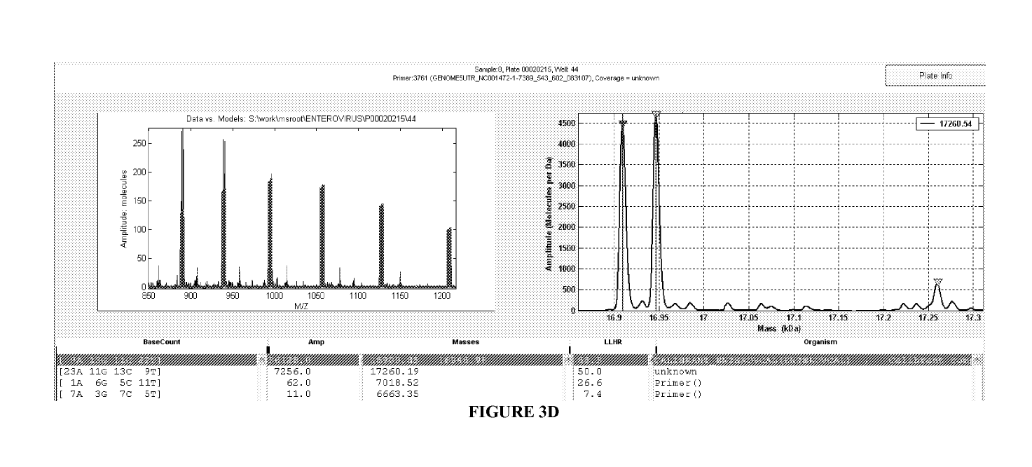

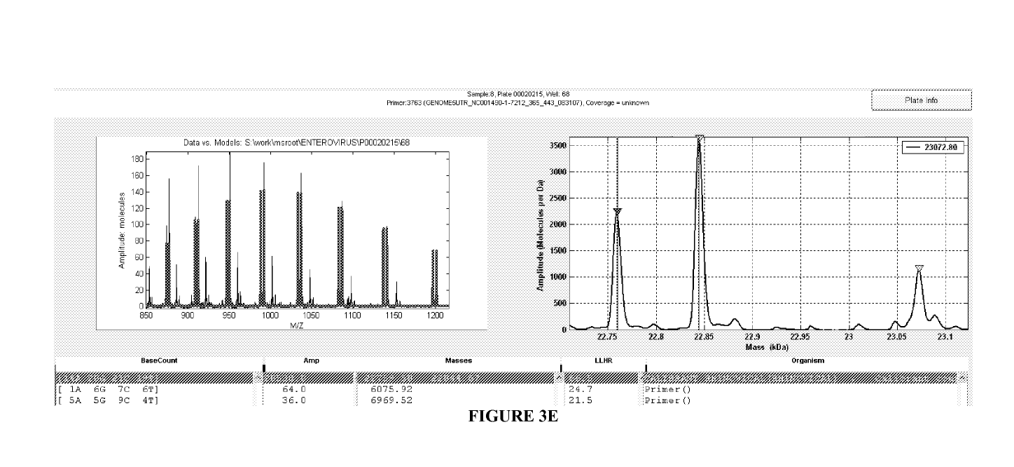

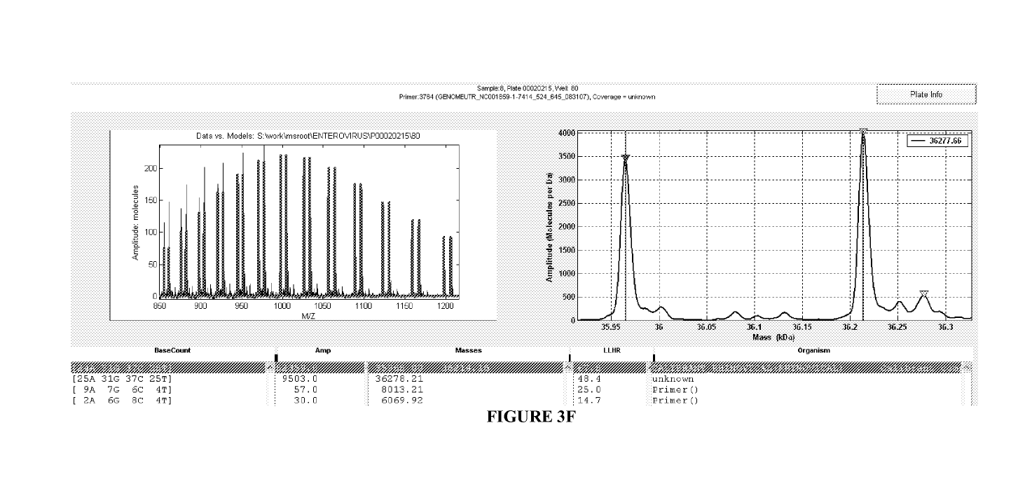

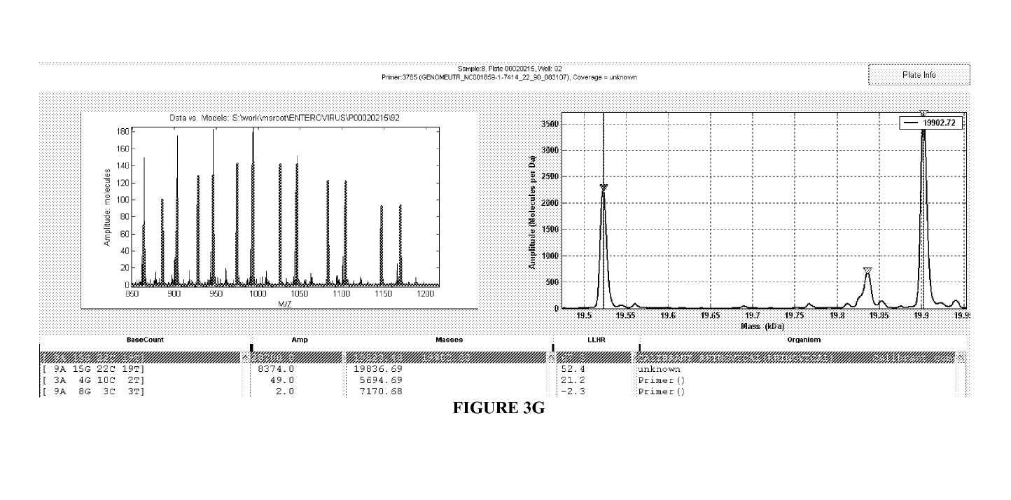

Mass spectrometry performed on amplification products generated using Primer pair 3759 in the assay described above revealed mass and base composition that gave a unique match to the base composition of a previously known Enterovirus calibrant indexed in the database. Primer pair 3759 amplifies calibrant and also a variant of the calibrant amplicon in which an additional A base is added to the sequence. The results are show in FIG. 3A. Results obtained using the Enterovirus calibrant with primer pairs 3758, 3760, and 3761, respectively, are shown in FIGS. 3B-D, whereas results obtained using the Rhinovirus calibrant with primer pairs 3763, 3764, and 3764, respectively, are shown in FIGS. 3E-G.

A dilution to extinction (DTE) analysis was performed to demonstrate the sensitivity of the primer pairs for detection against the positive control construct (IPC). The results are shown in Table 5. An “X” indicates that the target was detected with the indicated primer pair and the indicated copy number.

TABLE 5
Validation of Enterovirus Primer Pairs, DTE of Enterovirus and Rhinovirus
primer pairs against Positive Control Construct (IPC) RNA

| | Primer Pair | IPC Copies/rxn | | | | | | | | | |
|---|---|---|---|---|---|---|---|---|---|---|---|
| IPC | Number | 0 | 0.05 | 0.5 | 5 | 50 | 500 | 5000 | 50000 | 500000 | 5000000 [symbol] |
| Enterovirus | 3758 | | | | X | X | X | X | X | X | X |
| calibrant | 3759 | | | X | X | X | X | X | X | X | X |
| | 3760 | | | X | X | X | X | X | X | X | X |
| | 3761 | | X | X | X | X | X | X | X | X | X |
| Rhinovirus | 3763 | | | | X | X | X | X | X | X | X |
| Calibrant | 3764 | | | | X | X | X | X | X | X | X |
| | 3765 | | | | X | X | X | X | X | X | X |

[symbol]

indicates data missing or illegible when filed

### EXAMPLE 2

### Sample Preparation and PCR

Samples were processed to obtain viral genomic material using a Qiagen QIAamp Virus BioRobot MDx Kit (Valencia, Calif.). Resulting genomic material was amplified using an MJ Thermocycler Dyad unit (BioRad laboratories, Inc., Hercules, Calif.) and the amplicons were characterized on a Bruker Daltonics MicroTOF instrument (Billerica, Mass.). The resulting molecular mass measurements were converted to base compositions and were queried into a database having base compositions indexed with primer pairs and bioagents.

All PCR reactions were assembled in 50 μL reaction volumes in a 96-well microtiter plate format using a Packard MPII liquid handling robotic platform (Perkin Elmer, Boston, Mass.) and M.J. Dyad thermocyclers (BioRad, Inc., Hercules, Calif.). The PCR reaction mixture consisted of 4 units of Amplitaq Gold, 1× buffer II (Applied Biosystems, Foster City, Calif.), 1.5 mM MgCl₂, 0.4 M betaine, 800 μM dNTP mixture and 250 nM of each primer. The following typical PCR conditions were used: 95° C. for 10 minutes followed by 8 cycles of 95° C. for 30 seconds, 48° C. for 30 seconds, and 72° C. 30 seconds with the 48° C. annealing temperature increasing 0.9° C. with each of the eight cycles. The PCR was then continued for 37 additional cycles of 95° C. for 15 seconds, 56° C. for 20 seconds, and 72° C. 20 seconds. Those ordinarily skilled in the art will understand PCR reactions.

### EXAMPLE 3

### Solution Capture Purification of PCR Products for Mass Spectrometry with Ion Exchange Resin-Magnetic Beads

For solution capture of nucleic acids with ion exchange resin linked to magnetic beads, 25 μl of a 2.5 mg/mL suspension of BioClone amine terminated supraparamagnetic beads (San Diego, Calif.) were added to 25 to 50 μl of a PCR (or RT-PCR) reaction containing approximately 10 pM of an amplicon. The above suspension was mixed for approximately 5 minutes by vortexing or pipetting, after which the liquid was removed after using a magnetic separator. The beads containing bound PCR amplicon were then washed three times with 50 mM ammonium bicarbonate/50% MeOH or 100 mM ammonium bicarbonate/50% MeOH, followed by three more washes with 50% MeOH. The bound PCR amplicon was eluted with a solution of 25 mM piperidine, 25 mM imidazole, 35% MeOH which included peptide calibration standards.

### EXAMPLE 4

### Mass Spectrometry and Base Composition Analysis

The ESI-FTICR mass spectrometer is based on a Bruker Daltonics (Billerica, Mass.) Apex II 70e electrospray ionization Fourier transform ion cyclotron resonance mass spectrometer that employs an actively shielded 7 Tesla superconducting magnet. The active shielding constrains the majority of the fringing magnetic field from the superconducting magnet to a relatively small volume. Thus, components that might be adversely affected by stray magnetic fields, such as CRT monitors, robotic components, and other electronics, can operate in close proximity to the FTICR spectrometer. All aspects of pulse sequence control and data acquisition were performed on a 600 MHz Pentium II data station running Bruker's Xmass software under Windows NT 4.0 operating system. Sample aliquots, typically 15 μl, were extracted directly from 96-well microtiter plates using a CTC HTS PAL autosampler (LEAP Technologies, Carrboro, N.C.) triggered by the FTICR data station. Samples were injected directly into a 10 μl sample loop integrated with a fluidics handling system that supplies the 100 μl/hr flow rate to the ESI source. Ions were formed via electrospray ionization in a modified Analytica (Branford, Conn.) source employing an off axis, grounded electrospray probe positioned approximately 1.5 cm from the metalized terminus of a glass desolvation capillary. The atmospheric pressure end of the glass capillary was biased at 6000 V relative to the ESI needle during data acquisition. A counter-current flow of dry N₂ was employed to assist in the desolvation process. Ions were accumulated in an external ion reservoir comprised of an rf-only hexapole, a skimmer cone, and an auxiliary gate electrode, prior to injection into the trapped ion cell where they were mass analyzed. Ionization duty cycles>99% were achieved by simultaneously accumulating ions in the external ion reservoir during ion detection. Each detection event consisted of 1M data points digitized over 2.3 seconds. To improve the signal-to-noise ratio (S/N), 32 scans were co-added for a total data acquisition time of 74 seconds.

The ESI-TOF mass spectrometer is based on a Bruker Daltonics MicroTOF™. Ions from the ESI source undergo orthogonal ion extraction and are focused in a reflectron prior to detection. The TOF and FTICR are equipped with the same automated sample handling and fluidics described above. Ions are formed in the standard MicroTOF™ ESI source that is equipped with the same off-axis sprayer and glass capillary as the FTICR ESI source. Consequently, source conditions were the same as those described above. External ion accumulation was also employed to improve ionization duty cycle during data acquisition. Each detection event on the TOF was comprised of 75,000 data points digitized over 75 μs.

The sample delivery scheme allows sample aliquots to be rapidly injected into the electrospray source at high flow rate and subsequently be electrosprayed at a much lower flow rate for improved ESI sensitivity. Prior to injecting a sample, a bolus of buffer was injected at a high flow rate to rinse the transfer line and spray needle to avoid sample contamination/carryover. Following the rinse step, the autosampler injected the next sample and the flow rate was switched to low flow. Following a brief equilibration delay, data acquisition commenced. As spectra were co-added, the autosampler continued rinsing the syringe and picking up buffer to rinse the injector and sample transfer line. In general, two syringe rinses and one injector rinse were required to minimize sample carryover. During a routine screening protocol a new sample mixture was injected every 106 seconds. More recently a fast wash station for the syringe needle has been implemented which, when combined with shorter acquisition times, facilitates the acquisition of mass spectra at a rate of just under one spectrum/minute.

Raw mass spectra were post-calibrated with an internal mass standard and deconvoluted to monoisotopic molecular masses. Unambiguous base compositions were derived from the exact mass measurements of the complementary single-stranded oligonucleotides. Quantitative results are obtained by comparing the peak heights with an internal PCR calibration standard present in every PCR well at 500 molecules per well. Calibration methods are commonly owned and disclosed in U.S. Provisional Patent Application Ser. No. 60/545,425 which is incorporated herein by reference in entirety.

### EXAMPLE 5

### De Novo Determination of Base Composition of Amplicons Using Molecular Mass Modified Deoxynucleotide Triphosphates

Because the molecular masses of the four natural nucleobases have a relatively narrow molecular mass range (A=313.058, G=329.052, C=289.046, T=304.046, values in Daltons—See, Table 6), a persistent source of ambiguity in assignment of base composition may occur as follows: two nucleic acid strands having different base composition may have a difference of about 1 Da when the base composition difference between the two strands is G [symbol] A (−15.994) combined with C [symbol] T (+15.000). For example, one 99-mer nucleic acid strand having a base composition of A₂₇G₃₀C₂₁T₂₁ has a theoretical molecular mass of 30779.058 while another 99-mer nucleic acid strand having a base composition of A₂₆G₃₁C₂₂T₂₀ has a theoretical molecular mass of 30780.052 is a molecular mass difference of only 0.994 Da. A 1 Da difference in molecular mass may be within the experimental error of a molecular mass measurement and thus, the relatively narrow molecular mass range of the four natural nucleobases imposes an uncertainty factor in this type of situation. One method for removing this theoretical 1 Da uncertainty factor uses amplification of a nucleic acid with one mass-tagged nucleobase and three natural nucleobases.

Addition of significant mass to one of the 4 nucleobases (dNTPs) in an amplification reaction, or in the primers themselves, will result in a significant difference in mass of the resulting amplicon (greater than 1 Da) arising from ambiguities such as the G [symbol] A combined with C [symbol] T event (Table 6). Thus, the same G [symbol] A (−15.994) event combined with 5-Iodo-C [symbol] T (−110.900) event would result in a molecular mass difference of 126.894 Da. The molecular mass of the base composition A₂₇G₃₀5-Iodo-C₂₁T₂₁ (33422.958) compared with A₂₆G₃₁5-Iodo-C₂₂T₂₀, (33549.852) provides a theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀5-Iodo-C₂₁T₂₁. In contrast, the analogous amplification without the mass tag has 18 possible base compositions.

TABLE 6
Molecular Masses of Natural Nucleobases and the Mass-Modified
Nucleobase 5-Iodo-C and Molecular Mass Differences Resulting
from Transitions

| Nucleobase | Molecular Mass | Transition | Δ Molecular Mass |
|---|---|---|---|
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

Mass spectra of bioagent-identifying amplicons may be analyzed using a maximum-likelihood processor, such as is widely used in radar signal processing. This processor first makes maximum likelihood estimates of the input to the mass spectrometer for each primer by running matched filters for each base composition aggregate on the input data. This includes the response to a calibrant for each primer.

The algorithm emphasizes performance predictions culminating in probability-of-detection versus probability-of-false-alarm plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bacterial and viral bioagents and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of all system noise are factored into this two-stage maximum likelihood calculation. The processor reports the number of molecules of each base composition contained in the spectra. The quantity of amplicon corresponding to the appropriate primer set is reported as well as the quantities of primers remaining upon completion of the amplification reaction.

Base count blurring may be carried out as follows. Electronic PCR can be conducted on nucleotide sequences of the desired bioagents to obtain the different expected base counts that could be obtained for each primer pair. See for example, Schuler, Genome Res. 7:541-50, 1997; or the e-PCR program available from National Center for Biotechnology Information (NCBI, NIH, Bethesda, Md.). In one embodiment one or more spreadsheets from a workbook comprising a plurality of spreadsheets may be used (e.g., Microsoft Excel). First, in this example, there is a worksheet with a name similar to the workbook name; this worksheet contains the raw electronic PCR data. Second, there is a worksheet named “filtered bioagents base count” that contains bioagent name and base count; there is a separate record for each strain after removing sequences that are not identified with a genus and species and removing all sequences for bioagents with less than 10 strains. Third, there is a worksheet, “Sheet1” that contains the frequency of substitutions, insertions, or deletions for this primer pair. This data is generated by first creating a pivot table from the data in the “filtered bioagents base count” worksheet and then executing an Excel VBA macro. The macro creates a table of differences in base counts for bioagents of the same species, but different strains. One of ordinary skill in the art understands the additional pathways for obtaining similar table differences without undo experimentation.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by taking the most abundant strain's base type composition and adding it to the reference set and then the next most abundant strain's base type composition is added until the threshold is met or exceeded. The current set of data was obtained using a threshold of 55%, which was obtained empirically.

For each base count not included in the reference base count set for that bioagent, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260 (U.S. application Ser. No. 10/418,514) which is incorporated herein by reference in entirety.

### EXAMPLE 6

### Selection and Validation of Primers that Define Bioagent Identifying Amplicons for Cardioviruses

The Cardioviruses are most closely related to the Aphthoviruses and Erboviruses. The development of a series of primer pairs that target known members of this viral group is shown in Table 7 (Forward Primers) and Table 8 (Reverse Primers). In order to evaluate sequence conservation across this genus, all known Cardiovirus sequences available from Genbank were obtained. Fifty partial or complete genome sequences in Genbank were aligned to one another. Primers were chosen from each of the major segments. The most conserved primer pairs were found in the 5′-untranslated region of the genome. Primer pairs were chosen in this section of the genome for Cardiovirus coverage. Additional primer pairs were then selected to each of the major gene segments that target the specific sequences (Table 7).

It should be noted that primer pair 4102 targeting Cardioviruses was tested against a number of different isolates and synthetic controls. None of the templates produced the expected amplicons. The forward and reverse primer pairs for this region were redesigned and the same templates tested again, and this too failed in all tests. In contrast, for example, primer pair 4103, which targets an upstream region, worked against all targets.

TABLE 7
Forward Primers of Primer Pairs for Identification of Cardioviruses

| | | | | | | SEQ |
|---|---|---|---|---|---|---|
| | | | forward | forward primer | forward primer | ID |
| pp num | pp code | pp name | primer code | name | sequence | NO |
| 4102 | VIR4102 | CARDIOVIRUS5UTR_NC | VIR9312F | CARDIOVIRUS5UTR_{—} | TGGATGTCCAGAA | 60 |
| | | 001479-1- | | NC001479-1- | GGTACCCC | |
| | | 7835_688_812 | | 7835_688_708_F | | |
| 4103 | VIR4103 | CARDIOVIRUS5UTR_NC | VIR9314F | CARDIOVIRUS5UTR_{—} | TGCCAAAAGCCAC | 61 |
| | | 001479-1- | | NC001479-1- | GTGTATAAGA | |
| | | 7835_564_709 | | 7835_564_586_F | | |
| 4104 | VIR4104 | CARDIOVIRUS1C_NC001 | VIR9316F | CARDIOVIRUS1C_NC | TGGGTTACCGTGT | 62 |
| | | 479-1-7835_2568_2693 | | 001479-1- | GGCAGCT | |
| | | | | 7835_2568_2587_F | | |
| 4105 | VIR4105 | CARDIOVIRUS3D_NC00 | VIR9318F | CARDIOVIRUS3D_NC | TCAAGCCTGGCAA | 63 |
| | | 1479-1- | | 001479-1- | AGACAGGAT | |
| | | 7835_7377_7515 | | 7835_7377_7398_F | | |
| 4106 | VIR4106 | CARDIOVIRUS3D_NC00 | VIR9320F | CARDIOVIRUS3D_NC | TGAGAGTGTGGAG | 64 |
| | | 1479-1- | | 001479-1- | TACAGATGGAGG | |
| | | 7835_7673_7783 | | 7835_7673_7697_F | | |
| 4107 | VIR4107 | CARDIOVIRUS3AB_NC0 | VIR9322F | CARDIOVIRUS3AB_N | TGGCGAAGACGGT | 65 |
| | | 01479-1- | | C001479-1- | GAAGCAGATGG | |
| | | 7835_5587_5662 | | 7835_5587_5610_F | | |
| 4108 | VIR4108 | THEILOVIRUS5UTR_NC0 | VIR9324F | THEILOVIRUS5UTR_N | TGTGTGCCCTATTT | 66 |
| | | 01366-1- | | C001366-1- | GCACAGC | |
| | | 8101_1094_1218 | | 8101_1094_1114_F | | |
| 4109 | VIR4109 | THEILOVIRUS5UTR_NC0 | VIR9326F | THEILOVIRUS5UTR_N | TACTGCGATAGTG | 67 |
| | | 01366-1-8101_93_227 | | C001366-1- | CCACCCC | |
| | | | | 8101_93_112_F | | |
| 4110 | VIR4110 | THEILOVIRUS5UTR- | VIR9328F | THEILOVIRUS5UTR- | TGGACGATGATGT | 68 |
| | | 1A_NC001366-1- | | 1A_NC001366-1- | CTTCTGGCCT | |
| | | 8101_1195_1334 | | 8101_1195_1217_F | | |
| 4111 | VIR4111 | THEILOVIRUS2A- | VIR9330F | THEILOVIRUS2A- | TGTGCGCGGGTAC | 69 |
| | | 2B_NC001366-1- | | 2B_NC001366-1- | CATGC | |
| | | 8101_4166_4271 | | 8101_4166_4183_F | | |
| 4112 | VIR4112 | THEILOVIRUS2C_NC001 | VIR9332F | THEILOVIRUS2C_NC0 | TATGGCTCACCTG | 70 |
| | | 366-1-8101_5228_5345 | | 01366-1- | GAAAGGAAAGG | |
| | | | | 8101_5228_5251_F | | |
| 4113 | VIR4113 | THEILOVIRUS3D_NC001 | VIR9334F | THEILOVIRUS3D_NC0 | TCACTGCATTTGG | 71 |
| | | 366-1-8101_7139_7248 | | 01366-1- | GGCAGACAGC | |
| | | | | 8101_7139_7161_F | | |

TABLE 8
Reverse Primers of Primer Pairs for Identification of Cardioviruses

| | | | | | | SEQ |
|---|---|---|---|---|---|---|
| | | | reverse | reverse | reverse primer | ID |
| pp num | pp code | pp name | primer code | primer name | sequence | NO |
| 4102 | VIR4102 | CARDIOVIRUS5UTR_NC | VIR9313R | CARDIOVIRUS5UTR_{—} | TGAAAACCACGACCACG | 72 |
| | | 001479-1- | | NC001479-1- | TGGTT | |
| | | 7835_688_812 | | 7835_791_812_R | | |
| 4103 | VIR4103 | CARDIOVIRUS5UTR_NC | VIR9315R | CARDIOVIRUS5UTR_{—} | TGGGGTACCTTCTGGGC | 73 |
| | | 001479-1- | | NC001479-1- | AT | |
| | | 7835_564_709 | | 7835_691_709_R | | |
| 4104 | VIR4104 | CARDIOVIRUS1C_NC00 | VIR9317R | CARDIOVIRUS1C_NC | TGGGGCAGGTGAGATG | 74 |
| | | 1479-1- | | 001479-1- | GGCA | |
| | | 7835_2568_2693 | | 7835_2674_2693_R | | |
| 4105 | VIR4105 | CARDIOVIRUS3D_NC00 | VIR9319R | CARDIOVIRUS3D_NC | TCATGACAGGTCGATAC | 75 |
| | | 1479-1- | | 001479-1- | AGAGGGC | |
| | | 7835_7377_7515 | | 7835_7492_7515_R | | |
| 4106 | VIR4106 | CARDIOVIRUS3D_NC00 | VIR9321R | CARDIOVIRUS3D__NC | TGTCCGCAGTATGACGA | 76 |
| | | 1479-1- | | 001479-1- | CCGTG | |
| | | 7835_7673_7783 | | 7835_7762_7783_R | | |
| 4107 | VIR4107 | CARDIOVIRUS3AB_NC0 | VIR9323R | CARDIOVIRUS3AB_N | TAAGGTCCCTGCTCTTGC | 77 |
| | | 01479-1- | | C001479-1- | TCATCCA | |
| | | 7835_5587_5662 | | 7835_5638_5662_R | | |
| 4108 | VIR4108 | THEILOVIRUS5UTR_NC | VIR9325R | THEILOVIRUS5UTR_N | TAGGCCAGAAGACATCA | 78 |
| | | 001366-1- | | C001366-1- | TCGTCCA | |
| | | 8101_1094_1218 | | 8101_1195_1218_R | | |
| 4109 | VIR4109 | THEILOVIRUS5UTR_NC | VIR9327R | THEILOVIRUS5UTR_N | TGGGCCGGAAAATGCTG | 79 |
| | | 001366-1- | | C001366-1- | AC | |
| | | 8101_93_227 | | 8101_209_227_R | | |
| 4110 | VIR4110 | THEILOVIRUS5UTR- | VIR9329R | THEILOVIRUS5UTR- | TGACTGGGAGTTACTCT | 80 |
| | | 1A_NC001366-1- | | 1A_NC001366-1- | TGTCAGATGA | |
| | | 8101_1195_1334 | | 8101_1308_1334_R | | |
| 4111 | VIR4111 | THEILOVIRUS2A- | VIR9331R | THEILOVIRUS2A- | TAGCACCCCACCTTGTG | 81 |
| | | 2B_NC001366-1- | | 2B_NC001366-1- | GC | |
| | | 8101__4166_4271 | | 8101_4253_4271_R | | |
| 4112 | VIR4112 | THEILOVIRUS2C_NC00 | VIR9333R | THEILOVIRUS2C_NCO | TCGCCTATCAACAGCTG | 82 |
| | | 1366-1- | | 01366-1- | GGTA | |
| | | 8101_5228_5345 | | 8101_5325_5345_R | | |
| 4113 | VIR4113 | THEILOVIRUS3D_NC00 | VIR9335R | THEILOVIRUS3D_NC0 | TCCAATCAACATCCGGG | 83 |
| | | 1366-1- | | 01366-1- | TCAGTTCC | |
| | | 8101_7139_7248 | | 8101_7224_7248_R | | |

### EXAMPLE 7

### Selection and Validation of Primers that Define Bioagent Identifying Amplicons for Hepatoviruses

Tables 9 and 10 show forward and reverse primers of primer pairs for identification of Cardioviruses, respectively.

TABLE 9
Forward Primers of Primer Pairs for Identification of Hepatoviruses

| | | | forward | | | |
|---|---|---|---|---|---|---|
| | | | primer | forward primer | forward primer | SEQ ID |
| pp num | pp code | pp name | code | name | sequence | NO |
| 3033 | VIR3033 | HAV_NC001489- | VIR7306F | HAV_NC001489- | TCAACCACAGTTT | 84 |
| | | 735- | | 735 | CTACAGAACAGAA | |
| | | 7418_1498_161 | | 7418_1498_1529_F | TGTTCC | |
| 3034 | VIR3034 | HAV_NC001489- | VIR7308F | HAV_NC001489- | TGTGATGACAGTT | 85 |
| | | 735_{—} | | 735- | GAAATTAGGAAAC | |
| | | 7418__4206_4323 | | 7418_4206_4239_F | AAAATATG | |
| 3035 | VIR3035 | HAV_NC001489- | VIR7310F | HAV_NC001489- | TGAAAGTCAGAGA | 86 |
| | | 735- | | 735- | ATGATGAAAGTGG | |
| | | 7418_5205_5270_F | | 7418_5205_5231_F | A | |
| 3036 | VIR3036 | HAV__NC001489- | VIR7312F | HAV_NC001489- | TGTTCAATGAATG | 87 |
| | | 735- | | 735- | TAGTCTCCAAAAC | |
| | | 7418_5242_5328 | | 7418_5242_5269_F | GC | |
| 3037 | VIR3037 | HAV_NC001489- | VIR7314F | HAV_NC001489- | TGCTCAGTGGTTT | 88 |
| | | 735- | | 735- | CTTTTATGCATGG | |
| | | 7418_6522__602 | | 7418_6522_6548_F | G | |
| 3038 | VIR3038 | HAV_NC001489- | VIR7316F | HAV_NC001489- | TCAGTCCTGCTTG | 89 |
| | | 735- | | 735- | GAGAAAGAGATGA | |
| | | 7418_6582_6659 | | 7418_6582_6614_F | TAGAATA | |
| 3039 | VIR3039 | HAV_NC001489- | VIR7318F | HAV_NC001489- | TGTGTGGACTTTT | 90 |
| | | 735- | | 735- | GAGATGGATGCTG | |
| | | 7418_2628_2714 | | 7418_2628_2654_F | G | |
| 3040 | VIR3040 | HAV_NC001489- | VIR7320F | HAV_NC001489- | TGGATGTGTGAGA | 91 |
| | | 735- | | 735- | TGGGTCATGAATG | |
| | | 7418_4623_4743 | | 7418_4623_4649_F | C | |
| 3041 | VIR3041 | HAV_NC001489- | VIR7322F | HAV_NC001489- | TGCAGTGGCTGAG | 92 |
| | | 735- | | 735- | TTTTTCCAGTCTT | |
| | | 7418_4293_4404 | | 7418_4293_4322_F | TTCC | |
| 3042 | VIR3042 | HAV_NC001489- | VIR7324F | HAV_NC001489- | TGTTGGGAGTGGT | 93 |
| | | 735- | | 735- | CTTGACCACAT | |
| | | 7418_33_146 | | 7418_33_56_F | | |
| 3043 | VIR3043 | HAV_NC001489- | VIR7326F | HAV_NC001489- | TCCAGGGATGTGT | 94 |
| | | 735- | | 735- | GGTGGGGC | |
| | | 7418_5061_5139 | | 7418_5061_5081_F | | |
| 3044 | VIR3044 | HAV_NC001489- | VIR7328F | HAV_NC001489- | TGCTAGGTTTACGG | 95 |
| | | 735- | | 735- | ATTTGGAGCTGCAT | |
| | | 7418_684_822 | | 7418_684_713_F | GG | |
| 3045 | VIR3045 | HAV_NC001489- | VIR7330F | HAV_NC001489- | TTGAAATACCATTC | 96 |
| | | 735- | | 735- | TTATGCAAGATTTG | |
| | | 7418_361_431 | | 7418_361_390_F | GC | |
| 3046 | VIR3046 | HAV_NC001489- | VIR7332F | HAV_NC001489- | TATGTTTTGTTTTT | 97 |
| | | 735- | | 735- | GGAGAGGAGACCTT | |
| | | 7418_1053_1119 | | 7418_1053_1082_F | GT | |

TABLE 10
Reverse Primers of Primer Pairs for Identification of Cardioviruses

| | | | reverse | | | SEQ |
|---|---|---|---|---|---|---|
| | | | primer | reverse primer | reverse primer | ID |
| pp num | pp code | pp name | code | name | sequence | NO |
| 3033 | VIR3033 | HAV_NC001489- | VIR7307R | HAV_NC001489- | TGGTGCTTGGACTC | 98 |
| | | 735- | | 735- | CTGAAACATCCAT | |
| | | 7418_1498_1617 | | 7418_1591_1617_R | | |
| 3034 | VIR3034 | HAV_NC001489- | VIR7309R | HAV_NC001489- | TGGAAAAGACTGGA | 99 |
| | | 735- | | 735- | AAAACTCAGCTACT | |
| | | 7418_4206_4323 | | 7418_4293_4323_R | GCA | |
| 3035 | VIR3035 | HAV_NC001489- | VIR7311R | HAV_NC001489- | TGCGTTTTGGAGAC | 100 |
| | | 735- | | 735- | TACATTCATTGAAC | |
| | | 7418_5205_5270 | | 7418_5242_5270_R | A | |
| 3036 | VIR3036 | HAV_NC001489- | VIR7313R | HAV_NC001489- | TGCAGGAAAATTAA | 101 |
| | | 735- | | 735- | TCATGTTTTTATCA | |
| | | 7418_5242_5328 | | 7418_5296_5328_R | ATGTG | |
| 3037 | VIR3037 | HAV_NC001489- | VIR7315R | HAV_NC001489- | TCTTTCTCCAAACA | 102 |
| | | 735- | | 735- | GGACTGAACAAAAT | |
| | | 7418_6522_6602 | | 7418_6575_6602_R | | |
| 3038 | VIR3038 | HAV_NC001489- | VIR7317R | HAV_NC001489- | TGGTCATAAAACCT | 103 |
| | | 735- | | 735- | CATTCTCCACCAAT | |
| | | 7418_6582_6659 | | 7418_6628_6659_R | CATA | |
| 3039 | VIR3039 | HAV_NC001489- | VIR7319R | HAV_NC001489- | TCATCCTTCATTTC | 104 |
| | | 735- | | 735- | TGTCCATTTTTCAT | |
| | | 7418_2628_2714 | | 7418_2683_2714_R | CATT | |
| 3040 | VIR3040 | HAVNC001489- | VIR7321R | HAV_NC001489- | TCCTCTATTGAAAT | 105 |
| | | 735- | | 735- | AAAACTCCATCATT | |
| | | 7418_4623_4743 | | 7418_4708_4743_R | TCATAATC | |
| 3041 | VIR3041 | HAV_NC001489- | VIR7323R | HAV_NC001489- | TGCAGCTCCAACAG | 106 |
| | | 735- | | 735- | | |
| | | 7418_4293_4404 | | 7418_4384_4404_R | CAACCCA | |
| 3042 | VIR3042 | HAV_NC001489- | VIR7325R | HAV_NC001489- | TCCACAGAAGTAAA | 107 |
| | | 735- | | 735- | ATAAGAAGCACCAG | |
| | | 7418_33_146 | | 7418_118_146_R | T | |
| 3043 | VIR3043 | HAV_NC001489- | VIR7327R | HAV_NC001489- | TCCAGCAACATGAA | 108 |
| | | 735- | | 735- | TGCCCAAAATGGCA | |
| | | 7418_5061_5139 | | 7418_5107_5139_R | TTCTG | |
| 3044 | VIR3044 | HAV_NC001489- | VIR7329R | HAV_NC001489- | TAAAGACATTTTGG | 109 |
| | | 735- | | 735- | CTCTTGCATCTTCA | |
| | | 7418_684_822 | | 7418_790_822_R | TAATT | |
| 3045 | VIR3045 | HAV_NC001489- | VIR7331R | HAV_NC001489- | TGCTGGAATGGTGT | 110 |
| | | 735- | | 735- | TGGATTTATCTGAA | |
| | | 7418_361_431 | | 7418_403_431_R | C | |
| 3046 | VIR3046 | HAV_NC001489- | VIR7333R | HAV_NC001489- | TGAATGATATTTGG | 111 |
| | | 735- | | 735- | TGGGAAAGACTTGA | |
| | | 7418_1053_1119 | | 7418_1090_1119_R | AA | |

Table 11 shows primer pairs with coverage of known hepatovirus species including human HAV, Simian HAV and Avian encephalomyelitis virus. Calibrant standards were developed for each of the primer pairs and tested in limiting dilution experiments. As indicated in the detection sensitivity column, all primers tested performed to the limits of PCR (5-20 copies per well).

TABLE 11
Validation of Hepatovirus Primer Pairs

| | Primer | Detection |
|---|---|---|
| Specificity | Pair Code | sensitivity |
| Human/Simian | VIR3033 | 20 |
| Human/Simian | VIR3034 | 80 |
| Human/Simian | VIR3035 | 5 |
| Human/Simian | VIR3036 | 5 |
| Human/Simian | VIR3037 | 10 |
| Human/Simian | VIR3038 | 5 |
| Human/Simian | VIR3039 | 20 |
| Human/Simian | VIR3040 | 20 |
| Human/Simian | VIR3041 | 20 |
| Human/Simian | VIR3042 | 20 |
| Avian/human/simian | VIR3043 | 5 |
| Avian/human/simian | VIR3044 | 20 |
| Avian/human/simian | VIR3045 | 10 |
| Avian/human/simian | VIR3046 | 10 |

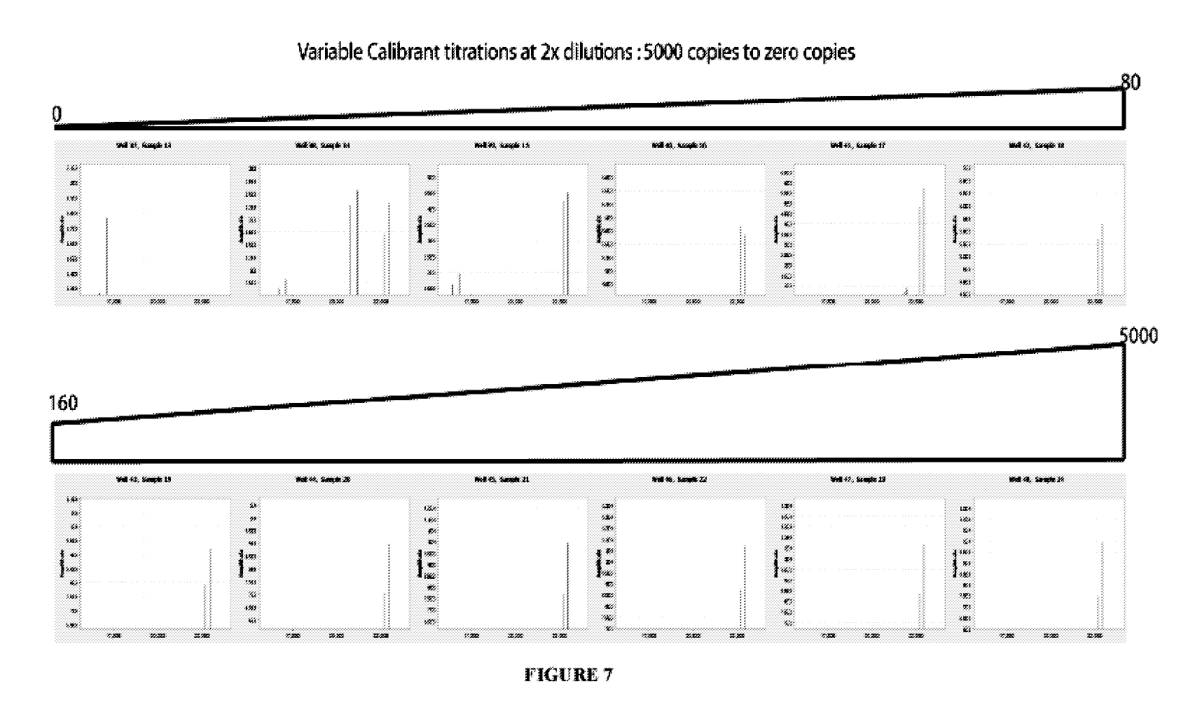

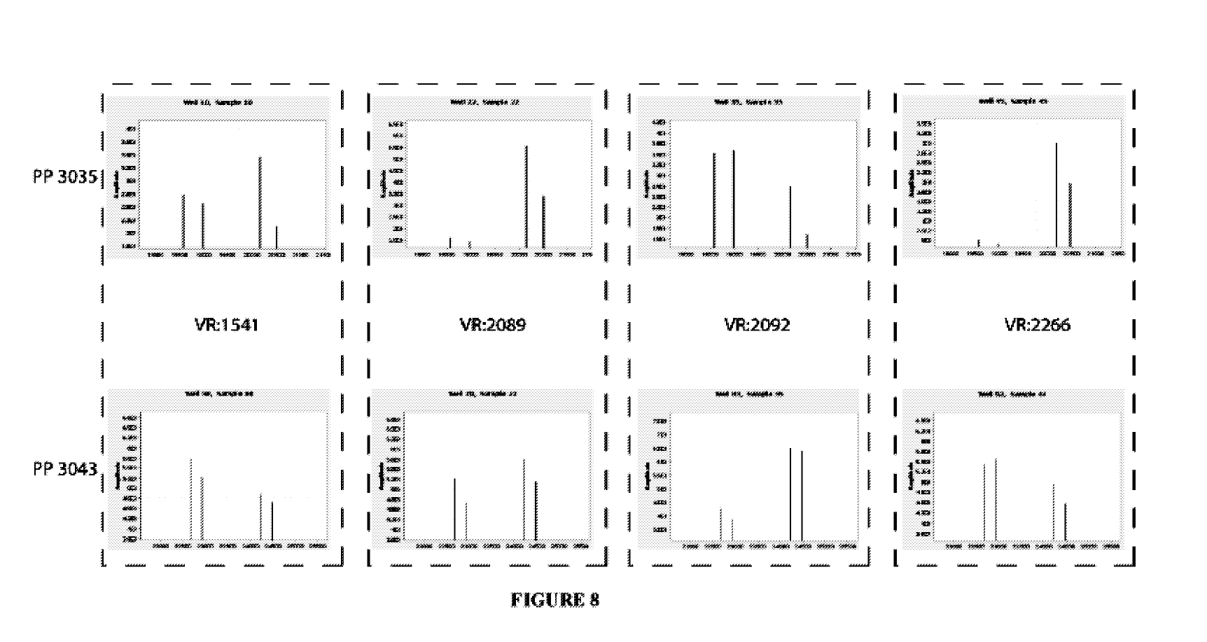

FIGS. 7 and 8 show Hepatovirus primer testing results. FIG. 7 shows 2× limiting dilutions of the RNA calibrant standard tested against one of the broad primers (PP3043) from Tables 9 and 10. Based on the detection of the calibrant, this primer was sensitive down to 10 copies of input RNA per well. While the calibrant was detected at 5 copies as well, there was a strong primer dimer, indicating weaker binding to the target. FIG. 8 shows detection of four different ATCC HAV stocks (VR: 1541, 2089, 2092 and 2266) using two of the primer pairs, PP3035 and 3043.

### EXAMPLE 8

### Analysis of a Cardiovirus Targeted Primer Pair Against Known EMCV Containing Samples

Results from testing a Cardiovirus targeted primer pair against known encephalomyocarditis virus (EMCV) containing samples (PP4106) are summarized in Table 12. More specifically, samples 2-8 were all positive for EMCV in the presence of large excess of various cell line nucleic acid extracts. Samples 9-12 were negative controls with just the cell background. Samples 13-15 were unknown samples that were shown to be negative for EMCV by this assay. Table 12 also shows the details of the spike levels and where detections were made. All positive EMCV samples detected positive at both dilutions and all the negatives (Cell line controls) were negative. There was 100% concordance in this analysis.

TABLE 12
Validation of Cardiovirus Primer Pair 4106

| T5000 | | Conc. | | Concor- | Results |
|---|---|---|---|---|---|
| Sample ID | Virus | [copies/ml] | Medium | dance | from |
| S1 | Blank | NA | NA | Yes | Negative |
| S2 | EMCV | 20.000* | Vero | Yes | EMCV |
| | | | | | (Weak |
| | | | | | detection) |
| S3 | EMCV | 20.000* | MRC-5 | Yes | EMCV |
| S4 | EMCV | 20.000* | CHO | Yes | EMCV |
| S5 | EMCV | 200.000* | Vero | Yes | EMCV |
| S6 | EMCV | 200.000* | MRC-5 | Yes | EMCV |
| S7 | EMCV | 200.000* | CHO | Yes | EMCV |
| S8 | X-MuLV/ | 50.000*/ | Vero | No | |
| | EMCV | 50.000* | | ND | EMCV |
| S9 | — | — | Vero | Yes | Negative |
| S10 | — | — | MRC-5 | Yes | Negative |
| S11 | — | — | CHO | Yes | Negative |
| S12 | — | — | 324-K | Yes | Negative |
| S13 | Plasma | — | Plasma | | Negative |
| | product | | inter- | | |
| | intermediate 1 | | mediate | | |
| S14 | Plasma | — | Plasma | | Negative |
| | product | | inter- | | |
| | intermediate 2 | | mediate | | |
| S15 | Plasma | 200 | Plasma | MMV | MMV |
| | product | | inter- | | (181) |
| | intermediate 1 | | mediate | | |
| | spiked with | | | | |
| | MMV | | | | |
| | (extraction | | | | |
| | control) | | | | |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, internet web sites, and the like) cited in the present application is incorporated herein by reference in its entirety.

What is claimed is:

1. A composition, comprising at least one purified oligonucleotide primer pair that comprises forward and reverse primers, wherein said primer pair comprises nucleic acid sequences that are substantially complementary to nucleic acid sequences of two or more different bioagents belonging to the Picornaviridae family, wherein said primer pair is configured to produce amplicons comprising different base compositions that correspond to said two or more different bioagents.

2. The composition of claim 1, wherein said primer pair is configured to hybridize with conserved regions of said two or more different bioagents and flank variable regions of said two or more different bioagents.

3. The composition of claim 1, wherein said forward and reverse primers are about 15 to 35 nucleobases in length, and wherein the forward primer comprises at least 70%, at least 80%, at least 90%, at least 95%, or at least 100% sequence identity with a sequence selected from the group consisting of SEQ ID NOS: 2-30, 60-71 and 84-97, and the reverse primer comprises at least 70% sequence identity with a sequence selected from the group consisting of SEQ ID NOS: 31-59, 72-83, and 98-111.

4. The composition of claim 1, wherein said primer pair is selected from the group of primer pair sequences consisting of: SEQ ID NOS: 2:31, 3:32, 4:33, 5:34, 6:35, 7:36, 8:37, 9:38, 10:39, 11:40, 12:41, 13:42, 4:43, 15:44, 16:45, 17:46, 18:47, 19:48, 20:49, 21:50, 22:51, 23:52, 24:53, 25:54, 26:55, 27:56, 28:57, 29:58, 30:59, 60:72, 61:73, 62:74, 63:75, 64:76, 65:77, 66:78, 67:79, 68:80, 69:81, 70:82, 71:83, 84:98, 85:99, 86:100, 87:101, 88:102, 89:103, 90:104, 91:105, 92:106, 93:107, 94:108, 95:109, 96:110, and 97:111.

5. The composition of claim 1, wherein said forward and reverse primers are about 15 to 35 nucleobases in length, and wherein:
the forward primer comprises at least 70%, at least 80%, at least 90%, at least 95%, or at least 100% sequence identity with the sequence of SEQ ID NO: 2, and the reverse primer comprises at least 70%, at least 80%, at least 90%, at least 95%, or at least 100% sequence identity with the sequence of SEQ ID NO: 31;
the forward primer comprises at least 70%, at least 80%, at least 90%, at least 95%, or at least 100% sequence identity with the sequence of SEQ ID NO: 3, and the reverse primer comprises at least 70%, at least 80%, at least 90%, at least 95%, or at least 100% sequence identity with the sequence of SEQ ID NO: 32;
the forward primer comprises at least 70%, at least 80%, at least 90%, at least 95%, or at least 100% sequence identity with the sequence of SEQ ID NO: 10, and the reverse primer comprises at least 70%, at least 80%, at least 90%, at least 95%, or at least 100% sequence identity with the sequence of SEQ ID NO: 39;
the forward primer comprises at least 70%, at least 80%, at least 90%, at least 95%, or at least 100% sequence identity with the sequence of SEQ ID NO: 4, and the reverse primer comprises at least 70%, at least 80%, at least 90%, at least 95%, or at least 100% sequence identity with the sequence of SEQ ID NO: 33;
the forward primer comprises at least 70%, at least 80%, at least 90%, at least 95%, or at least 100% sequence identity with the sequence of SEQ ID NO: 5, and the reverse primer comprises at least 70%, at least 80%, at least 90%, at least 95%, or at least 100% sequence identity with the sequence of SEQ ID NO: 34;
the forward primer comprises at least 70%, at least 80%, at least 90%, at least 95%, or at least 100% sequence identity with the sequence of SEQ ID NO: 7, and the reverse primer comprises at least 70%, at least 80%, at least 90%, at least 95%, or at least 100% sequence identity with the sequence of SEQ ID NO: 36;
the forward primer comprises at least 70%, at least 80%, at least 90%, at least 95%, or at least 100% sequence identity with the sequence of SEQ ID NO: 61, and the reverse primer comprises at least 70%, at least 80%, at least 90%, at least 95%, or at least 100% sequence identity with the sequence of SEQ ID NO: 73; and/or,
the forward primer comprises at least 70%, at least 80%, at least 90%, at least 95%, or at least 100% sequence identity with the sequence of SEQ ID NO: 64, and the reverse primer comprises at least 70%, at least 80%, at least 90%, at least 95%, or at least 100% sequence identity with the sequence of SEQ ID NO: 76.

6. The composition of claim 1, wherein said different base compositions identify said two or more different bioagents at genus, species, or sub-species levels.

7. The composition of claim 1, wherein said two or more amplicons are 45 to 200 nucleobases in length.

8. A kit comprising the composition of claim 1.

9. The composition of claim 1, wherein said different bioagents are selected from the group consisting of: Enterovirus A species, Enterovirus B species, Enterovirus C species, Enterovirus D species, Poliovirus species, Rhinovirus genus, Rhinovirus A species, Rhinovirus B species, Coxsackievirus genus, Coxsackievirus A species, Coxsackievirus B species, Coxsackievirus C species, Porcine enterovirus species, Bovine enterovirus species, Hepatovirus genus, Cardiovirus genus, or combinations thereof.

10. The composition of claim 1, wherein said primer pair is configured to hybridize with one or more nucleic acid sequences selected from the group consisting of: WTPV1, WTHRV14, WTCVB3, PCV305, PV5′HRV14, GENOME5UTR, GENOMEUTR, POLIO5UTR, POLIO3D, POLIO3C, COXA3D, COXA2C, COXB3D, COXB2C, COXB3C, COXC3D, HRVA3D, HRVA2C, HRVBVP1, HRVB2B, HAV, CARDIOVIRUS5UTR, CARDIOVIRUS1C, CARDIOVIRUS3D, CARDIOVIRUS3AB, THEILOVIRUS5UTR, THEILOVIRUS5UTR-1A, THEILOVIRUS2A-2B, THEILOVIRUS2C, and THEILOVIRUS3D nucleic acids.

11. The composition of claim 1, wherein a non-templated T residue on the 5′-end of said forward and/or reverse primer is removed.

12. The composition of claim 1, wherein said forward and/or reverse primer further comprises a non-templated T residue on the 5′-end.

13. The composition of claim 1, wherein said forward and/or reverse primer comprises at least one molecular mass modifying tag.

14. The composition of claim 1, wherein said forward and/or reverse primer comprises at least one modified nucleobase.

15. The composition of claim 14, wherein said modified nucleobase is 5-propynyluracil or 5-propynylcytosine.

16. The composition of claim 14, wherein said modified nucleobase is a mass modified nucleobase.

17. The composition of claim 16, wherein said mass modified nucleobase is 5-Iodo-C.

18. The composition of claim 14, wherein said modified nucleobase is a universal nucleobase.

19. The composition of claim 18, wherein said universal nucleobase is inosine.

20. A kit, comprising at least one purified oligonucleotide primer pair that comprises forward and reverse primers that are about 20 to 35 nucleobases in length, and wherein said forward primer comprises at least 70%, at least 80%, at least 90%, at least 95%, or at least 100% sequence identity with a sequence selected from the group consisting of SEQ ID NOS: 2-30, 60-71, and 84-97, and said reverse primer comprises at least 70% sequence identity with a sequence selected from the group consisting of SEQ ID NOS: 31-59, 72-83 and 98-111.

21. A method of determining a presence of a picornavirus in at least one sample, the method comprising:
(a) amplifying one or more segments of at least one nucleic acid from said sample using at least one purified oligonucleotide primer pair that comprises forward and reverse primers that are about 20 to 35 nucleobases in length, and wherein said forward primer comprises at least 70%, at least 80%, at least 90%, at least 95%, or at least 100% sequence identity with a sequence selected from the group consisting of SEQ ID NOs: 2-30, 60-71, and 84-97, and said reverse primer comprises at least 70% sequence identity with a sequence selected from the group consisting of SEQ ID NOs: 31-59, 72-83, and 98-111 to produce at least one amplification product; and
(b) detecting said amplification product, thereby determining said presence of said picornavirus in said sample.

22. The method of claim 21, wherein (a) comprises amplifying said one or more segments of said at least one nucleic acid from at least two samples obtained from different geographical locations to produce at least two amplification products, and (b) comprises detecting said amplification products, thereby tracking an epidemic spread of said picornavirus.

23. The method of claim 21, wherein (b) comprises determining an amount of said picornavirus in said sample.

24. The method of claim 21, wherein (b) comprises detecting a molecular mass of said amplification product.

25. The method of claim 21, wherein (b) comprises determining a base composition of said amplification product, wherein said base composition identifies the number of A residues, C residues, T residues, G residues, U residues, analogs thereof and/or mass tag residues thereof in said amplification product, whereby said base composition indicates the presence of picornavirus in said sample or identifies said picornavirus in said sample.

26. The method of claim 25, comprising comparing said base composition of said amplification product to calculated or measured base compositions of amplification products of one or more known picornaviruses present in a database with the proviso that sequencing of said amplification product is not used to indicate the presence of or to identify said picornavirus, wherein a match between said determined base composition and said calculated or measured base composition in said database indicates the presence of or identifies said picornavirus.

27. A method of identifying one or more picornavirus bioagents in a sample, the method comprising:
(a) amplifying two or more segments of a nucleic acid from said one or more picornavirus bioagents in said sample with two or more oligonucleotide primer pairs to obtain two or more amplification products;
(b) determining two or more molecular masses and/or base compositions of said two or more amplification products; and
(c) comparing said two or more molecular masses and/or said base compositions of said two or more amplification products with known molecular masses and/or known base compositions of amplification products of known picornavirus bioagents produced with said two or more primer pairs to identify said one or more picornavirus bioagents in said sample.

28. The method of claim 27, comprising identifying said one or more picornavirus bioagents in said sample using three, four, five, six, seven, eight or more primer pairs.

29. The method of claim 27, wherein said one or more picornavirus bioagents in said sample cannot be identified using a single primer pair of said two or more primer pairs.

30. The method of claim 27, comprising obtaining said two or more molecular masses of said two or more amplification products via mass spectrometry.

31. The method of claim 27, comprising calculating said two or more base compositions from said two or more molecular masses of said two or more amplification products.

32. The method of claim 27, wherein said picornavirus bioagents are selected from the group consisting of: an Enterovirus genus, a Rhinovirus genus, a Hepatovirus genus, a Cardiovirus genus, an Aphthovirus genus, a Parechovirus genus, an Erbovirus genus, a Kobuvirus genus, a Teschovirus genus, a species thereof, a sub-species thereof, and combinations thereof.

33. The method of claim 27, wherein said two or more primer pairs comprise two or more purified oligonucleotide primer pairs that each comprise forward and reverse primers that are about 20 to 35 nucleobases in length, and wherein said forward primers comprise at least 70%, at least 80%, at least 90%, at least 95%, or at least 100% sequence identity with a sequence selected from the group consisting of SEQ ID NOS: 2-30, 60-71, and 84-97, and said reverse primers comprise at least 70% sequence identity with a sequence selected from the group consisting of SEQ ID NOS: 31-59, 72-83, and 98-111 to obtain an amplification product.

34. The method of claim 27, wherein said primer pairs are selected from the group of primer pair sequences consisting of: SEQ ID NOS: 2:31, 3:32, 4:33, 5:34, 6:35, 7:36, 8:37, 9:38, 10:39, 11:40, 12:41, 13:42, 4:43, 15:44, 16:45, 17:46, 18:47, 19:48, 20:49, 21:50, 22:51, 23:52, 24:53, 25:54, 26:55, 27:56, 28:57, 29:58, 30:59, 60:72, 61:73, 62:74, 63:75, 64:76, 65:77, 66:78, 67:79, 68:80, 69:81, 70:82, 71:83, 84:98, 85:99, 86:100, 87:101, 88:102, 89:103, 90:104, 91:105, 92:106, 93:107, 94:108, 95:109, 96:110, and 97:111.

35. The method of claim 27, wherein said determining said two or more molecular masses and/or base compositions is conducted without sequencing said two or more amplification products.

36. The method of claim 27, wherein said one or more picornavirus bioagents in said sample cannot be identified using a single primer pair of said two or more primer pairs.

37. The method of claim 27, wherein said one or more picornavirus bioagents in a sample are identified by comparing three or more molecular masses and/or base compositions of three or more amplification products with a database of known molecular masses and/or known base compositions of amplification products of known picornavirus bioagents produced with said three or more primer pairs.

38. The method of claim 27, wherein said two or more segments of said nucleic acid are amplified from a single gene.

39. The method of claim 27, wherein said two or more segments of said nucleic acid are amplified from different genes.

40. The method of claim 27, wherein members of said primer pairs hybridize to conserved regions of said nucleic acid that flank a variable region.

41. The method of claim 40, wherein said variable region varies between at least two of said picornavirus bioagents.

42. The method of claim 40, wherein said variable region uniquely varies between at least five of said picornavirus bioagents.

43. The method of claim 27, wherein said two or more amplification products obtained in (a) comprise major classification and subgroup identifying amplification products.

44. The method of claim 43, comprising comparing said molecular masses and/or said base compositions of said two or more amplification products to calculated or measured molecular masses or base compositions of amplification products of known picornavirus bioagents in a database comprising genus specific amplification products, species specific amplification products, strain specific amplification products or nucleotide polymorphism specific amplification products produced with said two or more oligonucleotide primer pairs, wherein one or more matches between said two or more amplification products and one or more entries in said database identifies said one or more picornavirus bioagents, classifies a major classification of said one or more picornavirus bioagents, and/or differentiates between subgroups of known and unknown picornavirus bioagents in said sample.

45. The method of claim 44, wherein said major classification of said one or more picornavirus bioagents comprises a genus or species classification of said one or more picornavirus bioagents.

46. The method of claim 44, wherein said subgroups of known and unknown picornavirus bioagents comprise family, strain and nucleotide variations of said one or more picornavirus bioagents.

47. A system, comprising:
(a) a mass spectrometer configured to detect one or more molecular masses of amplicons produced using at least one purified oligonucleotide primer pair that comprises forward and reverse primers, wherein said primer pair comprises nucleic acid sequences that are substantially complementary to nucleic acid sequences of two or more different picornavirus bioagents; and
(b) a controller operably connected to said mass spectrometer, said controller configured to correlate said molecular masses of said amplicons with one or more picornavirus bioagent identities.

48. The system of claim 47, wherein said picornavirus bioagent identities are at genus, species, and/or sub-species levels.

49. The system of claim 47, wherein said forward and reverse primers are about 15 to 35 nucleobases in length, and wherein the forward primer comprises at least 70%, at least 80%, at least 90%, at least 95%, or at least 100% sequence identity with a sequence selected from the group consisting of SEQ ID NOS: 2-30, 60-71 and 84-97, and the reverse primer comprises at least 70% sequence identity with a sequence selected from the group consisting of SEQ ID NOS: 31-59, 72-83, and 98-111.

50. The system of claim 47, wherein said primer pair is selected from the group of primer pair sequences consisting of: SEQ ID NOS: 2:31, 3:32, 4:33, 5:34, 6:35, 7:36, 8:37, 9:38, 10:39, 11:40, 12:41, 13:42, 4:43, 15:44, 16:45, 17:46, 18:47, 19:48, 20:49, 21:50, 22:51, 23:52, 24:53, 25:54, 26:55, 27:56, 28:57, 29:58, 30:59, 60:72, 61:73, 62:74, 63:75, 64:76, 65:77, 66:78, 67:79, 68:80, 69:81, 70:82, 71:83, 84:98, 85:99, 86:100, 87:101, 88:102, 89:103, 90:104, 91:105, 92:106, 93:107, 94:108, 95:109, 96:110, and 97:111.

51. The system of claim 47, wherein said controller is configured to determine base compositions of said amplicons from said molecular masses of said amplicons, which base compositions correspond to said one or more picornavirus bioagent identities.

52. The system of claim 47, wherein said controller comprises or is operably connected to a database of known molecular masses and/or known base compositions of amplicons of known picornavirus bioagents produced with the primer pair.
